(19) United States
(12) Patent Application Publication
(10) Pub. No.: US 2009/0170846 A1
(43) Pub. Date: Jul. 2, 2009

(54) INHIBITORS OF THE HIV INTEGRASE ENZYME

(75) Inventors: Klaus Ruprecht Dress, San Diego, CA (US); Michael Bruno Plewe, San Diego, CA (US); Ted William Johnson, San Diego, CA (US); Steven Paul Tanis, Carlsbad, CA (US); Khanh Tuan Tran, San Diego, CA (US)

Correspondence Address: PFIZER INC;Mary J Hosley, 150 EAST 42ND STREET, 5TH FLOOR - STOP 49S, NEW YORK, NY, 10017-5612, US

(73) Assignee: Pfizer Inc

(21) Appl. No.: 12/088,583

(22) PCT Filed: Sep. 22, 2006
(86) PCT No.: PCT/IB2006/002731

§ 371 Date: Aug. 28, 2008

Related U.S. Application Data
(60) Provisional application No. 60/723,237, filed on Oct. 3, 2005.
(60) Provisional application No. 60/724,485, filed on Oct. 6, 2005.
(60) Provisional application No. 60/761,464, filed on Jan. 24, 2006.

(51) Int. Cl. A61K 31/5377; C07D 471/04; A61K 31/437; C07D 413/14; A61P 31/18; C12N 7/06

(52) U.S. Cl. 514/234.5; 546/113; 514/300; 544/127; 435/238

(57) ABSTRACT

The present invention is directed to compounds of formula (I), and pharmaceutically acceptable salts and solvates thereof, their synthesis, and their use as modulators or inhibitors of the human immunodeficiency virus “HIV” integrase enzyme. Formula (I).

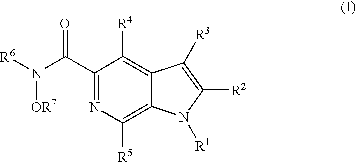

### FIELD

The present invention is directed to compounds, and pharmaceutically acceptable salts and solvates thereof, their synthesis, and their use as modulators or inhibitors of the human immunodeficiency virus (“HIV”) integrase enzyme. The compounds of the present invention are useful for modulating (e.g. inhibiting) an enzyme activity of HIV integrase enzyme and for treating diseases or conditions mediated by HIV, such as for example, acquired immunodeficiency syndrome (“AIDS”), and AIDS related complex (“ARC”).

### BACKGROUND

The retrovirus designated “human immunodeficiency virus” or “HIV” is the etiological agent of a complex disease that progressively destroys the immune system. The disease is known as acquired immune deficiency syndrome or AIDS. AIDS and other HIV-caused diseases are difficult to treat due to the ability of HIV to rapidly replicate, mutate and acquire resistance to drugs. In order to slow the proliferation of the virus after infection, treatment of AIDS and other HIV-caused diseases has focused on inhibiting HIV replication.

Since HIV is a retrovirus, and thus, encodes a positive-sense RNA strand, its mechanism of replication is based on the conversion of viral RNA to viral DNA, and subsequent insertion of the viral DNA into the host cell genome. HIV replication relies on three constitutive HIV encoded enzymes: reverse transcriptase (RT), protease and integrase.

Upon infection with HIV, the retroviral core particles bind to specific cellular receptors and gain entry into the host cell cytoplasm. Once inside the cytoplasm, viral RT catalyzes the reverse transcription of viral ssRNA to form viral RNA-DNA hybrids. The RNA strand from the hybrid is then partially degraded and a second DNA strand is synthesized resulting in viral dsDNA. Integrase, aided by viral and cellular proteins, then transports the viral dsDNA into the host cell nucleus as a component of the pre-integration complex (PIC). In addition, integrase provides the permanent insertion, i.e., integration, of the viral dsDNA to the host cell genome, which, in turn, provides viral access to the host cellular machinery for gene expression. Following integration, transcription and translation produce viral precursor proteins.

A key step in HIV replication, insertion of the viral dsDNA into the host cell genome, is believed to be mediated by integrase in at least three, and possibly, four, steps: (1) assembly of proviral DNA; (2) 3′-end processing causing assembly of the PIC; (3) 3′-end joining or DNA strand transfer, i.e., integration; and (4) gap filling, a repair function. See, e.g., Goldgur, Y. et al., PNAS 96(23): 13040-13043 (November 1999); Sayasith, K. et al., Expert Opin. Ther. Targets 5(4): 443-464 (2001); Young, S. D., Curr. Opin. Drug Disc. & Devel. 4(4): 402-410 (2001); Wai, J. S. et al., J. Med. Chem. 43(26): 4923-4926 (2000); Debyser, Z. et al., Assays for the Evaluation of HIV-1 Integrase Inhibitors, from Methods in Molecular Biology 160: 139-155, Schein, C. H. (ed.), Humana Press Inc., Totowa, N.J. (2001); and Hazuda, D. et al., Drug Design and Disc. 13:17-24 (1997).

Currently, AIDS and other HIV-caused disease are treated with an “HIV cocktail” containing multiple drugs including RT and protease inhibitors. However, numerous side effects and the rapid emergence of drug resistance limit the ability of the RT and protease inhibitors to safely and effectively treat AIDS and other HIV-caused diseases. In view of the shortcomings of RT and protease inhibitors, there is a need for another mechanism through which HIV replication can be inhibited. Integration, and thus integrase, a virally encoded enzyme with no mammalian counterpart, is a logical alternative. See, e.g., Wai, J. S. et al., J. Med. Chem. 43:4923-4926 (2000); Grobler, J. et al., PNAS 99: 6661-6666 (2002); Pais, G. C. G. et al., J. Med. Chem. 45: 3184-3194 (2002); Young, S. D., Curr. Opin. Drug Disc. & Devel. 4(4): 402-410 (2001); Godwin, C. G. et al., J. Med. Chem. 45: 3184-3194 (2002); Young, S. D. et al., “L-870, 810: Discovery of a Potent HIV Integrase Inhibitor with Potential Clinical Utility,” Poster presented at the XIV International AIDS Conference, Barcelona (Jul. 7-12, 2002); and WO 02/070491.

It has been suggested that for an integrase inhibitor to function, it should inhibit the strand transfer integrase function. See, e.g., Young, S. D., Curr. Opin. Drug Disc. & Devel. 4(4): 402-410 (2001). Thus, there is a need for HIV inhibitors, specifically, integrase inhibitors, and, more specifically, strand transfer inhibitors, to treat AIDS and other HIV-caused diseases. The inventive agents disclosed herein are novel, potent and selective HIV-integrase inhibitors, and, more specifically, strand transfer inhibitors, with high antiviral activity.

### SUMMARY

The present invention provides compounds of formula (I),

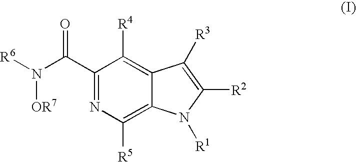

wherein:

R¹ is hydrogen, C₁-C₈ alkyl, C₂-C₈ alkenyl, or C₁-C₈ heteroalkyl, wherein said C₁-C₈ alkyl, C₂-C₈ alkenyl, or C₁-C₈ heteroalkyl groups may be optionally substituted with at least one substituent independently selected from:
halo, —OR^{12a}, —N(R^{12a}R^{12b}), —C(O)N(R^{12a}R^{12b}), —NR^{12a}C(O)N(R^{12a}R^{12b}), —NR^{12a}C(O)R^{12a}, —NR^{12a}C(NR^{12a})N(R^{12a}R^{12b}), —SR^{12a}, S(O)R^{12a}, S(O)₂R^{12a}, —S(O)₂N(R^{12a}R^{12b})₂, C₁-C₈ alkyl, C₆-C₁₄ aryl, C₃-C₈ cycloalkyl, and C₂-C₉ heteroaryl, wherein said C₁-C₈ alkyl, C₆-C₁₄ aryl, C₃-C₈ cycloalkyl, and C₂-C₉ heteroaryl groups are optionally substituted with at least one substituent independently selected from halo, —C(R^{12a}R^{12b}R^{12c}), —OH, and C₁-C₈ alkoxy;

R² is hydrogen;

R³ is —NR⁸C(O)R⁹, —NR⁸S(O)R⁹, —NR⁸S(O)₂R⁹, —C(O)NR⁸R⁹, —S(O)NR⁸R⁹, or —S(O)₂NR⁸R⁹;

R⁴ is hydrogen, halo, C₁-C₈ alkyl, —OR^{12a}, —NR^{12a}R^{12b}, C₁-C₈ heteroalkyl C₂-C₈ alkenyl, or C₂-C₈ alkynyl, wherein said C₁-C₈ alkyl, C₁-C₈ heteroalkyl, C₂-C₈ alkenyl or C₂-C₈ alkynyl groups are optionally substituted with at least one R¹³;

R⁵ is hydrogen;

R⁶ is hydrogen, C₁-C₈ alkyl, C₁-C₈ heteroalkyl, or C₂-C₈ alkenyl, wherein said C₂-C₈ alkenyl is optionally substituted with at least one —OR^{12a} group;

R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with at least one C₃-C₈ cycloalkyl or C₆-C₁₄ aryl group;

each R⁸ and R⁹, which may be the same or different, is independently selected from hydrogen, C₁-C₈ alkyl, C₃-C₈ cycloalkyl, C₆-C₁₂ aryl, C₂-C₈ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group; or

R⁸ and R⁹, together with the nitrogen atom to which they are attached, form a C₂-C₉ heterocyclyl or a C₂-C₉ heteroaryl group, each of which is optionally substituted with at least one R¹⁰ group;

each R¹⁰ is independently selected from halo, C₁-C₈ alkyl, C₃-C₈ cycloalkyl, C₆-C₁₂ aryl, C₂-C₉ heterocyclyl, C₂-C₉ heteroaryl, —(CR^{12a}R^{12b})ₜOR⁷, —C(O)R^{12a}, —S(O)₂R⁷, (CR^{12a}R^{12b})_{z}C(O)NR^{12a}R^{12b}, —NR^{12a}R^{12b}, and —CF₃, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted with at least one R¹⁴ group;

each R^{12a}, R^{12b}, and R^{12c}, which may be the same or different, is independently selected from hydrogen and C₁-C₈ alkyl;

each R¹³, which may be the same or different, is independently selected from —OR^{12a}, halo, C₆-C₁₄ aryl, C₂-C₉ heteroaryl, C₁-C₈ heteroalkyl, C₃-C₈ cycloalkyl, C₂-C₉ heterocyclyl, and —C(R^{12a}R^{12b}R^{12c});

each R¹⁴, which may be the same or different, is independently selected from halogen, C₁-C₈ alkyl, C₃-C₈ cycloalkyl, —CF₃, and —OR^{12a};

each z, which may be the same or different, is independently selected and is 0, 1, or 2; and

each t, which may be the same or different, is independently selected and is 0, 1, 2, or 3; or

a pharmaceutically acceptable salt or solvate thereof.

In another embodiment are provided compounds of formula (I), wherein R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy, or a pharmaceutically acceptable salt or solvate thereof. In yet another embodiment are provided compounds of formula (I), wherein R¹ is C₁-C₈ alkyl, wherein said C₁-C₈ alkyl is substituted with C₆-C₁₄ aryl, wherein said C₆-C₁₄ aryl is optionally substituted with at least one halo, or a pharmaceutically acceptable salt or solvate thereof. In another embodiment are provided compounds of formula (I), wherein R¹ is —(CH₂)(C₆-C₁₄ aryl), wherein said C₆-C₁₄ aryl is optionally substituted with at least one halo, or a pharmaceutically acceptable salt or solvate thereof. In another embodiment are provided compounds of formula (I), wherein R¹ is —(CH₂)(C₆-C₁₄ aryl), wherein said C₆-C₁₄ aryl is optionally substituted with at least one fluorine, or a pharmaceutically acceptable salt or solvate thereof. In yet another embodiment are provided compounds of formula (I), wherein R¹ is 4-fluorobenzyl, or a pharmaceutically acceptable salt or solvate thereof.

In another embodiment are provided compounds of formula (I), wherein R³ is —NR⁸C(O)R⁹, —NR⁸S(O)R⁹, or —NR⁸S(O)₂R⁹, or a pharmaceutically acceptable salt or solvate thereof. In another embodiment are compounds of formula (I), wherein R³ is —NR⁸C(O)R⁹, or a pharmaceutically acceptable salt or solvate thereof. In another embodiment are compounds of formula (I), wherein R³ is —NR⁸S(O)R⁹, or a pharmaceutically acceptable salt or solvate thereof. In another embodiment are compounds of formula (I), wherein R³ is —NR⁸S(O)₂R⁹, or a pharmaceutically acceptable salt or solvate thereof. In yet another embodiment are compounds of formula (I), wherein R³ is —C(O)NR⁸R⁹, —S(O)NR⁸R⁹ or —S(O)₂NR⁸R⁹, or a pharmaceutically acceptable salt or solvate thereof. In yet another embodiment are compounds of formula (I), wherein R³ is —C(O)NR⁸R⁹, or a pharmaceutically acceptable salt or solvate thereof. In still another embodiment are compounds of formula (I), wherein R³ is —S(O)NR⁸R⁹ or —S(O)₂NR⁸R⁹, or a pharmaceutically acceptable salt or solvate thereof. In still another embodiment are compounds of formula (I), wherein R³ is —S(O)NR⁸R⁹, or a pharmaceutically acceptable salt or solvate thereof. In still another embodiment are compounds of formula (I), wherein R³ is —S(O)₂NR⁸R⁹, or a pharmaceutically acceptable salt or solvate thereof.

In another embodiment are provided compounds of formula (I), wherein R⁴ is hydrogen, or a pharmaceutically acceptable salt or solvate thereof.

In another embodiment are provided compounds of formula (I), wherein R⁶ is hydrogen or C₁-C₈ alkyl, or a pharmaceutically acceptable salt or solvate thereof. In another embodiment are compounds of formula (I), wherein R⁶ is hydrogen or —CH₃, or a pharmaceutically acceptable salt or solvate thereof. In another embodiment are compounds of formula (I), wherein R⁶ is hydrogen, or a pharmaceutically acceptable salt or solvate thereof. In another embodiment are compounds of formula (I), wherein R⁶ is C₁-C₈ alkyl, or a pharmaceutically acceptable salt or solvate thereof. In another embodiment are compounds of formula (I), wherein R⁶ is —CH₃, or a pharmaceutically acceptable salt or solvate thereof.

In still another embodiment are provided compounds of formula (I), wherein R⁷ is hydrogen or C₁-C₈ alkyl, or a pharmaceutically acceptable salt or solvate thereof. In another embodiment are provided compounds wherein R⁷ is hydrogen or —CH₃, or a pharmaceutically acceptable salt or solvate thereof. In another embodiment are compounds of formula (I), wherein R⁷ is hydrogen, or a pharmaceutically acceptable salt or solvate thereof. In another embodiment are compounds of formula (I), wherein R⁷ is C₁-C₈ alkyl, or a pharmaceutically acceptable salt or solvate thereof. In another embodiment are compounds of formula (I), wherein R⁷ is —CH₃, or a pharmaceutically acceptable salt or solvate thereof.

In another embodiment are provided compounds of formula (I), wherein each R⁸ and R⁹, which may be the same or different, is independently selected from hydrogen, C₁-C₈ alkyl, C₃-C₈ cycloalkyl, C₆-C₁₂ aryl, C₂-C₉ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group, or a pharmaceutically acceptable salt or solvate thereof.

In still another embodiment are provided compounds of formula (I), wherein R⁸ and R⁹, together with the nitrogen atom to which they are attached, form a C₂-C₉ heterocyclyl or a C₂-C₉ heteroaryl group, each of which is optionally substituted with at least one R¹⁰ group, or a pharmaceutically acceptable salt or solvate thereof. In yet another embodiment are compounds of formula (I), wherein R⁸ and R⁹, together with the nitrogen atom to which they are attached, form a C₂-C₉ heterocyclyl group, which is optionally substituted with at least one R¹⁰ group, or a pharmaceutically acceptable salt or solvate thereof. In another embodiment are compounds of formula (I), wherein R⁸ and R⁹, together with the nitrogen atom to which they are attached, form a C₂-C₉ heteroaryl group, which is optionally substituted with at least one R¹⁰ group, or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy;

R³ is —NR⁸C(O)R⁹, —NR⁸S(O)R⁹, —NR⁸S(O)₂R⁹, —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, which may be the same or different, is independently selected from hydrogen, C₁-C₈ alkyl, C₃-C₈ cycloalkyl, C₆-C₁₂ aryl, C₂-C₉ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group; and

wherein R¹⁰ is as defined above;

or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl, wherein said C₆-C₁₄ aryl is optionally substituted with at least one halo;

R³ is —NR⁸C(O)R⁹, —NR⁸S(O)R⁹, —NR⁸S(O)₂R⁹, —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, which may be the same or different, is independently selected from hydrogen, C₁-C₈ alkyl, C₃-C₈ cycloalkyl, C₆-C₁₂ aryl, C₂-C₉ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group; and

wherein R¹⁰ is as defined above;

or a pharmaceutically acceptable salt or solvate thereof.

In another embodiment are provided compounds of formula (I), wherein:

R¹ is —(CH₂)(C₆-C₁₄ aryl), wherein said C₆-C₁₄ aryl is optionally substituted with at least one halo;

R³ is —NR⁸C(O)R⁹, —NR⁸S(O)R⁹, —NR⁸S(O)₂R⁹, —C(O)NR⁸R⁹, —S(O)NR⁸R⁹, or —S(O)₂NR⁸R⁹;

R⁴ is hydrogen;

R⁶ is hydrogen or C₁-C₈ alkyl n;

R⁷ is hydrogen or C₁-C₈ alkyl;

R⁸ and R⁹, which may be the same or different, is independently selected from hydrogen, C₁-C₈ alkyl, C₃-C₈ cycloalkyl, C₆-C₁₂ aryl, C₂-C₉ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group; and

wherein R¹⁰ is as defined above;

or a pharmaceutically acceptable salt or solvate thereof.

In another embodiment are provided compounds of formula (I), wherein:

R¹ is —(CH₂)(C₆-C₁₄ aryl), wherein said C₆-C₁₄ aryl is optionally substituted with at least one fluorine;

R³ is —NR⁸C(O)R⁹, —NR⁸S(O)R⁹, —NR⁸S(O)₂R⁹, —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, which may be the same or different, is independently selected from hydrogen, C₁-C₈ alkyl, C₃-C₈ cycloalkyl, C₆-C₁₂ aryl, C₂-C₈ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group; and

wherein R¹⁰ is as defined above;

or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy;

R³ is —NR⁸C(O)R⁹, —NR⁸S(O)R⁹, or —NR⁸S(O)₂R⁹;

R⁴ is hydrogen;

R⁶ is hydrogen or C₁-C₈ alkyl;

R⁷ is hydrogen or C₁-C₈ alkyl; and

R⁸ and R⁹, which may be the same or different, is independently selected from hydrogen, C₁-C₈ alkyl, C₃-C₈ cycloalkyl, C₆-C₁₂ aryl, C₂-C₉ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group;

wherein R¹⁰ is as defined above;

or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy;

R³ is —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, which may be the same or different, is independently selected from hydrogen, C₁-C₈ alkyl, C₃-C₈ cycloalkyl, C₆-C₁₂ aryl, C₂-C₉ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group; and

wherein R¹⁰ is as defined above;

or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy;

R³ is —NR⁸C(O)R⁹, —NR⁸S(O)R⁹, —NR⁸S(O)₂R⁹, —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy;

R³ is —NR⁸C(O)R⁹, —NR⁸S(O)R⁹, or —NR⁸S(O)₂R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₈ heteroaryl group, each of which is optionally substituted with at least one R¹⁰ group; and

wherein R¹ is as defined above;

or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl, wherein said C₆-C₁₄ aryl is optionally substituted with at least one halo;

R³ is —NR⁸C(O)R⁹, —NR⁸S(O)R⁹, or —NR⁸S(O)₂R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is —(CH₂)(C₆-C₁₄ aryl), wherein said C₆-C₁₄ aryl is optionally substituted with at least one halo;

R³ is —NR⁸C(O)R⁹, —NR⁸S(O)R⁹, or —NR⁸S(O)₂R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is —(CH₂)(C₆-C₁₄ aryl), wherein said C₆-C₁₄ aryl is optionally substituted with at least one fluorine;

R³ is —NR⁸C(O)R⁹, —NR⁸S(O)R⁹, or —NR⁸S(O)₂R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is —(CH₂)(C₆-C₁₄ aryl), wherein said C₆-C₁₄ aryl is optionally substituted with at least one fluorine;

R³ is —NR⁸C(O)R⁹, —NR⁸S(O)R⁹, or —NR⁸S(O)₂R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is —(CH₂)(C₆-C₁₄ aryl), wherein said C₆-C₁₄ aryl is optionally substituted with at least one fluorine;

R³ is —NR⁸C(O)R⁹, —NR⁸S(O)R⁹, or —NR⁸S(O)₂R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl group, which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is 4-fluorobenzyl;

R³ is —NR⁸C(O)R⁹, —NR⁸S(O)R⁹, or —NR⁸S(O)₂R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heteroaryl group, which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy;

R³ is —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is C₁-C₈ alkyl, wherein said C₁-C₈ alkyl is substituted with C₆-C₁₄ aryl, wherein said C₆-C₁₄ aryl is optionally substituted with at least one halo;

R³ is —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is —(CH₂)(C₆-C₁₄ aryl), wherein said C₆-C₁₄ aryl is optionally substituted with at least one halo;

R³ is —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is —(CH₂)(C₆-C₁₄ aryl), wherein said C₆-C₁₄ aryl is optionally substituted with at least one fluorine;

R³ is —C(O)NR⁸R⁹, —S(O)NR⁸R⁹, or —S(O)₂NR⁸R⁹;

R⁴ is hydrogen or C₁-C₈ alkyl;

R⁶ is hydrogen;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is 4-fluorobenzyl;

R³ is —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl or a C₂-C₉ heteroaryl group, each of which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹⁰ is 4-fluorobenzyl;

R¹⁰ is —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heterocyclyl group, which is optionally substituted with at least one R¹⁰ group; and

wherein R¹⁰ is as defined above;

or a pharmaceutically acceptable salt or solvate thereof.

In another embodiment are provided compounds of formula (I), wherein:

R¹ is 4-fluorobenzyl;

R³ is —C(O)NR⁸R⁹, —S(O)NR⁸R⁹, or —S(O)₂NR⁸R⁹;

R⁴ is hydrogen;

R⁶ is hydrogen or C₁-C₈ alkyl;

R⁷ is hydrogen or C₁-C₈ alkyl;

R⁸ and R⁹, together with the nitrogen atom to which they are attached, form a C₂-C₉ heteroaryl group, which is optionally substituted with at least one R¹⁰ group; and

wherein R¹⁰ is as defined above;

or a pharmaceutically acceptable salt or solvate thereof.

In another embodiment are provided compounds selected from 3-(acetylamino)-1-(4-fluorobenzyl)-N-hydroxy-1H-pyrrolo[2,3-c]pyridine-5-carboxamide; 3-(acetylamino)-1-(4-fluorobenzyl)-N-hydroxy-N-methyl-1H-pyrrolo[2,3-c]pyridine-5-carboxamide; 1-(4fluorobenzyl)-N-hydroxy-3-[(phenylsulfonyl)amino]-1H-pyrrolo[2,3-c]pyridine-5-carboxamide; 1-(4-fluorobenzyl)-N-hydroxy-N-methyl-3-[(phenylsulfonyl)amino]-1H-pyrrolo[2,3-c]pyridine-5-carboxamide; 1-(4-fluorobenzyl)-N-hydroxy-3-[(methylsulfonyl)amino]-1H-pyrrolo[2,3-c]pyridine-5-carboxamide; 1-(4-fluorobenzyl)-N-hydroxy-N-methyl-3-[(methylsulfonyl)amino]-1H-pyrrolo[2,3-c]pyridine-5-carboxamide; 1-(4-fluorobenzyl)-N˜5˜-methoxy-N˜3˜-(2-morpholin-4-ylethyl)-1H-pyrrolo[2,3-c]pyridine-3,5-dicarboxamide; N˜3˜-[(1-ethylpyrrolidin-2-yl)methyl]-1-(4-fluorobenzyl)-N˜5˜-methoxy-1H-pyrrolo[2,3-c]pyridine-3,5-dicarboxamide; 1-(4-fluorobenzyl)-N˜5˜-methoxy-N˜3˜-[3-(2-oxopyrrolidin-1-yl)propyl]-1H-pyrrolo[2,3-c]pyridine-3,5-dicarboxamide; 1-(4-fluorobenzyl)-3-{[(2S)-2-(hydroxymethyl)pyrrolidin-1-yl]carbonyl}-N-methoxy-1H-pyrrolo[2,3-c]pyridine-5-carboxamide; N˜3˜-[(1,5-dimethyl-1H-pyrazol-4-yl)methyl]-1-(4-fluorobenzyl)-N˜5˜-methoxy-1H-pyrrolo[2,3-c]pyridine-3,5-dicarboxamide; 3-{[3-(dimethylamino)pyrrolidin-1-yl]carbonyl}-1-(4-fluorobenzyl)-N-methoxy-1H-pyrrolo[2,3-c]pyridine-5-carboxamide; 1-(4-fluorobenzyl)-3-{[3-(hydroxymethyl)piperidin-1-yl]carbonyl}-N-methoxy-1H-pyrrolo[2,3-c]pyridine-5-carboxamide; 1-(4-fluorobenzyl)-N˜5˜-hydroxy-N˜5˜-methyl-N˜3˜-(tetrahydro-1H-pyrrolizin-7a(5H)-ylmethyl)-1H-pyrrolo[2,3-c]pyridine-3,5-dicarboxamide; N˜3˜-[1-cyclopropyl-3-(cyclopropylamino)-3-oxopropyl]-1-(4-fluorobenzyl)-N˜5˜-methoxy-1H-pyrrolo[2,3-c]pyridine-3,5-dicarboxamide; 1-(4-fluorobenzyl)-N˜5˜-hydroxy-N˜5˜-methyl-N˜3˜-[3-(2-oxopyrrolidin-1-yl)propyl]-1H-pyrrolo[2,3-c]pyridine-3,5-dicarboxamide; N˜3˜-[(1,5-dimethyl-1H-pyrazol-4-yl)methyl]-1-(4-fluorobenzyl)-N˜5˜-hydroxy-N˜5˜-methyl-1H-pyrrolo[2,3-c]pyridine-3,5-dicarboxamide; N˜3˜-[1-cyclopropyl-3-(cyclopropylamino)-3-oxopropyl]-1-(4-fluorobenzyl)-N˜5˜-hydroxy-N˜5˜-methyl-1H-pyrrolo[2,3-c]pyridine-3,5-dicarboxamide; 3-[(benzylsulfonyl)amino]-1-(4-fluorobenzyl)-N-hydroxy-1H-pyrrolo[2,3-c]pyridine-5-carboxamide; 1-(4-fluorobenzyl)-N-hydroxy-3-[(1,2,3,4-tetrahydroisoquinolin-7-ylsulfonyl)amino]-1H-pyrrolo[2,3-c]pyridine-5-carboxamide; 3-{[(5-chloro-2-thienyl)sulfonyl]amino}-1-(4-fluorobenzyl)-N-hydroxy-1H-pyrrolo[2,3-c]pyridine-5-carboxamide; 1-(4-fluorobenzyl)-N-hydroxy-3-{[(2S)-2-(hydroxymethyl)pyrrolidin-1-yl]carbonyl}-N-methyl-1H-pyrrolo[2,3-c]pyridine-5-carboxamide; 1-(4-fluorobenzyl)-N-hydroxy-3-{[(2S)-2-(hydroxymethyl)pyrrolidin-1-yl]carbonyl}-1H-pyrrolo[2,3-c]pyridine-5-carboxamide; N˜3˜-[1-cyclopropyl-3-(cyclopropylamino)-3-oxopropyl]-1-(4-fluorobenzyl)-N˜5˜-hydroxy-1H-pyrrolo[2,3-c]pyridine-3,5-dicarboxamide; 1-(4-fluorobenzyl)-N˜5˜-hydroxy-N˜3˜-(tetrahydro-1H-pyrrolizin-7a(5H)-ylmethyl)-1H-pyrrolo[2,3-c]pyridine-3,5-dicarboxamide; 3-{[3-(dimethylamino)pyrrolidin-1-yl]carbonyl}-1-(4-fluorobenzyl)-N-hydroxy-1H-pyrrolo[2,3-c]pyridine-5-carboxamide; 1-(4-fluorobenzyl)-N˜5˜-hydroxy-N˜3˜-(2-morpholin-4-ylethyl)-1H-pyrrolo[2,3-c]pyridine-3,5-dicarboxamide; N˜3˜-[(1,5-dimethyl-1H-pyrazol-4-yl)methyl]-1-(4-fluorobenzyl)-N˜5˜-hydroxy-1H-pyrrolo[2,3-c]pyridine-3,5-dicarboxamide; 1-(4-fluorobenzyl)-N˜5˜-hydroxy-N˜3˜-[3-(2-oxopyrrolidin-1-yl)propyl]-1H-pyrrolo[2,3-c]pyridine-3,5-dicarboxamide; 1-(4-fluorobenzyl)-N-hydroxy-3-{[3-(hydroxymethyl)piperidin-1-yl]carbonyl}-1H-pyrrolo[2,3-c]pyridine-5-carboxamide; N˜3˜-[(1-ethylpyrrolidin-2-yl)methyl]-1-(4-fluorobenzyl)-N˜5˜-hydroxy-1H-pyrrolo[2,3-c]pyridine-3,5-dicarboxamide; 1-(4-fluorobenzyl)-N˜5˜-hydroxy-N˜5˜-methyl-N˜3˜-(2-morpholin-4-ylethyl)-1H-pyrrolo[2,3-c]pyridine-3,5-dicarboxamide; 1-(4-fluorobenzyl)-N-hydroxy-3-{[3-(hydroxymethyl)piperidin-1-yl]carbonyl}-N-methyl-1H-pyrrolo[2,3-c]pyridine-5-carboxamide; 3-[(dimethylamino)sulfonyl]-1-(4-fluorobenzyl)-N-hydroxy-1H-pyrrolo[2,3-c]pyridine-5-carboxamide; 3-[(dimethylamino)sulfonyl]-1-(4-fluorobenzyl)-N-hydroxy-N-methyl-1H-pyrrolo[2,3-c]pyridine-5-carboxamide; 3-{[(2S)-2-(aminocarbonyl)pyrrolidin-1-yl]sulfonyl}-1-(2,4-difluorobenzyl)-N-hydroxy-1H-pyrrolo[2,3-c]pyridine-5-carboxamide; 1-(2,4-difluorobenzyl)-N-hydroxy-3-{[(2-morpholin-4-ylethyl)amino]sulfonyl}-1H-pyrrolo[2,3-c]pyridine-5-carboxamide; 1-(2,4-difluorobenzyl)-N-hydroxy-3-[(3-oxopiperazin-1-yl)sulfonyl]-1H-pyrrolo[2,3-c]pyridine-5-carboxamide; 1-(2,4-difluorobenzyl)-N-methoxy-3-[(3-oxopiperazin-1-yl)sulfonyl]-1H-pyrrolo[2,3-c]pyridine-5-carboxamide; 1-(2,4-difluorobenzyl)-N-methoxy-3-{[(2-morpholin-4-ylethyl)amino]sulfonyl}-1H-pyrrolo[2,3-c]pyridine-5-carboxamide; 1-(2,4-difluorobenzyl)-N-hydroxy-N-methyl-3-[(3-oxopiperazin-1-yl)sulfonyl]-1H-pyrrolo[2,3-c]pyridine-5-carboxamide; 3-{[(2S)-2-(aminocarbonyl)pyrrolidin-1-yl]sulfonyl}-1-(2,4-difluorobenzyl)-N-hydroxy-N-methyl-1H-pyrrolo[2,3-c]pyridine-5-carboxamide; 1-(4-fluorobenzyl)-N5-hydroxy-N5-methyl-N3-[(2S)-tetrahydrofuran-2-ylmethyl]-1H-pyrrolo[2,3-c]pyridine-3,5-dicarboxamide; 1-(4-fluorobenzyl)-N5-hydroxy-N3-isopropyl-N5-methyl-1H-pyrrolo[2,3-c]pyridine-3,5-dicarboxamide; N3-(2,2-difluoroethyl)-1-(4-fluorobenzyl)-N5-hydroxy-N5-methyl-1H-pyrrolo[2,3-c]pyridine-3,5-dicarboxamide; 1-(4-fluorobenzyl)-3-{[(2R)-2-(hydroxymethyl)pyrrolidin-1-yl]carbonyl}-N-methoxy-1H-pyrrolo[2,3-c]pyridine-5-carboxamide; 1-(4-fluorobenzyl)-N3-isopropyl-N5-methoxy-1H-pyrrolo[2,3-c]pyridine-3,5-dicarboxamide; N3-(2,2-difluoroethyl)-1-(4-fluorobenzyl)-N5-methoxy-1H-pyrrolo[2,3-c]pyridine-3,5-dicarboxamide; 3-[(diethylamino)sulfonyl]-1-(4-fluorobenzyl)-N-methoxy-1H-pyrrolo[2,3-c]pyridine-5-carboxamide; 1-(4-fluorobenzyl)-N-hydroxy-3-{[(3-hydroxypropyl)amino]sulfonyl}-N-methyl-1H-pyrrolo[2,3-c]pyridine-5-carboxamide; 1-(4-fluorobenzyl)-3-{[(3-hydroxypropyl)amino]sulfonyl}-N-methoxy-1H-pyrrolo[2,3-c]pyridine-5-carboxamide; 1-(4-fluorobenzyl)-N-methoxy-3-{[(2-methoxypyridin-3-yl)amino]sulfonyl}-1H-pyrrolo[2,3-c]pyridine-5-carboxamide; 1-(4-fluorobenzyl)-N-hydroxy-3-{[(2-methoxypyridin-3-yl)amino]sulfonyl}-N-methyl-1H-pyrrolo[2,3-c]pyridine-5-carboxamide; 3-{[(1,4-dioxan-2-ylmethyl)(methyl)amino]sulfonyl}-1-(4-fluorobenzyl)-N-methoxy-1H-pyrrolo[2,3-c]pyridine-5carboxamide; 1-(4-fluorobenzyl)-N-methoxy-3-(morpholin-4-ylsulfonyl)-1H-pyrrolo[2,3-c]pyridine-5-carboxamide; 3-{[(1,4-dioxan-2-ylmethyl)(methyl)amino]sulfonyl}-1-(4-fluorobenzyl)-N-hydroxy-N-methyl-1H-pyrrolo[2,3-c]pyridine-5-carboxamide; and 1-(4-fluorobenzyl)-N-hydroxy-N-methyl-3-(morpholin-4-ylsulfonyl)-1H-pyrrolo[2,3-c]pyridine-5-carboxamide; or

a pharmaceutically acceptable salt or solvate thereof.

In a further aspect are provided pharmaceutical compositions, comprising a therapeutically effective amount of at least one of any of the compounds herein, or a pharmaceutically acceptable salt or solvate thereof, and a pharmaceutically acceptable carrier or diluent.

Further provided are methods of inhibiting HIV replication in a mammal, comprising administering to said mammal an HIV-inhibiting amount of at least one of any of the compounds herein, or a pharmaceutically acceptable salt or solvate thereof.

Also afforded herein are methods of inhibiting HIV replication in a cell, comprising contacting said cell with an HIV-inhibiting amount of at least one of any of the compounds herein, or a pharmaceutically acceptable salt or solvate thereof.

Still further are provided methods of inhibiting HIV integrase enzyme activity, comprising contacting said integrase enzyme with a HIV integrase-inhibiting amount of at least one of any of the compounds herein, or a pharmaceutically acceptable salt or solvate thereof.

In yet another aspect of the present invention are afforded methods of treating acquired immune deficiency syndrome in a mammal, comprising administering to said mammal a therapeutically effective amount of at least one of any of the compounds herein, or a pharmaceutically acceptable salt or solvate thereof.

Further provided are methods of inhibiting HIV replication in a mammal, wherein said HIV is resistant to at least one HIV protease inhibitor, said method comprising administering to said mammal a therapeutically effective amount of at least one of any of the compounds herein, or a pharmaceutically acceptable salt or solvate thereof.

Also afforded herein are methods of inhibiting HIV replication in a mammal, wherein said HIV is resistant to at least one HIV reverse transcriptase inhibitor, said methods comprising administering to said mammal a therapeutically effective amount of at least one of any of the compounds herein, or a pharmaceutically acceptable salt or solvate thereof.

Further provided herein are methods of inhibiting HIV replication in mammal, comprising administering to said mammal a therapeutically effective amount of at least one of any of the compounds herein, or a pharmaceutically acceptable salt or solvate thereof, and at least one other anti-HIV agent.

Also provided herein are methods of reducing HIV viral load in a mammal infected with HIV, comprising administering to said mammal a therapeutically effective amount of at least one of any of the compounds herein, or a pharmaceutically acceptable salt or solvate thereof.

Further provided are uses of compounds herein, or a pharmaceutically acceptable salt or solvate thereof, in the manufacture of a medicament for the treatment of acquired immune deficiency syndrome (AIDS) or AIDS-related complex in an HIV-infected mammal, such as a human.

As used herein, the terms “comprising” and “including” are used in their open, non-limiting sense.

As used herein, the term “HIV” means Human Immunodeficiency Virus. The term “HIV integrase,” as used herein, means the Human Immunodeficiency Virus integrase enzyme.

The term “C₁-C₈ alkyl,” as used herein, means saturated monovalent hydrocarbon radicals having straight or branched moieties and containing from 1 to 8 carbon atoms. Examples of such groups include, but are not limited to, methyl, ethyl, propyl, iso-propyl, n-butyl, iso-butyl, and tert-butyl.

The term “C₁-C₈ heteroalkyl” refers to a straight- or branched-chain alkyl group having a total of from 2 to 12 atoms in the chain, including from 1 to 8 carbon atoms, and one or more atoms of which is a heteroatom selected from S, O, and N, with the proviso that said chain may not contain two adjacent O atoms or two adjacent S atoms. The S atoms in said chains may be optionally oxidized with one or two oxygen atoms, to afford sulfoxides and sulfones, respectively. Furthermore, the C₁-C₈ heteroalkyl groups in the compounds of the present invention can contain an oxo group at any carbon or heteroatom that will result in a stable compound. Exemplary C₁-C₈ heteroalkyl groups include, but are not limited to, alcohols, alkyl ethers, primary, secondary, and tertiary alkyl amines, amides, ketones, esters, alkyl sulfides, and alkyl sulfones.

The term “C₂-C₈ alkenyl”, as used herein, means an alkyl moiety comprising 2 to 8 carbons having at least one carbon-carbon double bond. The carbon-carbon double bond in such a group may be anywhere along the 2 to 8 carbon chain that will result in a stable compound. Such groups include both the E and Z isomers of said alkenyl moiety. Examples of such groups include, but are not limited to, ethenyl, propenyl, butenyl, allyl, and pentenyl. The term “allyl,” as used herein, means a —CH₂CH═CH₂ group.

As used herein, the term “C₂-C₈ alkynyl” means an alkyl moiety comprising from 2 to 8 carbon atoms and having at least one carbon-carbon triple bond. The carbon-carbon triple bond in such a group may be anywhere along the 2 to 8 carbon chain that will result in a stable compound. Examples of such groups include, but are not limited to, ethyne, propyne, 1-butyne, 2-butyne, 1-pentyne, 2-pentyne, 1-hexyne, 2-hexyne, and 3-hexyne.

The term “C₃-C₈ cycloalkyl group” means a saturated, monocyclic, fused, or spiro, polycyclic ring structure having a total of from 3 to 8 carbon ring atoms. Examples of such groups include, but are not limited to, cyclopropyl, cyclobutyl, cyclopentyl, cyclopentenyl, cyclohexyl, cycloheptyl, and adamantyl.

The term “C₆-C₁₄ aryl”, as used herein, means a group derived from an aromatic hydrocarbon containing from 6 to 14 carbon atoms. Examples of such groups include, but are not limited to, phenyl or naphthyl. The terms “Ph” and “phenyl,” as used herein, mean a —C₆H₅ group. The term “benzyl,” as used herein, means a —CH₂C₆H₅ group.

The term “C₂-C₉ heteroaryl,” as used herein, means an aromatic heterocyclic group having a total of from 5 to 10 atoms in its ring, and containing from 2 to 9 carbon atoms and from one to four heteroatoms each independently selected from O, S and N, and with the proviso that the ring of said group does not contain two adjacent O atoms or two adjacent S atoms. The heterocyclic groups include benzo-fused ring systems. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The C₂-C₉ heteroaryl groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “C₂-C₉ heterocyclyl,” as used herein, means a non-aromatic, monocyclic, bicyclic, tricyclic, or tetracyclic group having a total of from 4 to 10 atoms in its ring system, and containing from 2 to 9 carbon atoms and from one to four heteroatoms each independently selected from O, S and N, and with the proviso that the ring of said group does not contain two adjacent O atoms or two adjacent S atoms. Furthermore, such C₂-C₉ heterocyclyl groups may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a C₂-C₉ heterocyclyl group contains a sulfur atom, said sulfur atom may be oxidized with one or two oxygen atoms to afford either a sulfoxide or sulfone. An example of a 4 membered heterocyclic group is azetidinyl (derived from azetidine). An example of a 5 membered heterocyclic group is thiazolyl and an example of a 10 membered heterocyclic group is quinolinyl. Further examples of such C₂-C₉ heterocyclyl groups include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl.

The term “C₁-C₈ alkoxy”, as used herein, means an O-alkyl group wherein said alkyl group contains from 1 to 8 carbon atoms and is straight, branched, or cyclic. Examples of such groups include, but are not limited to, methoxy, ethoxy, n-propyloxy, iso-propyloxy, n-butoxy, iso-butoxy, tert-butoxy, cyclopentyloxy, and cyclohexyloxy.

The terms “halogen” and “halo,” as used herein, mean fluorine, chlorine, bromine or iodine.

The term “substituted,” means that the specified group or moiety bears one or more substituents. The term “unsubstituted,” means that the specified group bears no substituents. The term “optionally substituted” means that the specified group is unsubstituted or substituted by one or more substituents. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one to which the remainder of the compound of the present invention is bonded, minus an additional substituent, to leave 4). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

The term “solvate,” as used herein, means a pharmaceutically acceptable solvate form of a compound of the present invention that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof. It is specifically contemplated that in the present invention one solvent molecule can be associated with one molecule of the compounds of the present invention, such as a hydrate. Furthermore, it is specifically contemplated that in the present invention, more than one solvent molecule may be associated with one molecule of the compounds of the present invention, such as a dihydrate. Additionally, it is specifically contemplated that in the present invention less than one solvent molecule may be associated with one molecule of the compounds of the present invention, such as a hemihydrate. Furthermore, solvates of the present invention are contemplated as solvates of compounds of the present invention that retain the biological effectiveness of the non-hydrate form of the compounds.

The term “pharmaceutically acceptable salt,” as used herein, means a salt of a compound of the present invention that retains the biological effectiveness of the free acids and bases of the specified derivative and that is not biologically or otherwise undesirable.

The term “pharmaceutically acceptable formulation,” as used herein, means a combination of a compound of the invention, or a pharmaceutically acceptable salt or solvate thereof, and a carrier, diluent, and/or excipients that are compatible with a compound of the present invention, and is not deleterious to the recipient thereof. Pharmaceutical formulations can be prepared by procedures known to those of ordinary skill in the art. For example, the compounds of the present invention can be formulated with common excipients, diluents, or carriers, and formed into tablets, capsules, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as povidone, sodium starch glycolate, sodium carboxymethylcellulose, agar agar, calcium carbonate, and sodium bicarbonate; agents for retarding dissolution such as paraffin; resorption accelerators such as quaternary ammonium compounds; surface/active agents such as cetyl alcohol, glycerol monostearate; adsorptive carriers such as kaolin and bentonite; and lubricants such as talc, calcium and magnesium stearate and solid polyethylene glycols. Final pharmaceutical forms may be pills, tablets, powders, lozenges, saches, cachets, or sterile packaged powders, and the like, depending on the type of excipient used. Additionally, it is specifically contemplated that pharmaceutically acceptable formulations of the present invention can contain more than one active ingredient. For example, such formulations may contain more than one compound according to the present invention. Alternatively, such formulations may contain one or more compounds of the present invention and one or more additional anti-HIV agents.

The term “inhibiting HIV replication” means inhibiting human immunodeficiency virus (HIV) replication in a cell. Such a cell may be present in vitro, or it may be present in vivo, such as in a mammal, such as a human. Such inhibition may be accomplished by administering a compound of the present invention, or a pharmaceutically acceptable salt or solvate thereof, to the cell, such as in a mammal, in an HIV-inhibiting amount. The quantification of inhibition of HIV replication in a cell, such as in a mammal, can be measured using methods known to those of ordinary skill in the art. For example, an amount of a compound of the invention may be administered to a mammal, either alone or as part of a pharmaceutically acceptable formulation. Blood samples may then be withdrawn from the mammal and the amount of HIV virus in the sample may be quantified using methods known to those of ordinary skill in the art. A reduction in the amount of HIV virus in the sample compared to the amount found in the blood before administration of a compound of the invention would represent inhibition of the replication of HIV virus in the mammal. The administration of a compound of the invention to the cell, such as in a mammal, may be in the form of single dose or a series of doses. In the case of more than one dose, the doses may be administered in one day or they may be administered over more than one day.

An “HIV-inhibiting agent” means a compound of the present invention or a pharmaceutically acceptable salt or solvate thereof.

The term “anti-HIV agent,” as used herein, means a compound or combination of compounds capable of inhibiting the replication of HIV in a cell, such as a cell in a mammal. Such compounds may inhibit the replication of HIV through any mechanism known to those of ordinary skill in the art.

The terms “human immunodeficiency virus-inhibiting amount” and “HIV-inhibiting amount,” as used herein, refer to the amount of a compound of the present invention, or a pharmaceutically acceptable salt of solvate thereof, required to inhibit replication of the human immunodeficiency virus (HIV) in vivo, such as in a mammal, or in vitro. The amount of such compounds required to cause such inhibition can be determined without undue experimentation using methods described herein and those known to those of ordinary skill in the art.

The term “inhibiting HIV integrase enzyme activity,” as used herein, means decreasing the activity or functioning of the HIV integrase enzyme either in vitro or in vivo, such as in a mammal, such as a human, by contacting the enzyme with a compound of the present invention.

The term, “HIV integrase enzyme-inhibiting amount,” as used herein, refers to the amount of a compound of the present invention, or a pharmaceutically acceptable salt or solvate thereof, required to decrease the activity of the HIV integrase enzyme either in vivo, such as in a mammal, or in vitro. Such inhibition may take place by the compound of the present invention binding directly to the HIV integrase enzyme. In addition, the activity of the HIV integrase enzyme may be decreased in the presence of a compound of the present invention when such direct binding between the enzyme and the compound does not take place. Furthermore, such inhibition may be competitive, non-competitive, or uncompetitive. Such inhibition may be determined using in vitro or in vivo systems, or a combination of both, using methods known to those of ordinary skill in the art.

The term “therapeutically effective amount,” as used herein, means an amount of a compound of the present invention, or a pharmaceutically acceptable salt or solvate thereof, that, when administered to a mammal in need of such treatment, is sufficient to effect treatment, as defined herein. Thus, a therapeutically effective amount of a compound of the present invention, or a pharmaceutically acceptable salt or solvate thereof, is a quantity sufficient to modulate or inhibit the activity of the HIV integrase enzyme such that a disease condition that is mediated by activity of the HIV integrase enzyme is reduced or alleviated.

The terms “treat”, “treating”, and “treatment” refer to any treatment of an HIV integrase mediated disease or condition in a mammal, particularly a human, and include: (i) preventing the disease or condition from occurring in a subject which may be predisposed to the condition, such that the treatment constitutes prophylactic treatment for the pathologic condition; (ii) modulating or inhibiting the disease or condition, i.e., arresting its development; (iii) relieving the disease or condition, i.e., causing regression of the disease or condition; or (iv) relieving and/or alleviating the disease or condition or the symptoms resulting from the disease or condition, e.g., relieving an inflammatory response without addressing the underlying disease or condition.

The terms “resistant,” “resistance,” and “resistant HIV,” as used herein, refer to HIV virus demonstrating a reduction in sensitivity to a particular drug. A mammal infected with HIV that is resistant to a particular anti-HIV agent or combination of agents usually manifests an increase in HIV viral load despite continued administration of the agent or agents. Resistance may be either genotypic, meaning that a mutation in the HIV genetic make-up has occurred, or phenotypic, meaning that resistance is discovered by successfully growing laboratory cultures of HIV virus in the presence of an anti-HIV agent or a combination of such agents.

The terms “protease inhibitor” and “HIV protease inhibitor,” as used herein, refer to compounds or combinations of compounds that interfere with the proper functioning of the HIV protease enzyme that is responsible for cleaving long strands of viral protein into the separate proteins making up the viral core.

The terms “reverse transcriptase inhibitor” and “HIV reverse transcriptase inhibitor,” as used herein, refer to compounds or combinations of compounds that interfere with the proper functioning of the HIV reverse transcriptase enzyme that is responsible for converting single-stranded HIV viral RNA into HIV viral DNA.

The terms “fusion inhibitor” and “HIV fusion inhibitor,” as used herein, refer to compounds or combinations of compounds that bind to the gp41 envelope protein on the surface of CD4 cells and thereby block the structural changes necessary for the virus to fuse with the cell.

The terms “integrase inhibitor” and “HIV integrase inhibitor,” as used herein, refer to a compound or combination of compounds that interfere with the proper functioning of the HIV integrase enzyme that is responsible for inserting the genes of HIV into the DNA of a host cell.

The term “CCR5 antagonist,” as used herein, refer to compounds or combinations of compounds that block the infection of certain cell types by HIV through the perturbation of CCR5 co-receptor activity.

The terms “viral load” and “HIV viral load,” as used herein, mean the amount of HIV in the circulating blood of a mammal, such as a human. The amount of HIV virus in the blood of mammal can be determined by measuring the quantity of HIV RNA in the blood using methods known to those of ordinary skill in the art.

The term, “compound of the present invention” refers to any of the above-mentioned compounds, as well as those in the Examples that follow, and include those generically described or those described as species. The term also refers to pharmaceutically acceptable salts or solvates of these compounds.

### DETAILED DESCRIPTION

The compounds of the present invention are useful for modulating or inhibiting HIV integrase enzyme. More particularly, the compounds of the present invention are useful as modulators or inhibitors of HIV integrase activity, and thus are useful for the prevention and/or treatment of HIV mediated diseases or conditions (e.g., AIDS, and ARC), alone or in combination with other known antiviral agents.

In accordance with a convention used in the art, the symbol

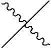

is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g.,

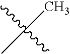

represents a methyl group,

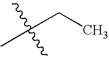

represents an ethyl group

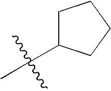

represents a cyclopentyl group, etc.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. The terms “racemic” or “racemic mixture,” as used herein, refer to a 1:1 mixture of enantiomers of a particular compound. The term “diastereomers”, on the other hand, refers to the relationship between a pair of stereoisomers that comprise two or more asymmetric centers and are not mirror images of one another.

The compounds of the present invention may have asymmetric carbon atoms. The carbon-carbon bonds of the compounds of the present invention may be depicted herein using a solid line [symbol] a solid wedge [symbol] or a dotted wedge [symbol] The use of a solid line to depict bonds from asymmetric carbon atoms is meant to indicate that all possible stereoisomers at that carbon atom are included. The use of either a solid or dotted wedge to depict bonds from asymmetric carbon atoms is meant to indicate that only the stereoisomer shown is meant to be included. It is possible that compounds of the invention may contain more than one asymmetric carbon atom. In those compounds, the use of a solid line to depict bonds from asymmetric carbon atoms is meant to indicate that all possible stereoisomers are meant to be included. The use of a solid line to depict bonds from one or more asymmetric carbon atoms in a compound of the invention and the use of a solid or dotted wedge to depict bonds from other asymmetric carbon atoms in the same compound is meant to indicate that a mixture of diastereomers is present.

If a derivative used in the method of the invention is a base, a desired salt may be prepared by any suitable method known to the art, including treatment of the free base with an inorganic acid, such as hydrochloric acid; hydrobromic acid; sulfuric acid; nitric acid; phosphoric acid; and the like, or with an organic acid, such as acetic acid; maleic acid; succinic acid; mandelic acid; fumaric acid; malonic acid; pyruvic acid; oxalic acid; glycolic acid; salicylic acid; pyranosidyl acid, such as glucuronic acid or galacturonic acid; alpha-hydroxy acid, such as citric acid or tartaric acid; amino acid, such as aspartic acid or glutamic acid; aromatic acid, such as benzoic acid or cinnamic acid; sulfonic acid, such as p-toluenesulfonic acid or ethanesulfonic acid; and the like.

If a derivative used in the method of the invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

A “solvate” is intended to mean a pharmaceutically acceptable solvate form of a specified compound that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof.

A “pharmaceutically acceptable salt” is intended to mean a salt that retains the biological effectiveness of the free acids and bases of the specified derivative, containing pharmacologically acceptable anions, and is not biologically or otherwise undesirable. Examples of pharmaceutically acceptable salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phospate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds, agents and salts may exist in different crystal or polymorphic forms, all of which are intended to be within the scope of the present invention and specified formulas.

The compounds of the present invention may be formulated into pharmaceutical compositions as described below in any pharmaceutical form recognizable to the skilled artisan as being suitable. Pharmaceutical compositions of the invention comprise a therapeutically effective amount of at least one compound of the present invention and an inert, pharmaceutically acceptable carrier or diluent.

To treat or prevent diseases or conditions mediated by HIV, a pharmaceutical composition of the invention is administered in a suitable formulation prepared by combining a therapeutically effective amount (i.e., an HIV Integrase modulating, regulating, or inhibiting amount effective to achieve therapeutic efficacy) of at least one compound of the present invention (as an active ingredient) with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol®, Gelucire® or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g., parenteral or oral administration.

To obtain a stable water-soluble dose form, a pharmaceutically acceptable salt of a compound of the present invention may be dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present invention may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use.

In addition to the formulations described above, the compounds of the present invention may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD: 5W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol® Transcutol® and the like may be used. Further, the pharmaceutical composition may be incorporated into a skin patch for delivery of the drug directly onto the skin.

It will be appreciated that the actual dosages of the agents of this invention will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations of the present invention may contain a compound of the present invention, or a pharmaceutically acceptable salt or solvae thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg.

Additionally, the pharmaceutically acceptable formulations of the present invention may contain a compound of the present invention, or a pharmaceutically acceptable salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds of the present invention, or a pharmaceutically acceptable salt or solvate thereof, may be administered to a mammal suffering from infection with HIV, such as a human, either alone or as part of a pharmaceutically acceptable formulation, once a day, twice a day, or three times a day.

Those of ordinary skill in the art will understand that with respect to the compounds of the present invention, the particular pharmaceutical formulation, the dosage, and the number of doses given per day to a mammal requiring such treatment, are all choices within the knowledge of one of ordinary skill in the art and can be determined without undue experimentation. For example, see “Guidelines for the Use of Antiretroviral Agents in HIV-1 Infected Adults and Adolescents,” United States Department of Health and Human Services, available at http://www.aidsinfo.nih.gov/guidelines/ as of Sep. 27, 2005.

The compounds of the present invention may be administered in combination with an additional agent or agents for the treatment of a mammal, such as a human, that is suffering from an infection with the HIV virus, AIDS, AIDS-related complex (ARC), or any other disease or condition which is related to infection with the HIV virus. The agents that may be used in combination with the compounds of the present invention include, but are not limited to, those useful as HIV protease inhibitors, HIV reverse transcriptase inhibitors, non-nucleoside HIV reverse transcriptase inhibitors, inhibitors of HIV integrase, CCR5 inhibitors, HIV fusion inhibitors, compounds useful as immunomodulators, compounds that inhibit the HIV virus by an unknown mechanism, compounds useful for the treatment of herpes viruses, compounds useful as anti-infectives, and others as described below.

Compounds useful as HIV protease inhibitors that may be used in combination with the compounds of the present invention include, but are not limited to, 141 W94 (amprenavir), CGP-73547, CGP-61755, DMP-450, nelfinavir, ritonavir, saquinavir (invirase), lopinavir, TMC-126, atazanavir, palinavir, GS-3333, KN I-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium (Lexiva), benzenesulfonamide derivatives disclosed in WO 03053435, R-944, Ro-03-34649, VX-385, GS-224338, OPT-TL3, PL-100, SM-309515, AG-148, DG-35-VIII, DMP-850, GW-5950X, KNI-1039, L-756423, LB-71262, LP-130, RS-344, SE-063, UIC-94-003, Vb-19038, A-77003, BMS-182193, BMS-186318, SM-309515, JE-2147, GS-9005.

Compounds useful as inhibitors of the HIV reverse transcriptase enzyme that may be used in combination with the compounds of the present invention include, but are not limited to, abacavir, FTC, GS-840, lamivudine, adefovir dipivoxil, beta-fluoro-ddA, zalcitabine, didanosine, stavudine, zidovudine, tenofovir, amdoxovir, SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine, alovudine, .

Compounds useful as non-nucleoside inhibitors of the HIV reverse transcriptase enzyme include, but are not limited to, efavirenz, HBY-097, nevirapine, TMC-120 (dapivirine), TMC-125, etravirine, delavirdine, DPC-083, DPC-961, TMC-120, capravirine, GW-678248, GW-695634, calanolide, and tricyclic pyrimidinone derivatives as disclosed in WO 03062238.

Compounds useful as CCR5 inhibitors that may be used in combination with the compounds of the present invention include, but are not limited to, TAK-779, SC-351125, SCH-D, UK-427857, PRO-140, and GW-873140 (Ono-4128, AK-602).

Compounds useful as inhibitors of HIV integrase enzyme that may be used in combination with the compounds of the present invention include, but are not limited to, GW-810781, L-000810810 (Merck), 1,5-naphthyridine-3-carboxamide derivatives disclosed in WO 03062204, compounds disclosed in WO 03047564, compounds disclosed in WO 03049690, and 5-hydroxypyrimidine-4-carboxamide derivatives disclosed in WO 03035076.

Fusion inhibitors for the treatment of HIV that may be used in combination with the compounds of the present invention include, but are not limited to enfuvirtide (T-20), T-1249, AMD-3100, and fused tricyclic compounds disclosed in JP 2003171381.

Other compounds that are useful inhibitors of HIV that may be used in combination with the compounds of the present invention include, but are not limited to, Soluble CD4, TNX-355, PRO-542, BMS-806, tenofovir disoproxil fumarate, and compounds disclosed in JP 2003119137.

Compounds useful in the treatment or management of infection from viruses other than HIV that may be used in combination with the compounds of the present invention include, but are not limited to, acyclovir, fomivirsen, penciclovir, HPMPC, oxetanocin G, AL-721, cidofovir, cytomegalovirus immune globin, cytovene, fomivganciclovir, famciclovir, foscarnet sodium, Isis 2922, KNI-272, valacyclovir, virazole ribavirin, valganciclovir, ME-609, PCL-016

Compounds that act as immunomodulators and may be used in combination with the compounds of the present invention include, but are not limited to, AD-439, AD-519, Alpha Interferon, AS-101, bropirimine, acemannan, CL246,738, EL10, FP-21399, gamma interferon, granulocyte macrophage colony stimulating factor, IL-2, immune globulin intravenous, IMREG-1, IMREG-2, imuthiol diethyl dithio carbamate, alpha-2 interferon, methionine-enkephalin, MTP-PE, granulocyte colony stimulating sactor, remune, rCD4, recombinant soluble human CD4, interferon alfa-2, SK&F106528, soluble T4 yhymopentin, tumor necrosis factor (TNF), tucaresol, recombinant human interferon beta, and interferon alfa n-3.

Anti-infectives that may be used in combination with the compounds of the present invention include, but are not limited to, atovaquone, azithromycin, clarithromycin, trimethoprim, trovafloxacin, pyrimethamine, daunorubicin, clindamycin with primaquine, fluconazole, pastill, ornidyl, eflornithine pentamidine, rifabutin, spiramycin, intraconazole-R51211, trimetrexate, daunorubicin, recombinant human erythropoietin, recombinant human growth hormone, megestrol acetate, testerone, and total enteral nutrition.

Antifungals that may be used in combination with the compounds of the present invention include, but are not limited to, anidulafungin, C31G, caspofungin, DB-289, fluconzaole, itraconazole, ketoconazole, micafungin, posaconazole, and voriconazole.

Other compounds that may be used in combination with the compounds of the present invention include, but are not limited to, acmannan, ansamycin, LM 427, AR177, BMS-232623, BMS-234475, CI-1012, curdlan sulfate, dextran sulfate, STOCRINE EL10, hypericin, lobucavir, novapren, peptide T octabpeptide sequence, trisodium phosphonoformate, probucol, and RBC-CD4.

In addition, the compounds of the present invention may be used in combination with anti-proliferative agents for the treatment of conditions such as Kaposi's sarcoma. Such agents include, but are not limited to, inhibitors of metallo-matrix proteases, A-007, bevacizumab, BMS-275291, halofuginone, interleukin-12, rituximab, paclitaxel, porfimer sodium, rebimastat, and COL-3.

The particular choice of an additional agent or agents will depend on a number of factors that include, but are not limited to, the condition of the mammal being treated, the particular condition or conditions being treated, the identity of the compound or compounds of the present invention and the additional agent or agents, and the identity of any additional compounds that are being used to treat the mammal. The particular choice of the compound or compounds of the invention and the additional agent or agents is within the knowledge of one of ordinary skill in the art.

The compounds of the present invention may be administered in combination with any of the above additional agents for the treatment of a mammal, such as a human, that is suffering from an infection with the HIV virus, AIDS, AIDS-related complex (ARC), or any other disease or condition which is related to infection with the HIV virus. Such a combination may be administered to a mammal such that a compound or compounds of the present invention are present in the same formulation as the additional agents described above. Alternatively, such a combination may be administered to a mammal suffering from infection with the HIV virus such that the compound or compounds of the present invention are present in a formulation that is separate from the formulation in which the additional agent is found. If the compound or compounds of the present invention are administered separately from the additional agent, such administration may take place concomitantly or sequentially with an appropriate period of time in between. The choice of whether to include the compound or compounds of the present invention in the same formulation as the additional agent or agents is within the knowledge of one of ordinary skill in the art.

Additionally, the compounds of the present invention may be administered to a mammal, such as a human, in combination with an additional agent that has the effect of increasing the exposure of the mammal to a compound of the invention. The term “exposure,” as used herein, refers to the concentration of a compound of the invention in the plasma of a mammal as measured over a period of time. The exposure of a mammal to a particular compound can be measured by administering a compound of the invention to a mammal in an appropriate form, withdrawing plasma samples at predetermined times, and measuring the amount of a compound of the invention in the plasma using an appropriate analytical technique, such as liquid chromatography or liquid chromatography/mass spectroscopy. The amount of a compound of the invention present in the plasma at a certain time is determined and the concentration and time data from all the samples are plotted to afford a curve. The area under this curve is calculated and affords the exposure of the mammal to the compound. The terms “exposure,” “area under the curve,” and “area under the concentration/time curve” are intended to have the same meaning and may be used interchangeably throughout.

Among the agents that may be used to increase the exposure of a mammal to a compound of the present invention are those that can as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir.

Such a combination may be administered to a mammal such that a compound or compounds of the present invention are present in the same formulation as the additional agents described above. Alternatively, such a combination may be administered such that the compound or compounds of the present invention are present in a formulation that is separate from the formulation in which the additional agent is found. If the compound or compounds of the present invention are administered separately from the additional agent, such administration may take place concomitantly or sequentially with an appropriate period of time in between. The choice of whether to include the compound or compounds of the present invention in the same formulation as the additional agent or agents is within the knowledge of one of ordinary skill in the art.

Several different assay formats are available to measure integrase-mediated integration of viral DNA into target (or host) DNA and thus, identify compounds that modulate

(e.g., inhibit) integrase activity. In general, for example, ligand-binding assays may be used to determine interaction with an enzyme of interest. When binding is of interest, a labeled enzyme may be used, wherein the label is a fluoresce, radioisotope, or the like, which registers a quantifiable change upon binding to the enzyme. Alternatively, the skilled artisan may employ an antibody for binding to the enzyme, wherein the antibody is labeled allowing for amplification of the signal. Thus, binding may be determined through direct measurement of ligand binding to an enzyme. In addition, binding may be determined by competitive displacement of a ligand bound to an enzyme, wherein the ligand is labeled with a detectable label. When inhibitory activity is of interest, an intact organism or cell may be studied, and the change in an organismic or cellular function in response to the binding of the inhibitory compound may be measured. Alternatively, cellular response can be determined microscopically by monitoring viral induced cytopathic effects, syncytium-formation (HIV-1 syncytium-formation assays), for example. Thus, there are various in vitro and in vivo assays useful for measuring HIV integrase inhibitory activity. See, e.g., Lewin, S. R. et al., Journal of Virology 73(7): 6099-6103 (July 1999); Hansen, M. S. et al., Nature Biotechnology 17(6): 578-582 (June 1999); and Butler, S. L. et al., Nature Medicine 7(5): 631-634 (May 2001).

Exemplary specific assay formats used to measure integrase-mediated integration include, but are not limited to, ELISA, DELFIA® (PerkinElmer Life Sciences Inc. (Boston, Mass.)) and ORIGEN® (IGEN International, Inc. (Gaithersburg, Md.)) technologies. In addition, gel-based integration (detecting integration by measuring product formation with SDS-PAGE) and scintillation proximity assay (SPA) disintegration assays that use a single unit of double stranded-DNA (ds-DNA) may be used to monitor integrase activity.

In one embodiment of the invention, the preferred assay is an integrase strand-transfer SPA (stINTSPA) which uses SPA to specifically measure the strand-transfer mechanism of integrase in a homogenous assay scalable for miniaturization to allow high-throughput screening. The assay focuses on strand transfer and not on DNA binding and/or 3′ processing. This sensitive and reproducible assay is capable of distinguishing non-specific interactions from true enzymatic function by forming 3′ processed viral DNA/integrase complexes before the addition of target DNA. Such a formation creates a bias toward compound modulators (e.g., inhibitors) of strand-transfer and not toward compounds that inhibit integrase 3′ processing or prevent the association of integrase with viral DNA. This bias renders the assay more specific than known assays. In addition, the homogenous nature of the assay reduces the number of steps required to run the assay since the wash steps of a heterogenous assay are not required.

The integrase strand-transfer SPA format consists of 2 DNA components that model viral DNA and target DNA. The model viral DNA (also known as donor DNA) is biotinylated ds-DNA preprocessed at the 3′ end to provide a CA nucleotide base overhang at the 5′ end of the duplex. The target DNA (also known as host DNA) is a random nucleotide sequence of ds-DNA generally containing [³H]-thymidine nucleotides on both strands, preferably, at the 3′ ends, to enable detection of the integrase strand-transfer reaction that occurs on both strands of target ds-DNA.

Integrase (created recombinantly or synthetically and preferably, purified) is pre-complexed to the viral DNA bound to a surface, such as for example, streptavidin-coated SPA beads. Generally, the integrase is pre-complexed in a batch process by combining and incubating diluted viral DNA with integrase and then removing unbound integrase. The preferred molar ratio of viral DNA:integrase is about 1:about 5. The integrase/viral DNA incubation is optional, however, the incubation does provide for an increased specificity index with an integrase/viral DNA incubation time of about 15 to about 30 minutes at room temperature or at about 37° C. The preferred incubation is at about 37° C. for about 15 minutes.

The reaction is initiated by adding target DNA, in the absence or presence of a potential integrase modulator compound, to the integrase/viral DNA beads (for example) and allowed to run for about 20 to about 50 minutes (depending on the type of assay container employed), at about room temperature or about 37° C., preferably, at about 37° C. The assay is terminated by adding stop buffer to the integrase reaction mixture. Components of the stop buffer, added sequentially or at one time, function to terminate enzymatic activity, dissociate integrase/DNA complexes, separate non-integrated DNA strands (denaturation agent), and, optionally, float the SPA beads to the surface of the reaction mixture to be closer in range to the detectors of, for example, a plate-based scintillation counter, to measure the level of integrated viral DNA which is quantified as light emitted (radiolabeled signal) from the SPA beads. The inclusion of an additional component in the stop buffer, such as for example CsCl or functionally equivalent compound, is optionally, and preferably, used with a plate-based scintillation counter, for example, with detectors positioned above the assay wells, such as for example a TopCount® counter (PerkinElmer Life Sciences Inc. (Boston, Mass.)). CsCl would not be employed when PMT readings are taken from the bottom of the plate, such as for example when a MicroBeta® counter (PerkinElmer Life Sciences Inc. (Boston, Mass.)) is used.

The specificity of the reaction can be determined from the ratio of the signal generated from the target DNA reaction with the viral DNA/integrase compared to the signal generated from the di-deoxy viral DNA/integrase. High concentrations (e.g., ≧50 nM) of target DNA may increase the d/dd DNA ratio along with an increased concentration of integrase in the integrase/viral DNA sample.

The results can be used to evaluate the integrase modulatory, such as for example inhibitory, activity of test compounds. For example, the skilled artisan may employ a high-throughput screening method to test combinatorial compound libraries or synthetic compounds. The percent inhibition of the compound may be calculated using an equation such as for example (1−((CPM sample−CPM min)/(CPM max−CPM min)))*100. The min value is the assay signal in the presence of a known modulator, such as for example an inhibitor, at a concentration about 100-fold higher than the IC₅₀ for that compound. The min signal approximates the true background for the assay. The max value is the assay signal obtained for the integrase-mediated activity in the absence of compound. In addition, the IC₅₀ values of synthetic and purified combinatorial compounds may be determined whereby compounds are prepared at about 10 or 100-fold higher concentrations than desired for testing in assays, followed by dilution of the compounds to generate an 8-point titration curve with ½-log dilution intervals, for example. The compound sample is then transferred to an assay well, for example. Further dilutions, such as for example, a 10-fold dilution, are optional. The percentage inhibition for an inhibitory compound, for example, may then be determined as above with values applied to a nonlinear regression, sigmoidal dose response equation (variable slope) using GraphPad Prism curve fitting software (GraphPad Software, Inc., San Diego, Calif.) or functionally equivalent software.

The stINTSPA assay conditions are preferably optimized for ratios of integrase, viral DNA and target DNA to generate a large and specific assay signal. A specific assay signal is defined as a signal distinguishing true strand-transfer catalytic events from complex formation of integrase and DNA that does not yield product. In other integrase assays, a large non-specific component (background) often contributes to the total assay signal unless the buffer conditions are rigorously optimized and counter-tested using a modified viral DNA oligonucleotide. The non-specific background is due to formation of integrase/viral DNA/target DNA complexes that are highly stable independent of a productive strand-transfer mechanism.

The preferred stINTSPA distinguishes complex formation from productive strand-transfer reactions by using a modified viral DNA oligonucleotide containing a di-deoxy nucleoside at the 3′ end as a control. This modified control DNA can be incorporated into integrase/viral DNA/target DNA complexes, but cannot serve as a substrate for strand-transfer. Thus, a distinct window between productive and non-productive strand-transfer reactions can be observed. Further, reactions with di-deoxy viral DNA beads give an assay signal closely matched to the true background of the assay using the preferred optimization conditions of the assay. The true background of the assay is defined as a reaction with all assay components (viral DNA and [³H]-target DNA) in the absence of integrase.

Assay buffers used in the integrase assay generally contain at least one reducing agent, such as for example 2-mercaptoethanol or DTT, wherein DTT as a fresh powder is preferred; at least one divalent cation, such as for example Mg⁺⁺, Mn⁺⁺, or Zn⁺⁺, preferably, Mg⁺⁺; at least one emulsifier/dispersing agent, such as for example octoxynol (also known as IGEPAL-CA or NP-40) or CHAPS; NaCl or functionally equivalent compound; DMSO or functionally equivalent compound; and at least one buffer, such as for example MOPS. Key buffer characteristics are the absence of PEG; inclusion of a high concentration of a detergent, such as for example about 1 to about 5 mM CHAPS and/or about 0.02 to about 0.15% IGEPAL-CA or functionally equivalent compound(s) at least capable of reducing non-specific sticking to the SPA beads and assay wells and, possibly, enhancing the specificity index; inclusion of a high concentration of DMSO (about 1 to about 12%); and inclusion of modest levels of NaCl (≦50 mM) and MgCl₂ (about 3 to about 10 mM) or functionally equivalent compounds capable of reducing the dd-DNA background. The assay buffers may optionally contain a preservative, such as for example NaN₃, to reduce fungal and bacterial contaminants during storage.

The stop buffer preferably contains EDTA or functionally equivalent compound capable of terminating enzymatic activity, a denaturation agent comprising, for example, NaOH or guanidine hydrochloride, and, optionally, CsCl or functionally equivalent compound capable of assisting in floating the SPA beads to the top of the assay container for scintillation detection at the top of the reservoir and, possibly, minimizing compound interference. An example of an integrase strand-transfer SPA is set forth in Example 43.

Alternatively, the level of activity of the modulatory compounds may be determined in an antiviral assay, such as for example an assay that quantitatively measures the production of viral antigens (e.g., HIV-1 p24) or the activities of viral enzymes (e.g., HIV-1 reverse transcriptase) as indicators of virus replication, or that measures viral replication by monitoring the expression of an exogenous reporter gene introduced into the viral genome (HIV-1 reporter virus assays) (Chen, B. K. et al., J. Virol. 68(2): 654-660 (1994); Terwilliger, E. F. et al., PNAS 86:3857-3861 (1989)). A preferred method of measuring antiviral activity of a potential modulator compound employs an HIV-1 cell protection assay, wherein virus replication is measured indirectly by monitoring viral induced host-cell cytopathic effects using, for example, dye reduction methods as set forth in Example 44.

In one embodiment, the compounds of the present invention include those having an EC₅₀ value against HIV integrase of at least 10⁻⁵ M (or at least 10 μM) when measured with an HIV cell protection assay. In another embodiment are compounds of the present invention with an EC₅₀ value against HIV integrase of at least 1 μM when measured with an HIV cell protection assay. In yet another embodiment, the compounds of the present invention have an EC₅₀ against HIV integrase of at least 0.1 μM when measured with an HIV cell protection assay.

The inventive agents may be prepared using the reaction routes and synthesis schemes as described below, employing the techniques available in the art using starting materials that are readily available. The preparation of certain embodiments of the present invention is described in detail in the following examples, but those of ordinary skill in the art will recognize that the preparations described may be readily adapted to prepare other embodiments of the present invention. For example, the synthesis of non-exemplified compounds according to the invention may be performed by modifications apparent to those skilled in the art, e.g., by appropriately protecting interfering groups, by changing to other suitable reagents known in the art, or by making routine modifications of reaction conditions. Alternatively, other reactions disclosed herein or known in the art will be recognized as having adaptability for preparing other compounds of the invention.

### General Procedures

The compounds of the present invention can be prepared directly from compound 1-1 (preferably a methyl or ethyl ester) and a substituted or unsubstituted hydroxylamine in the presence of a base, such as, for example, sodium hydroxide or sodium alkoxide in methanol or ethanol (Hauser, C. R., et al., Org. Synth. Coll. Vol. 2, p. 67, John Wiley, New York (1943)). Alternatively, the compound 1-1 can be saponified to the free acid 1-2 using lithium hydroxide or sodium hydroxide in methanol/water mixtures and heating the mixture to 100° C. in a SmithCreator® microwave for 1 to 5 min. Compound 1-2 can be coupled with a substituted or unsubstituted hydroxylamine using a coupling reagent. Typical coupling reagents and conditions can be used, such as, for example, O-(azabenzotriazole-1-yl)-1,1,3,3-tetramethyl uronium hexafluorophosphate (HATU), N-(3-dimethylaminopropyl)-N′-ethylcarbodiimide (EDC) in DMF at ambient temperature, or many others that are familiar to those skilled in the art. Other suitable methods are described, for example, in M. B. Smith, J. March, Advanced Organic Chemistry, 5th edition, John Whiley & Sons, p. 508-511 (2001). The use of the preferred conditions described in this scheme would allow for parallel preparation or combinatorial libraries of such hydroxamates 1-3.

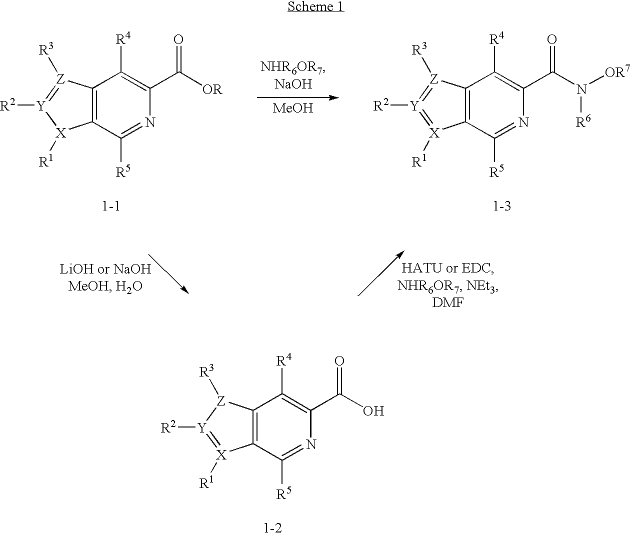

### Preparation of Intermediates and Starting Materials

The precursors of type 1-1 with X═N, Y═C, Z=C (Compound 2-7) can be prepared from an arylsulfonyl or alkylsulfonyl protected pyrrole compound 2-2 formed from pyrrole compound 2-1 and an arylsulfonylchloride or an alkylsulfonylchloride in the presence of a base, such as, for example, triethylamine, using methods described, for example, in T. W. Greene, Protective Groups in Organic Chemistry, 3^{rd} edition, John Wiley & Sons, pp. 615-617 (1999). Reductive amination with a suitable substituted glycine ester compound 2-3 and a reducing agent, such as, for example, NaBH₃CN or NaBH(OAc)₃ (Abdel-Magid, A. F. et al., Tetrahedron Lett., 31, 5595-5598 (1990)) can provide the amine compound 2-4. Additional methods for reductive amination exist and are reviewed in C. F. Lane, Synthesis, p. 135 (1975). Titanium tetrachloride mediated cyclization (Dekhane, M. et al., Tetrahedron, 49, pp. 8139-8146 (1993); and Singh, S. K., Heterocycles, 44, pp. 379-391 (1997)) in a solvent, such as, for example, benzene or toluene, at the boiling temperature of the solvent can provide the arylsulfonyl or alkylsulfonyl protected precursor compound 2-5, which can be converted to the desired unprotected indole compound 2-6 using sodium alkoxide in alcohol (M. Dekhane, P. Potier, R. H. Dodd, Tetrahedron, 49, 8139-8146 (1993)). Alkylation of compound 2-6 with an alkylhalide in a polar solvent such as DMF or DMSO using sodium hydride as base (Eberle, M. K., J. Org. Chem., 41, pp. 633-636 (1976); Sundberg, R. J. et al., J. Org. Chem. 38, pp. 3324-3330 (1973)) can provide the desired precursor compound 2-7.

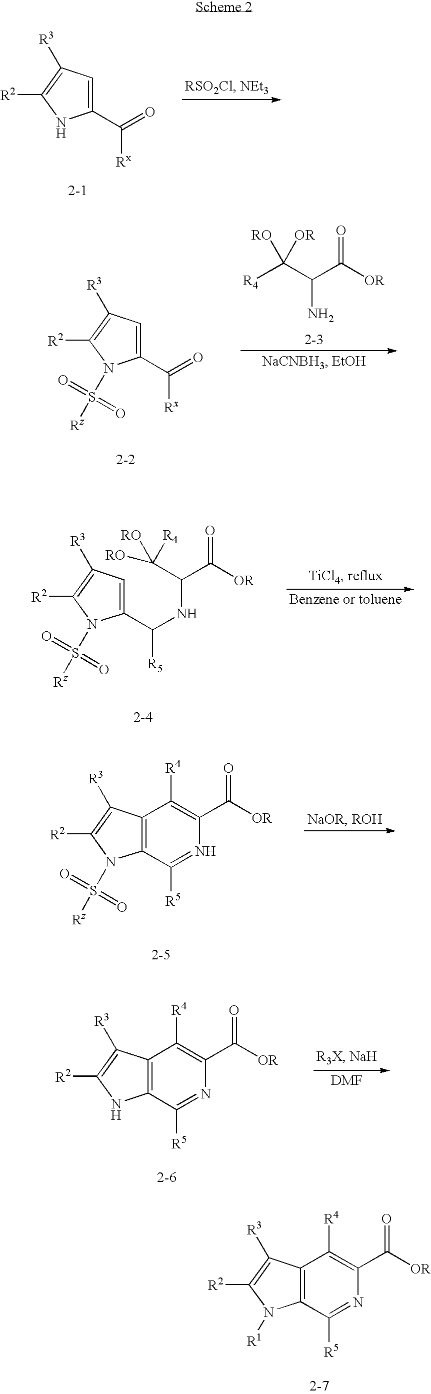

Scheme 3 depicts an alternative method for obtaining intermediate compound 2-5 adapted from the literature (Rousseau, J. F. et al., J. Org. Chem., 63, pp. 2731-2737 (1998) and citations therein) starting from the substituted pyrrole compound 3-1. The pyrrole nitrogen can be protected as a sulfonamide using the same methods described in Scheme 2. Addition of the anion of an N-Cbz glycine ester can provide the intermediate compound 3-4. Removal of the Cbz protecting group can be achieved using palladium catalyzed hydrogenation or other methods, such as those described in T. W. Greene, P. G. M. Wuts, Protective Groups in Organic Chemistry, 3^{rd} edition, John Wiley & Sons, pp. 531-537 (1999). Pictet-Spengler condensation followed by palladium catalyzed dehydrogenation in xylene can afford the intermediate compound 2-5.

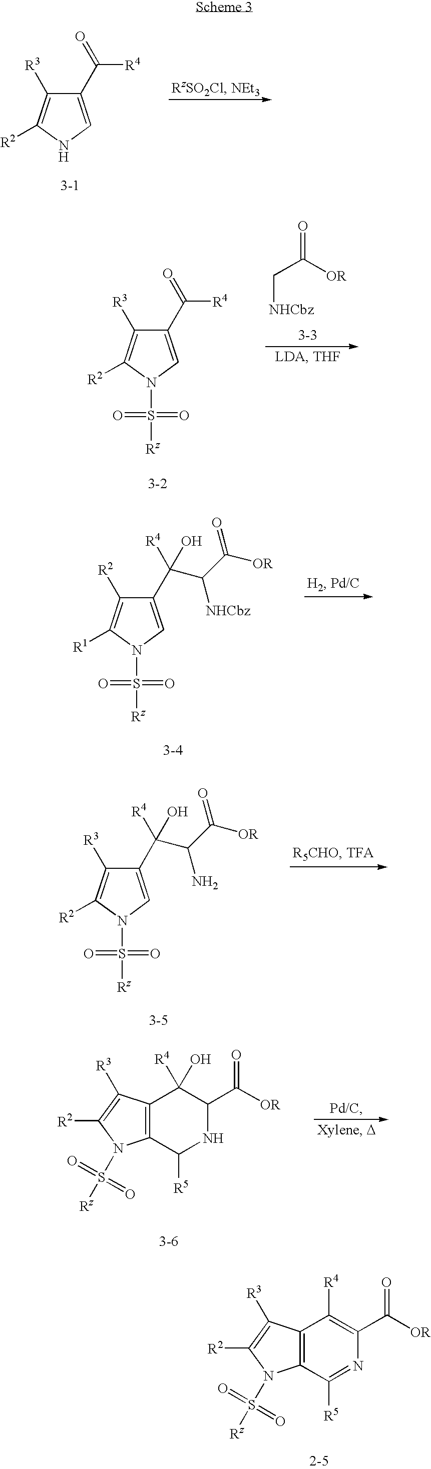

Scheme 4 depicts an alternative method for the formation of the azaindole core 4-9. The hydroxypyridine 4-1 can be converted to the corresponding triflate or bromide 4-2 using POBr₃ or trifluoromethanesulfonic anhydride and a base such as triethylamine. Reaction of 4-2 with zinc cyanide in the presence of a catalyst such as Pd(PPh₃)₄ (D. M. Tschaen et al. Synthetic Comm. 1994, 24, 887-890) can provide nitrile 4-3, which can be converted to ester 4-4 under acidic conditions. Reaction of 4-4 with dimethylformamide dimethyacetal followed by reduction can provide azaindole 4-6 (Prokopov, A. A. et al. Khim. Geterotsikl. Soedin. 1977, 1135, M. Sloan, R. S. Philipps, Bioorg. Med. Chem. Lett., 1992, 2, 1053-1056), which can be alkylated to 4-7 using a alkyl or benzyl halide and a base such as sodium hydride. Formylation of the pyrrole ring system in 4-7 can be accomplished using 1,1-dichloromethylmethyl ether in the presence of aluminum chloride as described by X. Doisy e. al. Bioorg. Med. Chem. 1999, 7, 921-932 to provide compound 4-8, which can react with an amine and a reducing agent such as sodium triacetoxy borohydride to provide 4-9.

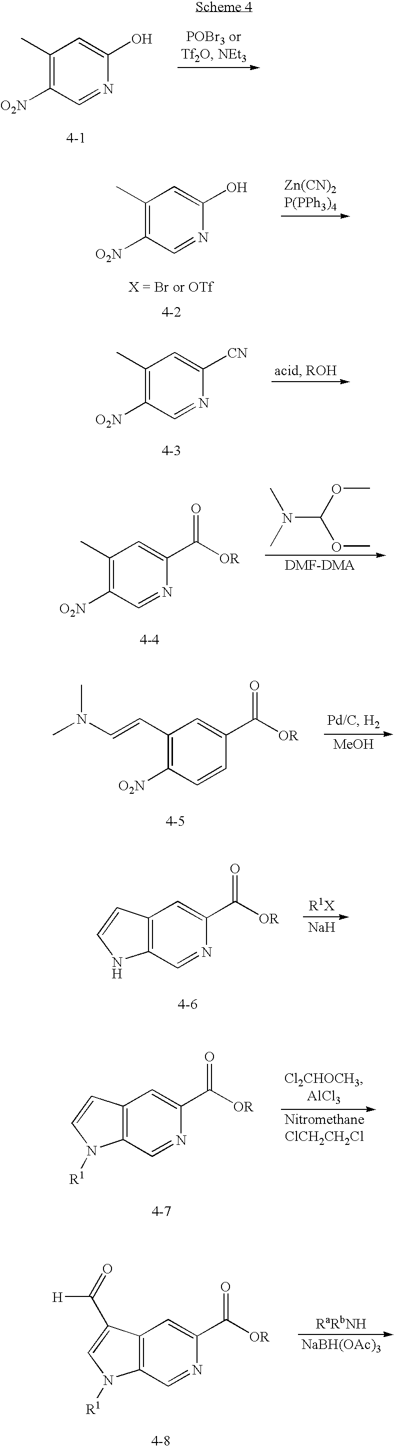

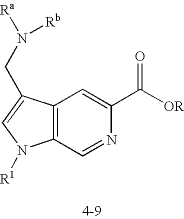

An alternative route that can provide 3-substituted pyrrolo[2,3-c]pyridines 5-6 and 5-7 from the unsubstituted precursor 5-1 is depicted in Scheme 5. Reaction of compound 5-1 with dimethylmethyleneimmonium chloride (A. P. Kozikowski, H. Ishida, Heterocycles 1980, 14, 55-58) can give the dimethylaminomethyl derivative 5-2. Alternatively, this step can be performed using classic Mannich reaction conditions (review: J. H. Brewster, E. L. Eliel, Org. Reactions, 1953, 7, 99). Upon treatment of 5-2 with sodium acetate and acetic anhydride in acetonitrile (J. N. Cocker, O. B. Mathre, W. H. Todd, J. Org. Chem., 1963, 28, 589-590) the corresponding acetate 5-3 can be obtained, which, on hydrolysis with a base such as potassium carbonate in methanol, can provide the precursor 5-5. Alkylation of the alcohol 5-5 can be achieved using an alkylhalide in the presence of a base such as sodium hydride in DMF as solvent to give 5-7. Alternatively, 5-2 can be treated with ethyl chloroformate (Shinohara, H.; Fukuda, T. and Iwao, M., Tetrahedron, 1999, 55, 10989-11000) to form chloride 5-4 which can react with a thiol or alcohol to form 5-6. as described by Naylor, M. A., et al. J. Med. Chem., 1998, 41, 2720-2731.

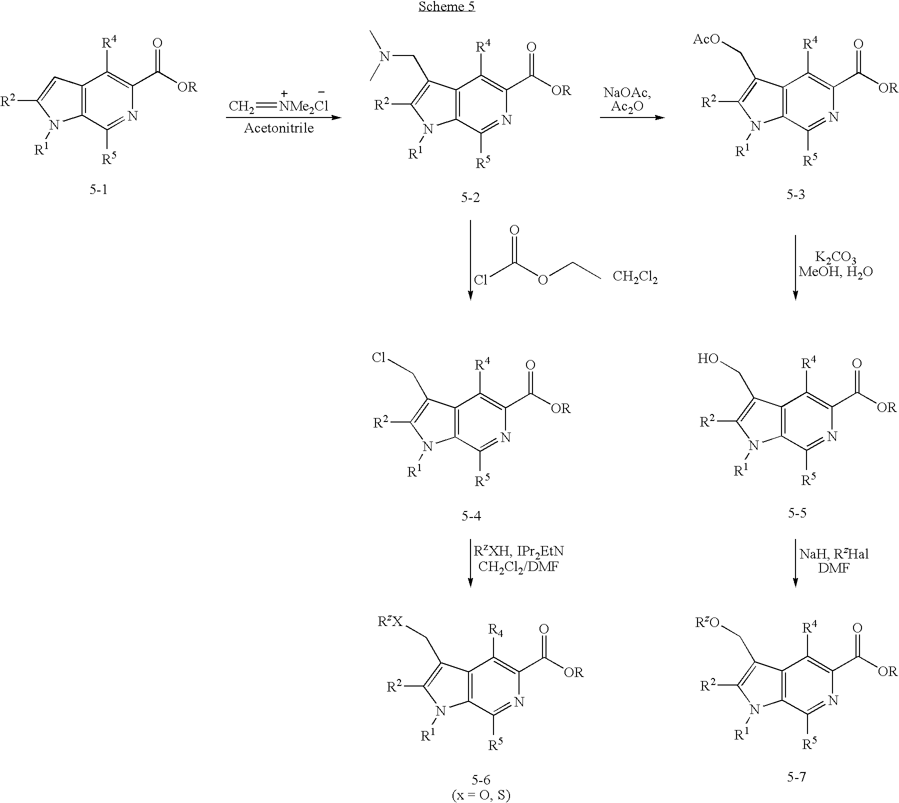

Imidazo[4,5-c]pyridine derivatives of type 1-1 (X═N, Y═C, Z=N) can be obtained according to Scheme 6. The histidine precursor 6-1 is commercially available or can be prepared according published methods (J. L. Kelley, C. A. Miller, E W. McLean, J. Med. Chem. 1977, 20, 721-723, G. Trout, J. Med. Chem. 1972, 15, 1259-1261). Pictet-Spengler reaction of 6-1 (F. Guzman et al., J. Med. Chem. 1984, 27, 564-570, M. Cain, F. Guzman, M. Cook, Heterocycles, 1982, 19, 1003-1007) can give the 1,2,3,4-tetrahydro-imidazo[4,5-c]pyridine-3-carboxylate 6-2, which can be converted to the methyl ester 6-3 via the corresponding acyl chloride or similar methods of ester formation known to those skilled in the art. Dehydrogenation to the unsaturated intermediate 6-4 can be achieved with selenium dioxide (J. G. Lee, K. C. Kim, Tetrahedron Lett, 1992, 33, 6363-6366), or a catalyst such as palladium or platinum in a solvent such as xylene at the boiling temperature of the solvent (D. Soerens, et al., J. Org. Chem., 1979, 44, 535-545). Alkylation of 6-4 with an alkylhalide in the presence of a base such as sodium hydride similar to the methods described in Scheme 2 can provide the desired precursors as a mixture of regioisomers 6-5 and 6-6 that can be separated by column chromatography or other methods known to those skilled in the art.

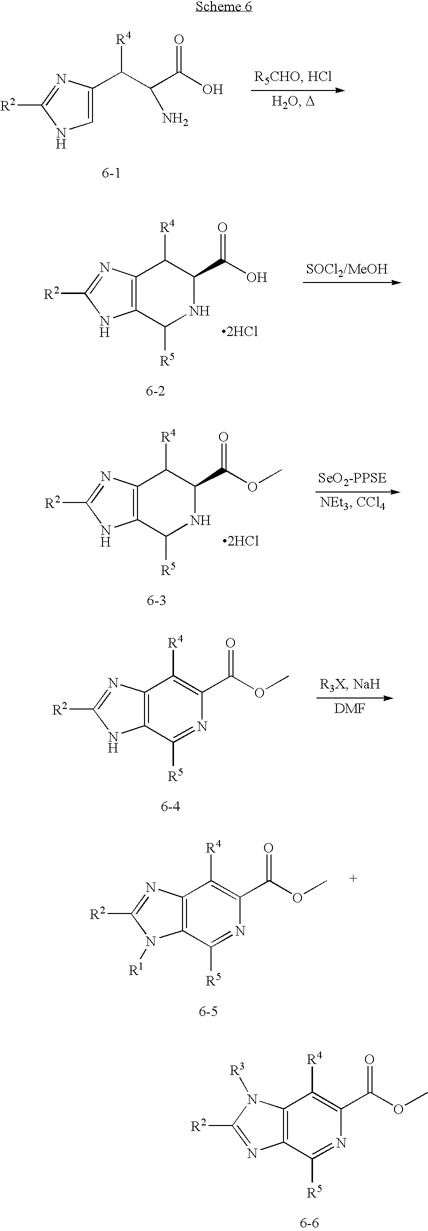

Scheme 7 sets forth a method for producing pyrrolo[3,2-c]pyridine derivatives 1-1 where X═C, Y═C, Z=N, and preferably R=an alkyl group (compound 7-3) via a substituted pyrrole compound of type 7-1 and a 2-azabutadiene compound of type 7-2 (Kantlehner, W., et al., Liebigs Ann. Chem., pp. 344-357 (1980)) under proton catalysis, following the procedures described in Biere, H., et al., Liebigs Ann. Chem., pp. 491-494 (1987). Friedel-Crafts acylation can provide ketone 7-5 which upon reduction with a reducing agent such as borane-t-butyl amine complex in THF can give compound 7-6 and alcohol 7-7.

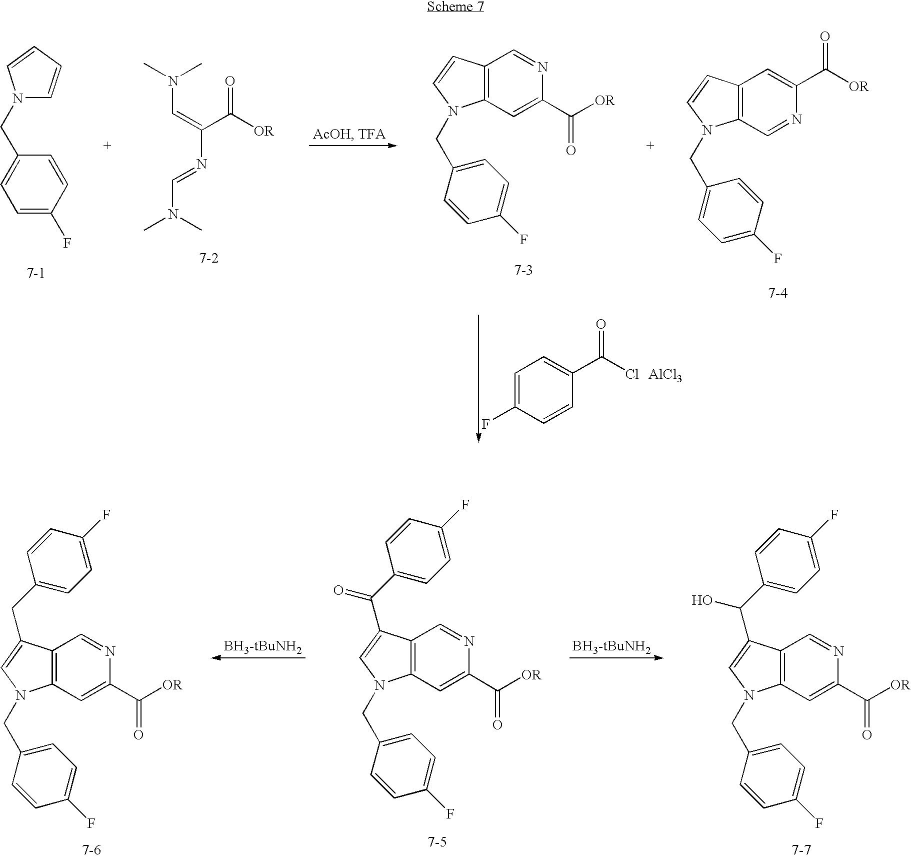

Scheme 8 depicts a general method (T. L. Gilchrist, C. W. Rees, J. A. R. Rodriguez, J. C. S. Chem. Comm. 1979, 627-628, L. Henn, D. M. B. Hickey, C. J. Moody, C. W. Rees, J. Chem. Soc. Perkin Trans. 1 1984, 2189-2196, A. Shafiee, H. Ghazar, J. Heterocyclic Chem. 1986, 23, 1171-1173) for the formation of compounds of general structure 1-1. Reaction of a substituted heteroaromatic aldehyde or ketone 8-1 with ethyl or methyl azidoacetate 8-2 in the presence of a base such as sodium hydride can provide azidocinnamate 8-3, which on thermolysis in boiling toluene or xylenes, can provide the desired product 8-4.

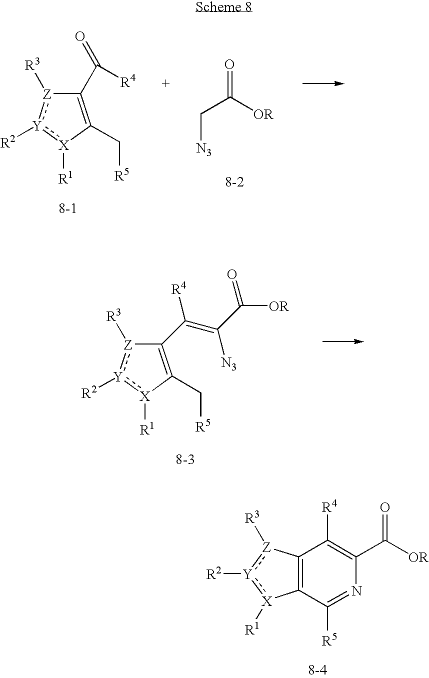

Another general method for formation the desired precursors (R⁵═H, Scheme 9) relies on the condensation of a dicarbonyl compound 9-1 with ethyl glycinate 9-2 (S. Mataka, K. Takahashi, M. Tashiro, J. Heterocyclic. Chem., 1981, 18, 1073-1075, R. P. Kreher, J. Pfister Chemiker-Zeitung , 1984, 9, 275-277) that can provide a mixture of regioisomers 9-3 and 9-4, that can be separated by column chromatography or any other methods known to those skilled in the art.

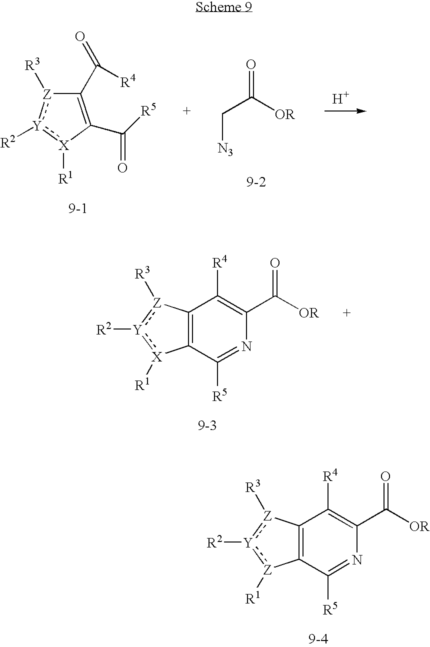

N-Alkylated hydroxylamines can be prepared by various methods described in the literature [for a review see H. J. Wroblowsky in Houben-Weyl, Methoden der Organischen Chemie, Suppl., Vol. E16, Part 1, Thieme, Stuttgart, N.Y., 1990, page 1-96. Scheme 11 describes a method developed by G. Doleschall, Tetrahedron Lett. 1987, 28, 2993-2994, which is based on N-alkylation of 3-methyl 5-hydroxy-4-isoxazole carboxylate 10-1 followed by treatment of 10-2 with hydrochloric acid. Another viable approach relies on the alkylation of bis-t-BOC hydroxylamine 10-4 followed by deprotection of the intermediate 10-5 with hydrochloric acid as described by M. A. Staszak C. W. Doecke, Tetrahedron Lett. 1994, 35, 6021-6024.

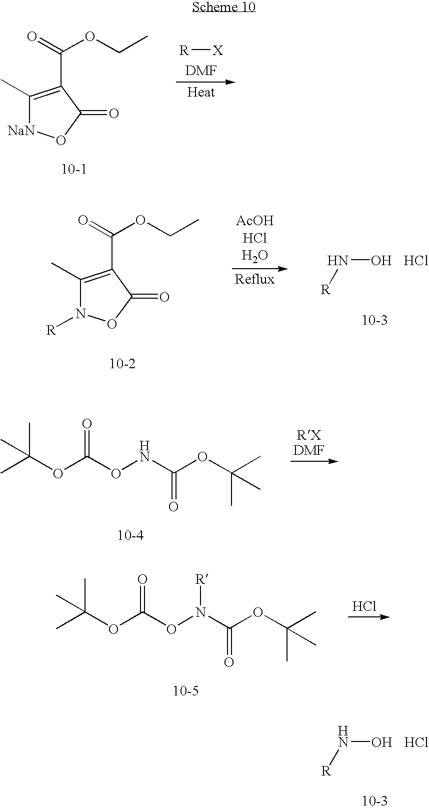

Scheme 11 shows a method for preparation of azaindazole 11-3 and 11-4 from 4-nitro-5-methylpyridine 11-1. Hydrogenation of 11-1 followed by treatment of the intermediate with sodium nitrite in acetic acid can provide azaindazole 11-2. This intermediate can be treated with 4-fluorobenzyl bromide and a base such as potassium carbonate to give both azaindazaole isomers 11-3 and 11-4, which can be separated by chromatography or other methods known to those of ordinary skill in the art. Alternative routes to 5-azaindazoles 11-3 and 11-4 have been described in the literature (Henn, L., J. Chem. Soc. Perkin Trans. 1 1984, 2189; Molina, P., Tetrahedron, 1991, 47, 6737).

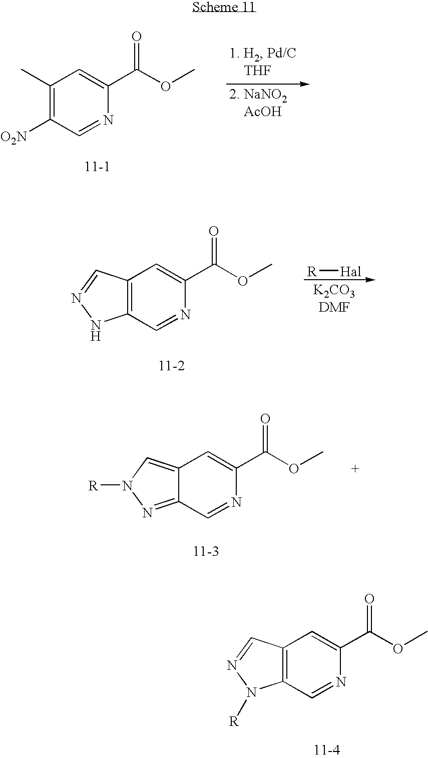

Scheme 12 depicts the synthesis of a 4-substituted azaindole 12-12. Ethyl 2-methyl-1H-pyrrole-3-carboxylate 12-1 (Wee, A. G. H.; Shu, A. Y. L.; Djerassi, C. J. Org. Chem. 1984, 49, 3327-3336) can be treated with a organo halide in the presence of a base such as NaH to provide pyrrole 12-3. Bromination using a bromine source such as NBS followed by radical bromination after the addition of a radical initiator such as benzoyl peroxide can give compound 12-4 which can react with a tosyl glycine ester 12-5 (Ginzel K. D., Brungs, P.; Steckan, E., Tetrahedron, 1989, 45, 1691-1701) to provide 12-6. Cyclization of 12-6 to 12-7 can be effected upon treatment with a base such as lithium hexamethyl disilazide. Catalytic hydrogenolysis (with e.g. Pd/C) can provide ester 12-8. Treatment of 12-8 with an organo halide and a base such as NaH can give 12-9. The hydroxy group in 12-8 can be converted to the triflate 12-10 using trifluoromethanesulfonic anhydride and a base such as triethyl amine. Triflate 12-10 can undergo palladium catalyzed couplings such as the Stille coupling with tributylstannylethene 12-11 in the presence of LiCl (J. K. Stille, Angew. Chem., 1986, 98, 504; Angew. Chem. Int Ed. Engl., 1986, 25, 508; W. J. Scott, J. K. Stille, J. Am. Chem. Soc., 1986, 108, 3033; C. Amatore, A. Jutand, and A. Suarez, J. Am. Chem. Soc., 1993, 115, 9531-9541) using a catalyst such Pd(PPh₃)₂Cl₂ (T. Sakamoto, C. Satoh, Y. Kondo, H. Yamanaka, Chem. Pharm. Bull., 1993, 41, 81-86).

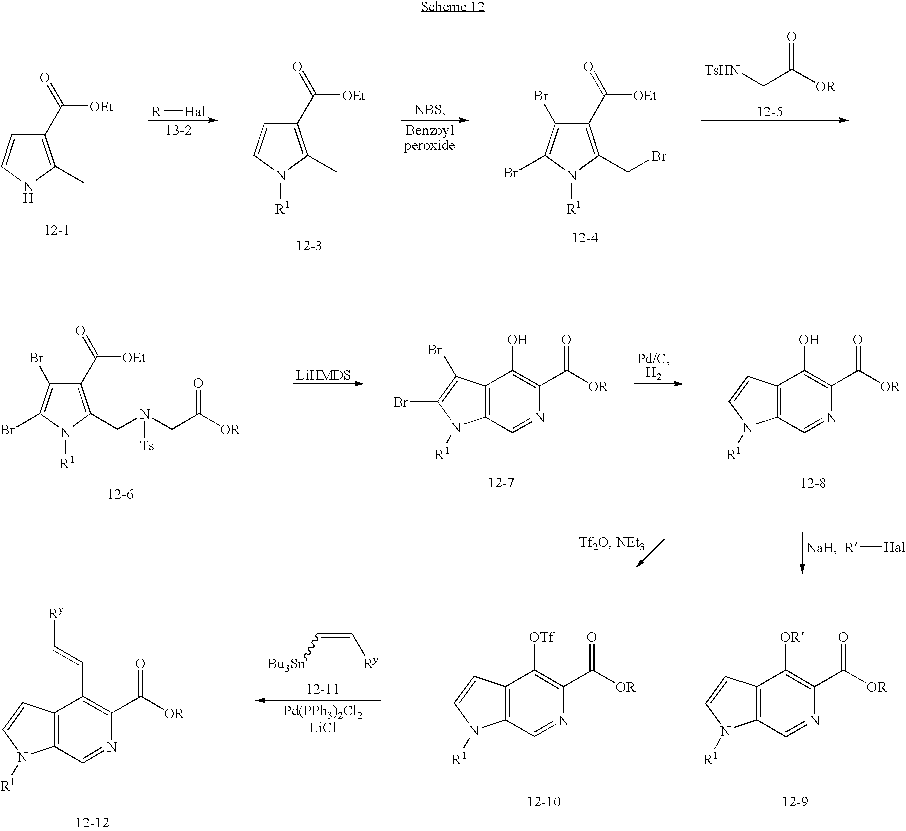

Compounds of formula (I), wherein R³ is —NR⁸C(O)R⁹, —NR⁸S(O)R⁹, or —NR⁸S(O)₂R⁹, wherein R⁸ and R⁹ are as hereinbefore defined, can be prepared by those of ordinary skill in the art by adapting methods found in the chemical literature, as shown in Scheme 13. For example, see MacKenzie, A. R., Tetrahedron, 1986, 42, 3259. The intermediate nitroazaindole can be reduced under conditions known to those of ordinary skill in the art, such as the use of palladium on carbon in the presence of a reducing agent, such as hydrogen, and in a solvent, such as tetrahydrofuran, to give the desired amine. The desired amine, which can be used without isolation from the previous reduction step, may be treated with an acid chloride, acid anhydride, or sulfonyl chloride to provide the corresponding acetamide or sulfonamide. The corresponding ester product can be converted directly to the desired hydroxamates using hydroxylamine or O-alkylated hydroxylamines under basic conditions, or the ester can be cleaved to the corresponding carboxylic acid followed by coupling to give N-alkylated , O-alkylated, or free hydroxamates.

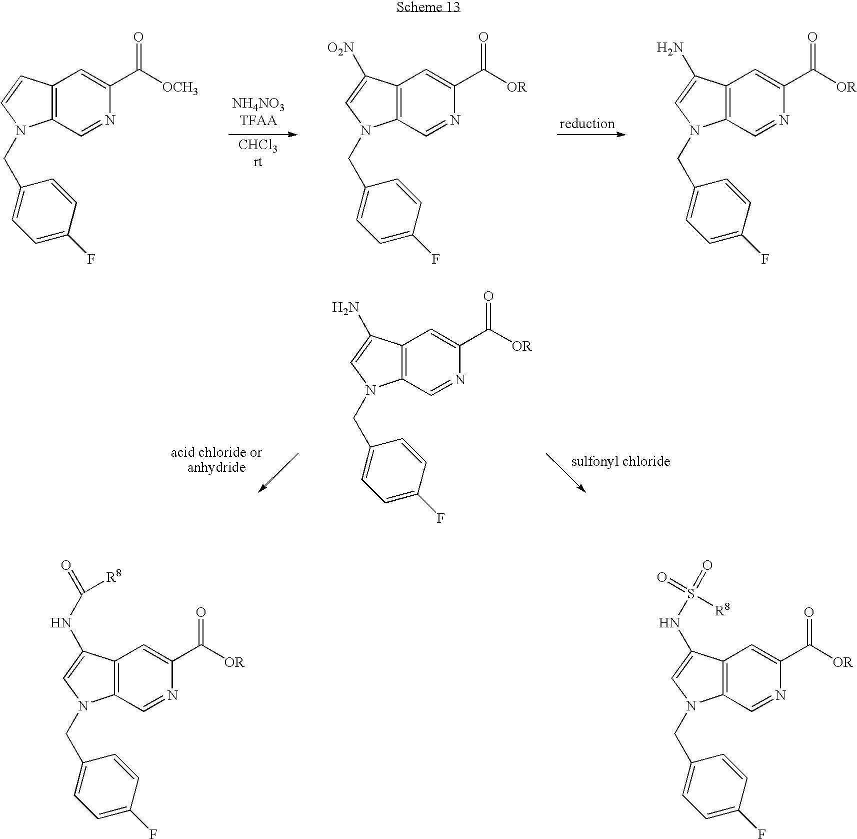

Compounds of formula (I), wherein R³ is —C(O)NR⁸R⁹, wherein R⁸ and R⁹ are as hereinbefore defined, may be prepared from compounds of formula (I), wherein R³ is —CH₂N(alkyl)₂, as shown in Scheme 14. The compound of formula (I), wherein R³ is —CH₂N(alkyl)₂ may be prepared by those of ordinary skill in the art according to procedures described above. These compounds may then be allowed to react with an oxidant, an aqueous solution of potassium permanganate for example, in a solvent, acetone for example, to provide the corresponding carboxylic acid. The acid may then be allowed to react with an appropriate amine of formula HNR⁸R⁹ in the presence of a coupling reagent, such as a carbodiimide, in the presence of a base, N-methylmorpholine for example, and in a solvent, N,N-dimethylformamide for example, to afford the desired amides. The corresponding ester product can be converted directly to the desired hydroxamates using hydroxylamine or O-alkylated hydroxylamines under basic conditions, or the ester can be cleaved to the corresponding carboxylic acid followed by coupling to give N-alkylated , O-alkylated, or free hydroxamates.

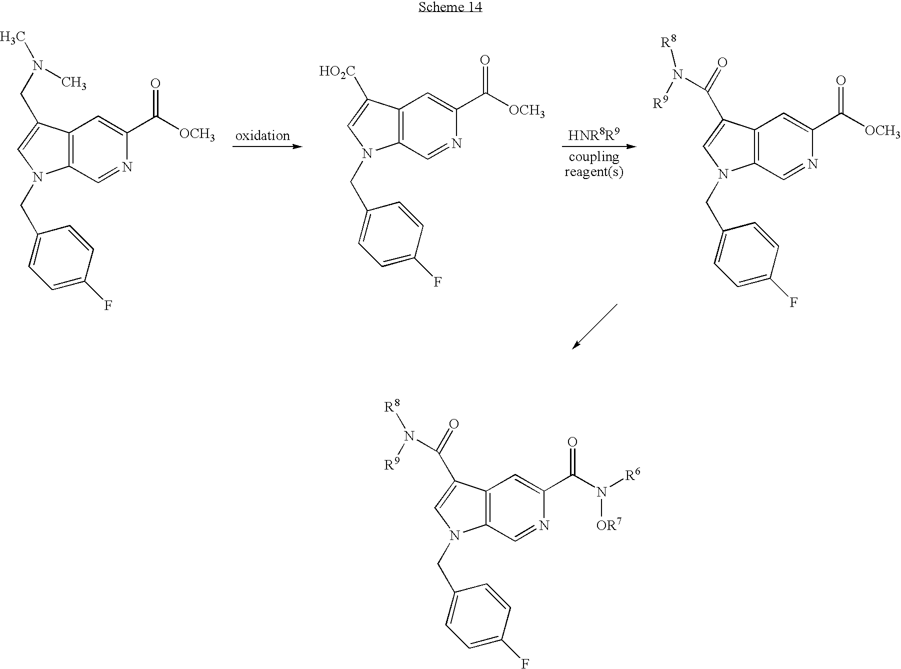

Compounds of formula (I) can be prepared from precursors to compounds of formula (I) containing a carboxylic acid ester group at the 5-position of the azaindole ring system. Such a transformation can be accomplished by first converting the ester to the corresponding carboxylic acid by reaction with a base, lithium or sodium hydroxide for example, in a solvent, methanol for example, and at a temperature from about room temperature to about 100° C., about 50° C. for example.

The corresponding carboxylic acid can then be allowed to react with hydroxylamine, an N-alkylhydroxylamine, an O-alkylhydroxylamine, or an N,O-dialkylhydroxylamine. Such a reaction may be conducted in the presence of a coupling agent or combination of coupling agents, a carbodiimide for example, and in the presence of a base, an amine for example, to provide the desired compounds of formula (I).

### EXAMPLES

The examples below are intended only to illustrate particular embodiments of the present invention and are not meant to limit the scope of the invention in any manner.

In the examples described below, unless otherwise indicated, all temperatures in the following description are in degrees Celsius (° C.) and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromatography (HPLC) or thin-layer chromatography (TLC) and terminated as judged by the consumption of starting material. The TLC plates were visualized by UV, phosphomolybdic acid stain, or iodine stain.

Unless otherwise indicated, ¹H-NMR spectra were recorded on a Bruker instrument operating at 300 MHz and ¹³C-NMR spectra were recorded at 75 MHz. NMR spectra were obtained as DMSO-d6 or CDCl₃ solutions (reported in ppm), using chloroform as the reference standard (7.25 ppm and 77.00 ppm) or DMSO-d6 ((2.50 ppm and 39.52 ppm)). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCI. All melting points are uncorrected.

All final products had greater than 95% purity (by HPLC at wavelengths of 220 nm and 254 nm).

All elemental analyses for compounds herein, unless otherwise specified, provided values for C, H, and N analysis that were within 0.4% of the theoretical value, and are reported as “C, H, N.”

In the following examples and preparations, “LDA” means lithium diisopropyl amide, “Et” means ethyl, “Ac” means acetyl, “Me” means methyl, “Ph” means phenyl, (PhO)₂POCl means chlorodiphenylphosphate, “HCl” means hydrochloric acid, “EtOAc” means ethyl acetate, “Na₂CO₃” means sodium carbonate, “NaOH” means sodium hydroxide, “NaCl” means sodium chloride, “NEt₃” means triethylamine , “THF” means tetrahydrofuran, “DIC” means diisopropylcarbodiimide, “HOBt” means hydroxy benzotriazole, “H₂O” means water, “NaHCO₃” means sodium hydrogen carbonate, “K₂CO₃” means potassium carbonate, “MeOH” means methanol, “i-PrOAc” means isopropyl acetate, “MgSO₄” means magnesium sulfate, “DMSO” means dimethylsulfoxide, “AcCl” means acetyl chloride, “CH₂Cl₂” means methylene chloride, “MTBE” means methyl t-butyl ether, “DMF” means dimethyl formamide, “SOCl₂” means thionyl chloride, “H₃PO₄” means phosphoric acid, “CH₃SO₃H” means methanesulfonic acid, “Ac₂O” means acetic anhydride, “CH₃CN” means acetonitrile, and “KOH” means potassium hydroxide.

### Example A

### Methyl 1-(4-fluorobenzyl)-3-nitro-1H-pyrrolo[2,3-c]pyridine-5-carboxylate

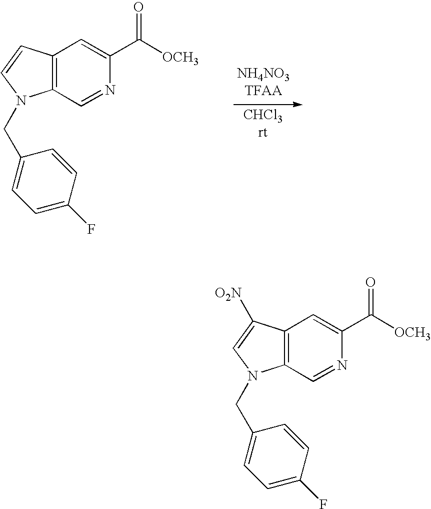

To a stirred solution of the azaindole methyl ester (3.53 g, 12.42 mmol) in chloroform (100 mL) was added trifluoroacetic anhydride (35 mL, 248.3 mmol) followed by addition of 1 eq. portions of ammonium nitrate (4.94 g, 62.1 mmol). The ammonium nitrate was added every 2 h until the reaction was judged to be complete by LCMS. The reaction mixture was quenched by pouring it into saturated sodium bicarbonate solution and the mixture was extracted with dichloromethane. The combined organic extracts were dried over sodium sulfate, filtered, and concentrated to give a yellowish-brown solid (4.20 g, 100%), which was carried on to the next step without further purification.

### Example B

### Methyl 1-(4-fluorobenzyl)-3-amino-1H-pyrrolo[2,3-c]pyridine-5-carboxylate

A solution of 3-nitro-1-(4-fluoro-benzyl)-1H-pyrrolo[2,3-c]pyridine-5-carboxylic acid methyl ester (10.0 g, 30.4 mmol) in dry THF (fresh bottle, 0.5 L) was thoroughly flushed with nitrogen. Under nitrogen atmosphere, Pd/C (10%, 1.0 g) was added. Hydrogen was bubbled through the solution for 1 hour after which the reaction flask was left stirring with a hydrogen balloon for 2 days. The mixture was then filtered through a double paper filter under nitrogen atmosphere and fresh Pd/C (1.0 g) was added. Hydrogen was bubbled through the solution for 1 hour after which the reaction flask was left stirring with a hydrogen balloon for 4 days. Then, the mixture was filtered through a double paper filter under nitrogen atmosphere and evaporated in vacuo to give crude methyl 1-(4-fluorobenzyl)-3-amino-1H-pyrrolo[2,3-c]pyridine-5-carboxylate as an oil, which was used without further purification.

### Example C

### Reaction of Methyl 1-(4-fluorobenzyl)-3-amino-1H-pyrrolo[2,3-c]pyridine-5-carboxylate with Acid Chlorides, Acid Anhydrides, or Sulfonyl Chlorides

Crude methyl 1-(4-fluorobenzyl)-3-amino-1H-pyrrolo[2,3-c]pyridine-5-carboxylate

(28.1 mmol) was dissolved in dry THF (0.11 M) under argon atmosphere. The acid chloride, acid anhydride, or sulfonyl chloride (1.17 eq) and DIPEA (1.40 eq) were added and the resulting mixture was stirred for 18 h. The solvent was evaporated in vacuo to give crude product an as oil. The oil was further purified by reverse-phase flash column chromatography using RP-silica and a solvent gradient from acetronitrile:water (1:4 to 1:1), to provide the desired product as a solid.

### Example D

### Preparation of 1-(4-fluorobenzyl)-5-(methoxycarbonyl)-1H-pyrrolo[2,3-c]pyridine-3-carboxylic Acid

To a solution of 3-dimethylaminomethyl-1-(4-fluorobenzyl)-1H-pyrrolo[2,3-c]pyridine-5-carboxylic acid methyl ester (6.29 g, 17.6 mmol) in acetone (125 mL) was added dropwise a solution of potassium permanganate (28.9 g, 0.183 mmol) in water (550 mL) over 2 h. After 3 h, the reaction mixture was filtered through celite and the residue was washed with water. The combined filtrate and washings was acidified to pH 5 with concentrated hydrochloric acid. The formed precipitate was filtered, washed with water, and dried in a vacuum oven at 40° C. overnight to give 1-(4-fluorobenzyl)-5-(methoxycarbonyl)-1H-pyrrolo[2,3-c]pyridine-3-carboxylic acid (3.22 g) as a light red solid.

### Example E

### Reaction of 1-(4-fluorobenzyl)-5-(methoxycarbonyl)-1H-pyrrolo[2,3-c]pyridine-3-carboxylic Acid with Amines

Under argon atmosphere, NMM (1.2 eq) and CDMT (1.2 eq) were added to a suspension of 1-(4-fluorobenzyl)-5-(methoxycarbonyl)-1H-pyrrolo[2,3-c]pyridine-3-carboxylic acid (9.57 mmol, 1.0 eq) in dry DMF (0.48 M). After 1.5 h, the appropriate amine (1.5 eq) was added and stirring was continued for 22 h. Water was added and the resulting mixture was extracted with ethyl acetate four times, washed with brine, dried over sodium sulfate and concentrated under reduced pressure. The residue was further purified by column chromatography (silica gel, dichloromethane/methanol 98:2 to 6:1) to afford the desired amide.

### Example F

### Reaction of Methyl 3-(chlorosulfonyl)-1-(4-fluorobenzyl)-1H-pyrrolo[2,3-c]pyridine-5-carboxylate with Amines

Preparation of methyl 3-(chlorosulfonyl)-1-(4-fluorobenzyl)-1H-pyrrolo[2,3-c]pyridine-5-carboxylate. To a stirring solution of methyl 1-(4-fluorobenzyl)-1H-pyrrolo[2,3-c]pyridine-5-carboxylate (1 g, 3.4 mmol) in chlorosulfonic acid (6 mL) was added sulfuryl dichloride (3 mL, 34 mmol) slowly. After stirring overnight at room temperature, the solution was added dropwise to ice and the resulted white precipitate was filtered and dried in vacuo.

To a solution of 3-chlorosulfonyl-1-(4-fluorobenzyl)-1H-pyrrolo[2,3-c]pyridine-5-carboxylic acid methyl ester (2.49 g, 6.53 mmol) in dichloromethane (40 mL) was added DIPEA (1.370 mL, 7.84 mmol) and the appropriate amine (1 to 2 eq. per equivalent of sulfonyl chloride) under an argon atmosphere. After 18 hours of stirring, citric acid (aq., 10%, 30 mL) was added. The layers were separated and the aqueous phase extracted with DCM (2×40 mL). The combined organic layers were washed with brine (20 mL), dried with phase separator and evaporated in vacuo to give the desired products.

### Example G

### Preparation of Hydroxamates from Carboxylic Acid Esters

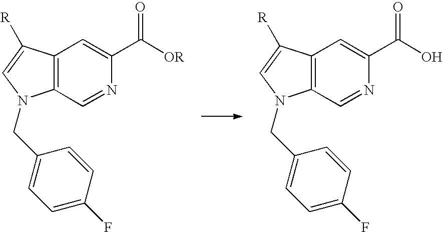

To a solution of the appropriate carboxylic acid ester (1.0 eq) in methanol (0.05 M) was added aqueous lithium hydroxide solution (3 M, 3.0 eq). The mixture was heated to 50° C. for 2 days. Additional portions of lithium hydroxide may be used if necessary. The resultant mixture was acidified to pH 5 with 1 M aqueous HCl and the solvent was evaporated in vacuo. The residue was stirred in acetone for 2 days. The resulting solids were filtered and dried to provide the desired carboxylic acid, which was used in the next step without further purification.

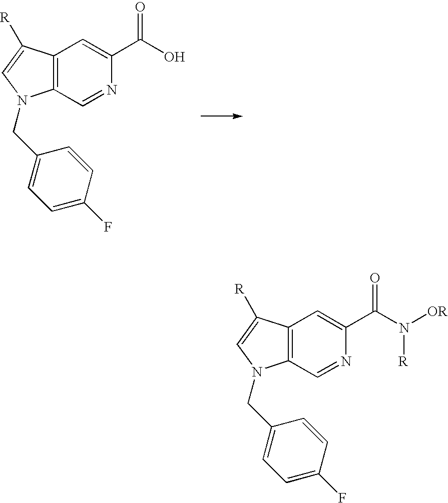

Under argon atmosphere, NMM (1.2 eq) and CDMT (1.2 eq) were added to a solution of the appropriate carboxylic acid (3.61 mmol) in dry DMF (0.36 M). After 1.5 h, hydroxylamine, an N-alkylhydroxylamine, an O-alkylhydroxylamine, or an N,O-dialkylhydroxylamine (10.0 eq) was added and stirring was continued for about 21 h. Brine and water were added and the resulting mixture was extracted with ethyl acetate three times. The combined organic layers were washed with brine, dried over sodium sulfate and evaporated in vacuo. The residue was optionally further purified by crystallization from an appropriate solvent (ethanol, 2-propanol, diisopropyl ether, or a mixture thereof) or by column chromatography (silica gel), or combination of both, to afford the desired product.

### Examples 1 to 57

| Ex- | | | |
|---|---|---|---|
| am- | | | |
| ple | | | |
| No. | Structure | Name | ¹H NMR |
| 1 | 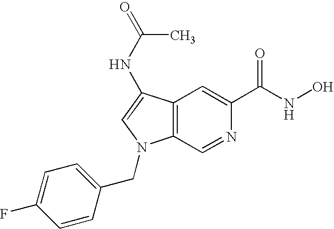 | 3-(acetylamino)-1-(4- fluorobenzyl)-N-hydroxy- 1H-pyrrolo[2,3-c]pyridine- 5-carboxamide | (CD₃OD, 400 MHz) d 8.68 (1 H, s), 8.45 (1 H, d), 8.05 (1 H, s), 7.23 (2 H, t), 7.04 (1 H, t), 5.49 (2 H, s), 2.20 (3 H, s) |
| 2 | 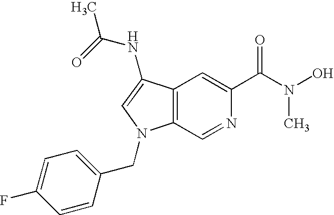 | 3-(acetylamino)-1-(4- fluorobenzyl)-N-hydroxy-N- methyl-1H-pyrrolo[2,3- c]pyridine-5-carboxamide | (CD₃OD, 400 MHz) d 8.73 (1 H, s), 8.53 (1 H, b), 8.14 (1 H, s), 7.28 (2 H, t), 7.04 (1 H, t), 5.51 (2 H, s), 3.43 (3 H, s), 2.20 (3 H, s) |
| 3 | 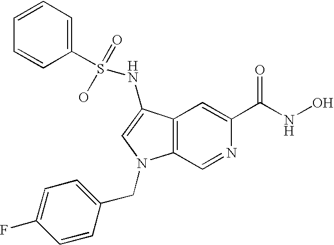 | 1-(4-fluorobenzyl)-N- hydroxy-3- [(phenylsulfonyl)amino]- 1H-pyrrolo[2,3-c]pyridine- 5-carboxamide | (CD₃OD, 400 MHz) d 8.61 (1 H, s), 7.93 (1 H, s), 7.64 (2 H, d), 7.49 (1 H, m), 7.41 (1 H, s), 7.36 (2 H, m), 7.14 (2 H, m), 7.06 (2 H, t), 5.44 (2 H, s) |
| 4 | 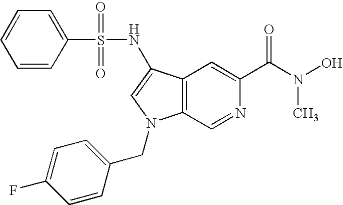 | 1-(4-fluorobenzyl)-N- hydroxy-N-methyl-3- [(phenylsulfonyl)amino]- 1H-pyrrolo[2,3-c]pyridine- 5-carboxamide | (CD₃OD, 400 MHz) d 8.98 (1 H, s), 7.58 (1 H, b), 7.76 (1 H, s), 7.53 (2 H, d), 7.46 (1 H, m), 7.30 (2 H, t), 7.12 (2 H, m), 7.06 (2 H, t), 5.49 (2 H, s), 3.36 (3 H, s) |
| 5 | 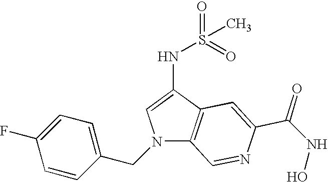 | 1-(4-fluorobenzyl)-N- hydroxy-3- [(methylsulfonyl)amino]- 1H-pyrrolo[2,3-c]pyridine- 5-carboxamide | (CD₃OD, 400 MHz) d 8.72 (1 H, s), 8.36 (1 H, s), 7.68 (1 H, s), 7.28 (2 H, m), 7.06 (2 H, t), 5.53 (2 H, s), 2.94 (3 H, s) |
| 6 | 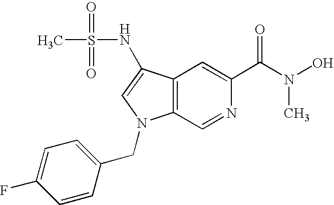 | 1-(4-fluorobenzyl)-N- hydroxy-N-methyl-3- [(methylsulfonyl)amino]- 1H-pyrrolo[2,3-c]pyridine- 5-carboxamide | (CD₃OD, 400 MHz) d 8.77 (1 H, s), 8.25 (1 H, b), 7.77 (1 H, s), 7.32 (2 H, m), 7.08 (2 H, t), 5.56 (2 H, s), 3.43 (3 H, s), 2.95 (3 H, s) |
| 7 | 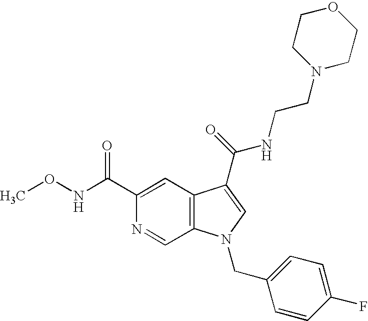 | 1-(4-fluorobenzyl)-N~5~- methoxy-N~3~-(2- morpholin-4-ylethyl)-1H- pyrrolo[2,3-c]pyridine-3,5- dicarboxamide | (CD₃OD, 400 MHz) d 8.90 (2 H, d), 8.16 (1 H, s), 7.32 (2 H, t), 7.09 (2 H, t), 5.58 (2 H, s), 3.82 (3 H, s), 3.74 (4 H, t), 3.64 (2 H, t), 2.67 (2 H, t), 2.57 (4 H, m) |
| 8 | 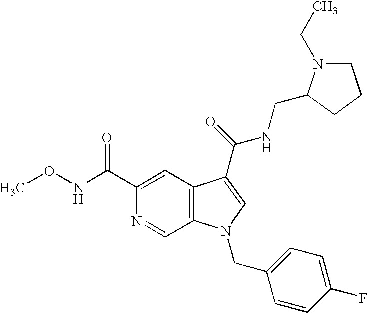 | N3~-[(1-ethylpyrrolidin-2- yl)methyl]-1-(4- fluorobenzyl)-N~5~- methoxy-1H-pyrrolo[2,3- c]pyridine-3,5- dicarboxamide | (CD₃OD, 400 MHZ) d 8.90 (2 H, d), 8.30 (1 H, s), 7.35 (2 H, t), 7.11 (2 H, t), 5.62 (2 H, s), 3.84 (3 H, s), 3.66 (2 H, m), 3.64 (3 H, m), 3.20 (2 H, m), 2.30 (1 H, b), 2.20 (1 H, b), 2.0 (2 H, b), 1.43 (3 H, t) |
| 9 | 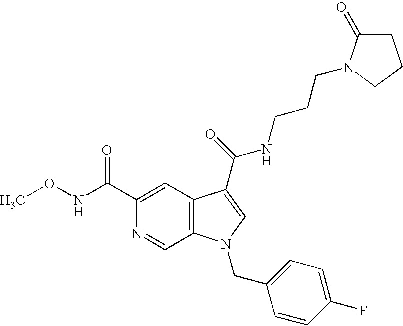 | 1-(4-fluorobenzyl)-N~5~- methoxy-N~3~-[3-(2- oxopyrrolidin-1-yl)propyl]- 1H-pyrrolo[2,3-c]pyridine- 3,5-dicarboxamide | (CD₃OD, 400 MHz) d 8.82 (1 H, s), 8.75 (1 H, s), 8.16 (1 H, s), 7.31 (2 H, t), 7.08 (2 H, t), 5.57 (2 H, s), 3.82 (3 H, s), 3.47 (2 H, t), 3.39 (4 H, m), 2.38 (2 H, t), 2.04 (2 H, m), 1.85 (2 H, m) |
| 10 | 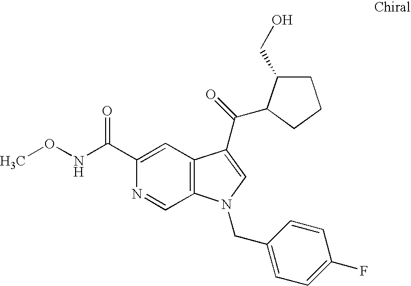 | 1-(4-fluorobenzyl)-3-{[(2S)- 2-(hydroxymethyl)pyrrolidin- 1-yl]carbonyl}-N-methoxy- 1H-pyrrolo[2,3-c]pyridine- 5-carboxamide | (CD₃OD, 400 MHz) d 8.66 (1 H, s), 8.62 (1 H, s), 8.09 (1 H, s), 7.21 (2 H, t), 6.99 (2 H, t), 5.52 (2 H, s), 4.31 (1 H, b), 3.73 (3 H, s), 3.65 (5 H, m), 1.99 (4 H, m) |
| 11 | 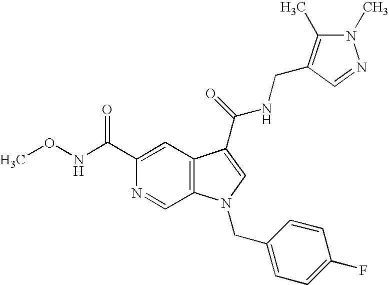 | N~3~-[(1,5-dimethyl-1H- pyrazol-4-yl)methyl]-1-(4- fluorobenzyl)-N~5~- methoxy-1H-pyrrolo[2,3- c]pyridine-3,5- dicarboxamide | (CD₃OD, 400 MHz) d 8.83 (1 H, s), 8.77 (1 H, s), 8.13 (1 H, s), 7.41 (1 H, s), 7.30 (2 H, t), 7.28 (2 H, t), 5.55 (2 H, s), 4.39 (2 H, s), 3.82 (3 H, s), 3.75 (3 H, s), 2.33 (3 H, s) |
| 12 | 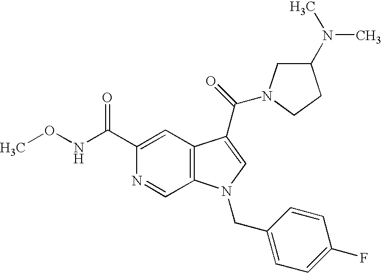 | 3-{[3- (dimethylamino)pyrrolidin- 1-yl]carbonyl)-1-(4- fluorobenzyl)-N-methoxy- 1H-pyrrolo[2,3-c]pyridine- 5-carboxamide | (CD₃OD, 400 MHz) d 8.75 (2 H, s) 8.13 (1 H, s), 7.30 (2 H, t), 7.08 (2 H, t), 5.61 (2 H, s), 4.00 (2 H, b), 3.82 (3 H, s), 3.55 (2 H, b), 2.38 (6 H, b), 2.25 (1 H, b), 1.85 (2 H, b), some peaks are difficult to resolve. |
| 13 | 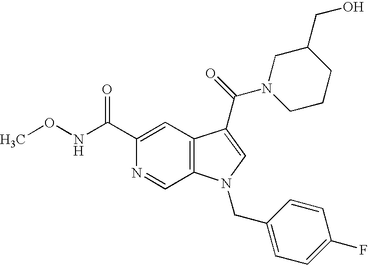 | 1-(4-fluorobenzyi)-3-{[3- (hydroxymethyl)piperidin- 1-yl]carbonyl}-N-methoxy- 1H-pyrrolo[2,3-c]pyridine- 5-carboxamide | (CD₃OD, 400 MHz) d 8.69 (1 H, s), 8.33 (1 H, s), 7.94 (1 H, s), 7.22 (2 H, t), 7.00 (2 H, t), 5.51 (2 H, s), 4.25 (1 H, b), 3.74 (3 H, s), 3.39 (2 H, m), 3.25 (2 H, s), 3.05 (1 H, b), 2.90 (1 H, b), 1.75 (3 H, b), 1.5 (1 H, b), 1.29 (1 H, b) |
| 14 | 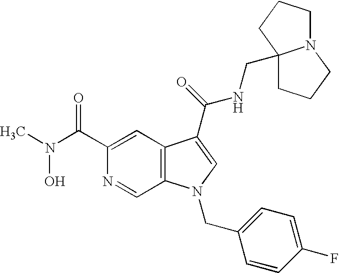 | 1-(4-fluorobenzyl)-N~5~- hydroxy-N~5~-methyl- N~3~-(tetrahydro-1H- pyrrolizin-7a(5H)- ylmethyl)-1H-pyrrolo[2,3- c]pyridine-3,5- dicarboxamide | (CD₃OD, 400 MHz) d 8.90 (1 H, s), 8.42 (1 H, s), 8.36 (1 H, s), 7.43 (2 H, t), 7.17 (2 H, t), 5.66 (2 H, s), 3.74 (2 H, s), 3.62 (2 H, m), 3.23 (2 H, m), 3.20 (3 H, s), 2.19 (4 H, m), 2.07 (4 H, m) |
| 15 | 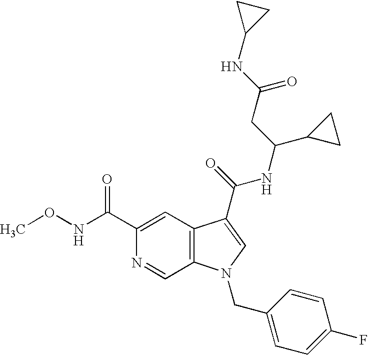 | N~3~-[1-cyclopropyl-3- (cyclopropylamino)-3- oxopropyl]-1-(4- fluorobenzyl)-N~5~- methoxy-1H-pyrrolo[2,3- c]pyridine-3,5- dicarboxamide | (CD₃OD, 400 MHz) d 8.73 (1 H, s), 8.70 (1 H, s), 8.10 (1 H, s), 7.24 (2 H, t), 7.01 (2 H, t), 5.50 (2 H, s), 3.74 (3 H, s), 3.62 (1 H, m), 2.50 (3 H, m), 1.00 (1 H, b), 0.56 (2 H, m), 0.45 (2 H, b), 0.35 (4 H, m) |
| 16 | 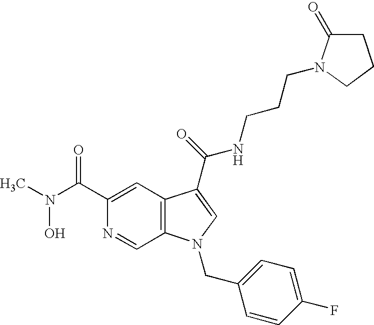 | 1-(4-fluorobenzyl)-N~5~- hydroxy-N~5~-methyl- N~3~-[3-(2-oxopyrrolidin- 1-yl)propyl]-1H-pyrrolo[2,3- c]pyridine-3,5- dicarboxamide | (CD₃OD, 400 MHz) d 8.81 (1 H, s), 8.62 (1 H, s), 8.23 (1 H, s), 7.34 (2 H, t), 7.09 (2 H, t), 5.59 (2 H, s), 3.50 (2 H, t), 3.41 (3 H, s), 3.39 (4 H, m), 2.39 (2 H, t), 2.07 (2 H, m), 1.84 (2 H, m) |
| 17 | 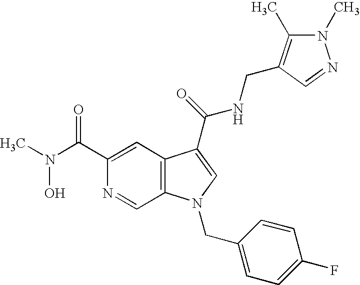 | N~3~-[(1,5-dimethyl-1H- pyrazol-4-yl)methyl]-1-(4- fluorobenzyl)-N~5~- hydroxy-N~5~-methyl-1H- pyrrolo[2,3-c]pyridine-3,5- dicarboxamide | (CD₃OD, 400 MHz) d 8.80 (1 H, s), 8.62 (1 H, b), 8.18 (1 H, s), 7.40 (1 H, s), 7.31 (2 H, t), 7.08 (2 H, t), 5.56 (2 H, s), 4.37 (2 H, s), 3.74 (3 H, s), 3.41 (3 H, s), 2.31 (3 H, s) |
| 18 | 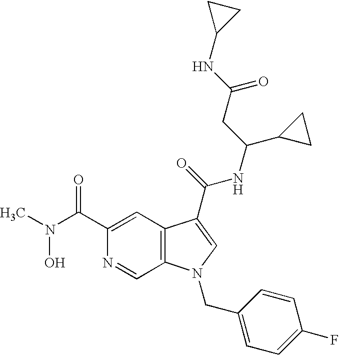 | N~3~-[1-cyclopropyl-3- (cyclopropylamino)-3- oxopropyl]-1-(4- fluorobenzyl)-N~5~- hydroxy-N~5~-methyl-1H- pyrrolo[2,3-c]pyridine-3,5- dicarboxamide | (CD₃OD, 400 MHz) d 8.82 (1 H, s), 8.65 (1 H, b), 8.24 (1 H, s), 7.34 (2 H, t), 7.10 (2 H, t), 5.59 (2 H, s), 3.70 (1 H, m), 3.42 (3 H, s), 2.60 (3 H, m), 1.05 (1 H, b), 0.66 (2 H, m), 0.55 (2 H, b), 0.43 (4 H, m) |
| 19 | 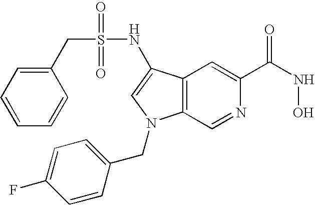 | 3-[(benzylsulfonyl)amino]- 1-(4-fluorobenzyl)-N- hydroxy-1H-pyrrolo[2,3- c]pyridine-5-carboxamide | (CD₃OD, 400 MHz) d 8.92 (1 H, s), 8.38 (1 H, s), 7.68 (1 H, s), 7.31 (5 H, m), 7.23 (2 H, m), 7.10 (2 H, t), 5.55 (2 H, s), 4.42 (3 H, s) |
| 20 | 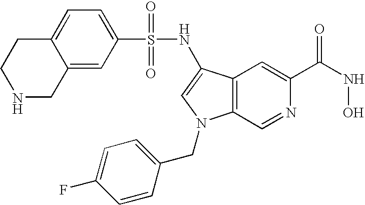 | 1-(4-fluorobenzyl)-N- hydroxy-3-[(1,2,3,4- tetrahydroisoquinolin-7- ylsulfonyl)amino]-1H- pyrrolo[2,3-c]pyridine-5- carboxamide | (CD₃OD, 400 MHz) d 8.72 (1 H, s), 7.74 (2 H, s), 7.51 (1 H, s), 7.26 (1 H, d), 7.25 (2 H, m), 7.08 (3 H, m), 5.52 (2 H, s), 4.40 (2 H, s), 3.50 (2 H, t), 3.06 (2 H, t) |
| 21 | 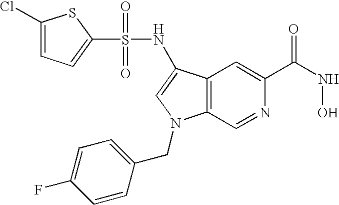 | 3-{[(5-chloro-2- thienyl)sulfonyl]amino}-1- (4-fluorobenzyl)-N- hydroxy-1H-pyrrolo[2,3- c]pyridine-5-carboxamide | (CD₃OD, 400 MHz) d 8.86 (1 H, s), 8.20 (1 H, s), 7.64 (1 H, s), 7.16 (3 H, m), 7.01 (2 H, t), 6.85 (2 H, d), 5.48 (2 H, s) |
| 22 | 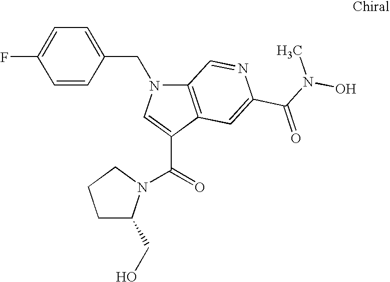 | 1-(4-fluorobenzyl)-N- hydroxy-3-{[(2S)-2- (hydroxymethyl)pyrrolidin- 1-yl]carbonyl}-N-methyl- 1H-pyrrolo[2,3-c]pyridine- 5-carboxamide | (CD₃OD, 400 MHz) d 8.78 (1 H, s), 8.65 (1 H, b), 8.24 (1 H, s), 7.34 (2 H, t), 7.10 (2 H, t), 5.61 (2 H, s), 4.39 (1 H, b), 3.74 (4 H, b), 3.41 (3 H, s), 2.09 (4 H, b) |
| 23 | 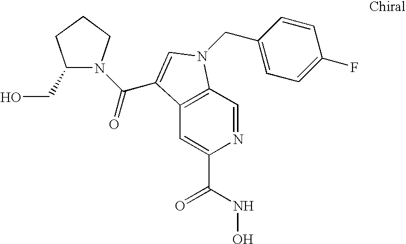 | 1-(4-fluorobenzyl)-N- hydroxy-3-{[(2S)-2- (hydroxymethyl)pyrrolidin- 1-yl]carbonyl}-1H- pyrrolo[2,3-c]pyridine-5- carboxamide | (CD₃OD, 400 MHz) d 8.73 (1 H, s), 8.70 (1 H, b), 8.15 (1 H, s), 7.29 (2 H, t), 7.06 (2 H, t), 5.59 (2 H, s), 4.39 (1 H, b), 3.77 (4 H, b), 2.01 (4 H, b) |
| 24 | 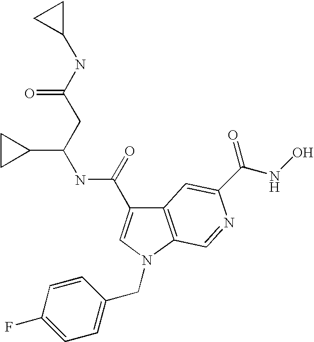 | N~3~-[1-cyclopropyl-3- (cyclopropylamino)-3- oxopropyl]-1-(4- fluorobenzyl)-N~5~- hydroxy-1H-pyrrolo[2,3- c]pyridine-3,5- dicarboxamide | (CD₃OD, 400 MHz) d 8.70 (2 H, d), 8.09 (1 H, s), 7.23 (2 H, t), 7.01 (2 H, t), 5.49 (2 H, s), 3.62 (1 H, m), 2.49 (3 H, m), 0.97 (1 H, b), 0.55 (2 H, m), 0.40 (2 H, b), 0.33 (4 H, m) |
| 25 | 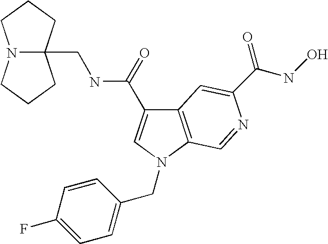 | 1-(4-fluorobenzyl)-N~5~- hydroxy-N~3~-(tetrahydro- 1H-pyrrolizin-7a(5H)- ylmethyl)-1H-pyrrolo[2,3- c]pyridine-3,5- dicarboxamide | (CD₃OD, 400 MHz) d 8.85 (1 H, s), 8.83 (1 H, s), 8.24 (1 H, s), 7.35 (2 H, t), 7.11 (2 H, t), 5.61 (2 H, s), 3.68 (2 H, s), 3.57 (2 H, m), 3.15 (2 H, m), 2.14 (4 H, m), 2.02 (4 H, m) |
| 26 | 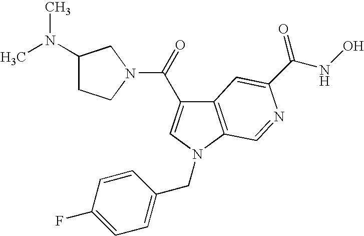 | 3-{[3- (dimethylamino)pyrrolidin- 1-yl]carbonyl}-1-(4- fluorobenzyl)-N-hydroxy- 1H-pyrrolo[2,3-c]pyridine- 5-carboxamide | (CD₃OD, 400 MHz) d 8.72 (1 H, s), 8.71 (1 H, s), 8.16 (1 H, s), 7.30 (2 H, t), 7.06 (2 H, t), 5.59 (2 H, s), 3.97 (2 H, b), 3.53 (1 H, b), 3.02 (1 H, b), 2.39 (6 H, m), 2.30 (1 H, b), 1.92 (2 H, b) |
| 27 | 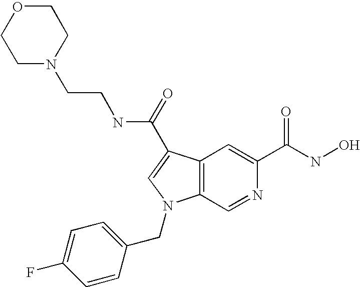 | 1-(4-fluorobenzyl)-N~5~- hydroxy-N~3~-(2- morpholin-4-ylethyl)-1H- pyrrolo[2,3-c]pyridine-3,5- dicarboxamide | (CD₃OD, 400 MHz) d 8.77 (2 H, s), 8.14 (1 H, s), 7.30 (2 H, t), 7.08 (2 H, t), 5.57 (2 H, s), 3.71 (4 H, t), 3.55 (2 H, t), 2.64 (2 H, t), 2.56 (4 H, t) |
| 28 | 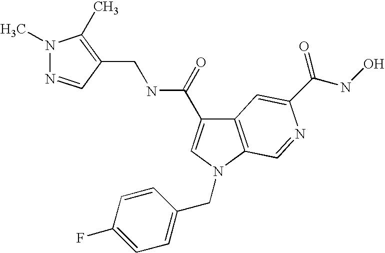 | N~3~-[(1,5-dimethyl-1H- pyrazol-4-yl)methyl]-1-(4- fluorobenzyl)-N~5~- hydroxy-1H-pyrrolo[2,3- c]pyridine-3,5- dicarboxamide | (CD₃OD, 400 MHz) d 8.80 (1 H, s), 8.76 (1 H, b), 8.12 (1 H, s), 7.40 (1 H, s), 7.31 (2 H, t), 7.08 (2 H, t), 5.54 (2 H, s), 4.38 (2 H, s), 3.74 (3 H, s), 2.33 (3 H, s) |
| 29 | 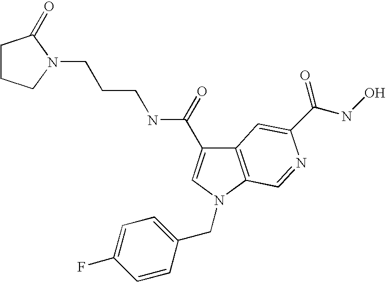 | 1-(4-fluorobenzyl)-N~5~- hydroxy-N~3~-[3-(2- oxopyrrolidin-1-yl)propyl]- 1H-pyrrolo[2,3-c]pyridine- 3,5-dicarboxamide | (CD₃OD, 400 MHz) d 8.80 (1 H, s), 8.76 (1 H, b), 8.16 (1 H, s), 7.31 (2 H, t), 7.08 (2 H, t), 5.58 (2 H, s), 3.50 (2 H, t), 3.38 (4 H, m), 2.39 (2 H, t), 2.02 (2 H, m), 1.86 (2 H, m) |
| 30 | 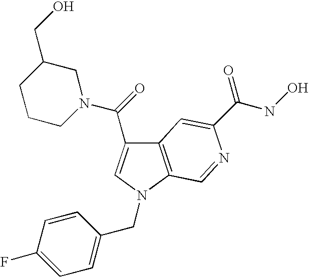 | 1-(4-fluorobenzyl)-N- hydroxy-3-{[3- (hydroxymethyl)piperidin- 1-yl]carbonyl}-1H- pyrrolo[2,3-c]pyridine-5- carboxamide | (CD₃OD, 400 MHz) d 8.76 (1 H, s), 8.38 (1 H, s), 8.01 (1 H, s), 7.31 (2 H, t), 7.08 (2 H, t), 5.58 (2 H, s), 4.30 (2 H, b), 3.48 (1 H, m), 3.40 (1 H, m), 3.15 (1 H, b), 2.95 (1 H, b), 1.86 (3 H, b), 1.62 (1 H, b), 1.36 (1 H, b) |
| 31 | 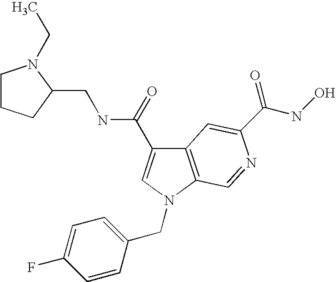 | N~3~-[(1-ethylpyrrolidin-2- yl)methyl]-1-(4- fluorobenzyl)-N~5~- hydroxy-1H-pryyolo[2,3- c]pyridine-3,5- dicarboxamide | (CD₃OD, 400 MHz) d 8.80 (1 H, s), 8.79 (1 H, s), 8.17 (1 H, s), 7.31 (2 H, t), 7.08 (2 H, t), 5.58 (2 H, s), 3.66 (1 H, dd), 3.47 (2 H, m), 3.13 (1 H, m), 2.97 (1 H, b), 2.51 (2 H, b), 2.07 (1 H, b), 1.87 (3 H, b), 1.24 (3 H, t) |
| 32 | 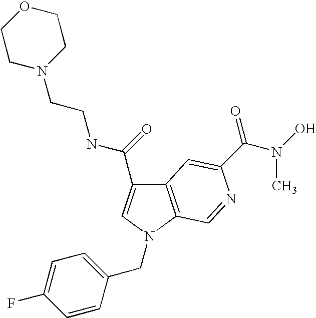 | 1-(4-fluorobenzyl)-N~5~- hydroxy-N~5~-methyl- N~3~-(2-morpholin-4- ylethyl)-1H-pyrrolo[2,3- c]pyridine-3,5- dicarboxamide | (CD₃OD, 400 MHz) d 8.80 (1 H, d), 8.30 (1 H, s), 8.20 (1 H, d), 7.31 (2 H, t), 7.08 (2 H, t), 5.58 (2 H, s), 3.72 (4 H, b), 3.56 (2 H, t), 3.42 (3 H, s), 2.67 (2 H, t), 2.61 (4 H, b) |
| 33 | 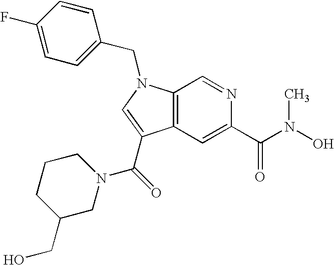 | 1-(4-fluorobenzyl)-N- hydroxy-3-{[3- (hydroxymethy[)piperidin- 1-yl]carbonyl}-N-methyl- 1H-pyrrolo[2,3-c]pyridine- 5-carboxamide | (CD₃OD, 400 MHz) d 8.79 (1 H, s), 8.20 (1 H, b), 8.09 (1 H, s), 7.34 (2 H, t), 7,08 (2 H, t), 5.60 (2 H, s), 4.62 (4 H, b), 3.48 (2 H, m), 3.41 (3 H, s), 1.89 (1 H, b), 1.77 (2 H, b), 1.57 (1 H, b), 1.36 (1 H, b) |
| 34 | 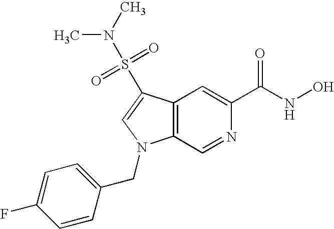 | 3-[(dimethylamino)sulfonyl]- 1-(4-fluorobenzyl-N- hydroxy-1H-pyrrolo[2,3- c]pyridine-5-carboxamide | (400 MHz, DMSO-D6) d ppm 2.61 (s, 6 H) 5.68 (s, 2 H) 7.20 (t, J = 8.81 Hz, 2 H) 7.46 (dd, J = 8.81, 5.54 Hz, 2 H) 8.37 (s, 1 H) 8.59 (s, 1 H) 8.98 (s, 1 H) 9.02 (s, 1 H) 11.29 (s, 1 H) |
| 35 | 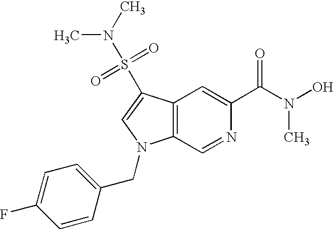 | 3-[(dimethylamino)sulfonyl]- 1-(4-fluorobenzyl)-N- hydroxy-N-methyl-1H- pyrrolo[2,3-c]pyridine-5- carboxamide | (300 MHz, MeOH) d ppm 2.72 (s, 6 H) 3.45 (s, 3 H) 5.71 (s, 2 H) 7.13 (t, J = 8.67 Hz, 2 H) 7.40 (dd, J = 8.57, 5.18 Hz, 2 H) 8.56 (s, 1 H) 8.74 (s, 1 H) 9.07 (s, 1 H) |
| 36 | 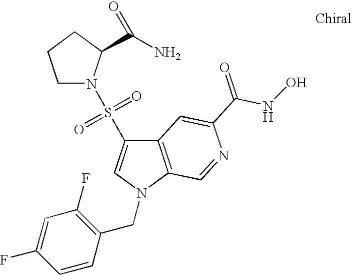 | 3-{[(2S)-2- (aminocarbonyl)pyrrolidin- 1-yl]sulfonyl}-1-(2,4- difluorobenzyl)-N-hydroxy- 1H-pyrrolo[2,3-c]pyridine- 5-carboxamide | (CD₃OD, 400 MHz) d 8.83 (1 H, s), 8.54 (1 H, s), 8.28 (1 H, s), 7.34 (1 H, m), 6.94 (2 H, m), 5.63 (2 H, s), 4.01 (2 H, dd), 3.54 (1 H, m), 1.87 (1 H, b), 1.75 (1 H, b), 1.68 (1 H, b), 1.48 (1 H, b) |
| 37 | 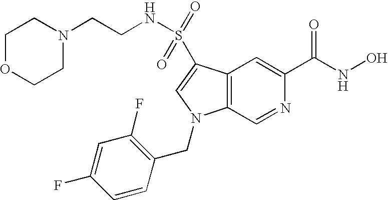 | 1-(2,4-difluorobenzyl)-N- hydroxy-3-{[(2-morpholin- 4-ylethyl)amino]sulfonyl}- 1H-pyrrolo[2,3-c]pyridine- 5-carboxamide | (CD₃OD, 400 MHz) d 8.83 (1 H, s), 8.51 (1 H, s), 8.12 (1 H, s), 7.38 (1 H, m), 6.94 (2 H, m), 5.59 (2 H, s), 3.37 (4 H, t), 2.95 (2 H, t), 2.30 (2 H, t), 2.18 (4 H, t), |
| 38 | 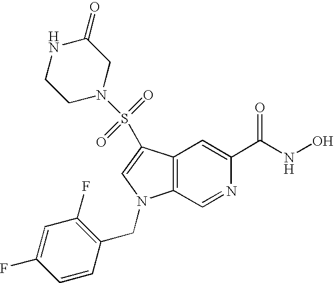 | 1-(2,4-difluorobenzyl)-N- hydroxy-3-[(3- oxopiperazin-1-yl)sulfonyl]- 1H-pyrrolo[2,3-c]pyridine- 5-carboxamide | (CD₃OD, 400 MHz) d 8.95 (1 H, s), 8.54 (1 H, s), 8.37 (1 H, s), 7.47 (1 H, m), 7.30 (1 H, m), 7.11 (1 H, m), 5.74 (2 H, s), 3.40 (2 H, s), 3.17 (2 H, t), 3.00 (2 H, t) |
| 39 | 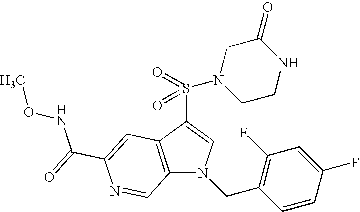 | 1-(2,4-difluorobenzyl)-N- methoxy-3-[(3- oxopiperazin-1-yl)sulfonyl]- 1H-pyrrolo[2,3-c]pyridine- 5-carboxamide | (CD₃OD, 400 MHz) d 8.98 (1 H, s), 8.63 (1 H, s), 8.42 (1 H, s), 7.47 (1 H, m), 7.09 (2 H, m), 5.76 (2 H, s), 3.87 (3 H, s), 3.72 (2 H, S), 3.40 (4 H, s) |
| 40 | 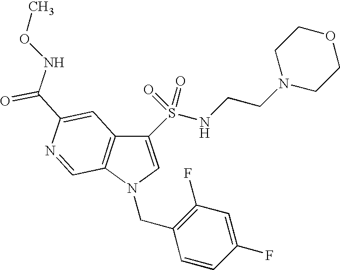 | 1-(2,4-difluorobenzyl)-N- methoxy-3-{[(2-morpholin- 4-ylethyl)amino]sulfonyl}- 1H-pyrrolo[2,3-c]pyridine- 5-carboxamide | (CD₃OD, 400 MHz) d 8.91 (1 H, s), 8.60 (1 H, s), 8.20 (1 H, s), 7.47 (1 H, m), 7.02 (2 H, m), 5.66 (2 H, s), 3.82 (3 H, s), 3.47 (4 H, t), 3.02 (2 H, t), 2.38 (2 H, t), 2.27 (4 H, t) |
| 41 | 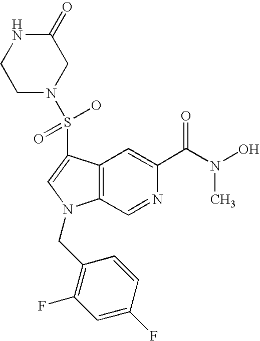 | 1-(2,4-difluorobenzyl)-N- hydroxy-N-methyl-3-[(3- oxopiperazin-1-yl)sulfonyl]- 1H-pyrrolo[2,3-c]pyridine- 5-carboxamide | (CD₃OD, 400 MHz) d 8.94 (1 H, s), 8.39 (1 H, s), 8.31 (1 H, s), 7.45 (1 H, m), 7.01 (2 H, m), 5.71 (2 H, s), 3.68 (2 H, s), 3.43 (3 H, s), 3.34 (4 H, s) |
| 42 | 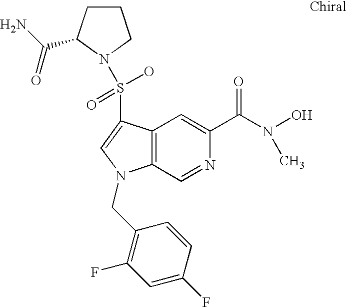 | 3-{[(2S)-2- (aminocarbonyl)pyrrolidin- 1-yl]sulfonyl)-1-(2,4- difluorobenzyl)-N-hydroxy- N-methyl-1H-pyrrolo[2,3- c]pyridine-5-carboxamide | (CD₃OD, 400 MHz) d 8.94 (1 H, s), 8.39 (1 H, s), 8.31 (1 H, s), 7.47 (1 H, m), 7.04 (2 H, m), 5.71 (2 H, s), 4.09 (1 H, m), 3.63 (2 H, m), 3.43 (3 H, s), 1.93 (1 H, b), 1.76 (2 H, b), 1.57 (1 H, b) |
| 43 | 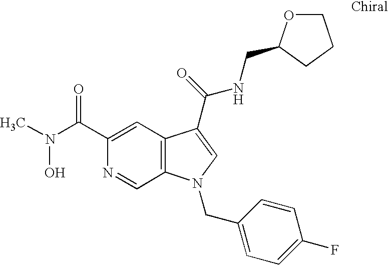 | 1-(4-fluorobenzyl)-N5- hydroxy-N5-methyl-N3- [(2S)-tetrahydrofuran-2- ylmethyl]-1H-pyrrolo[2,3- c]pyridine-3,5- dicarboxamide | (300 MHz, DMSO-D6) d ppm 1.53-1.65 (m, 1 H), 1.75-1.97 (m, 3 H), 3.34 (m, 5 H), 3.58-3.66 (m, 1 H), 3.75-3.83 (m, 1 H), 3.93-4.00 (m, 1 H), 5.60 (s, 2 H), 7.18-7.25 (m, 2 H), 7.37-7.46 (m, 2 H), 8.21- 8.29 (m, 1 H), 8.39 (s, 1 H), 8.41 (s, 1 H), 8.93 (s, 1 H) |
| 44 | 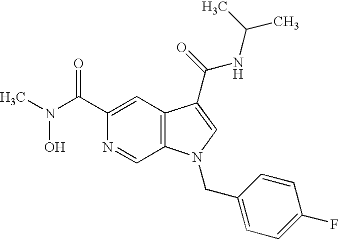 | 1-(4-fluorobenzyl)-N5- hydroxy-N3-isopropyl-N5- methyl-1H-pyrrolo[2,3- c]pyridine-3,5- dicarboxamide | (300 MHz, DMSO-D6) d ppm 1.17 (d, J = 6.56, 6 H), 3.32 (s, 3 H), 4.09 (heptet, J = 6.56, 1 H), 5.60 (s, 2 H), 7.17-7.26 (m, 2 H), 7.35-7.45 (m, 2 H), 7.95 (s, 0.5 H), 7.97 (s, 0.5 H), 8.41 (s, 1 H), 8.44 (s, 1 H), 8.96 (s, 1 H) |
| 45 | 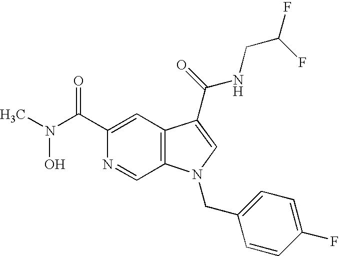 | N3-(2,2-difluoroethyl)-1-(4- fluorobenzyl)-N5-hydroxy- N5-methyl-1H-pyrrolo[2,3- c]pyridine-3,5- dicarboxamide | (DMSO, 300 MHz) d 8.93 (1 H, s), 8.39 (1 H, s), 8.37 (1 H, s), 7.39 (2 H, m), 7.18 (2 H, m), 6.10 (1 H, tt, J = 55.5, 3.9 Hz), 5.59 (3 H, s), 3.67 (2 H, m), 3.31 (3 H, s) |
| 46 | 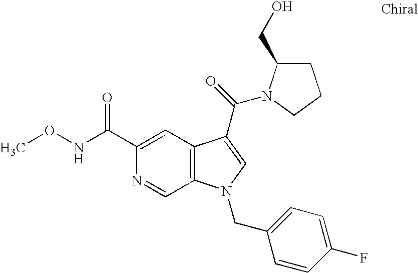 | 1-(4-fluorobenzyl)-3-{[2R)- 2-(hydroxymethyl)pyrrolidin- 1-yl]carbonyl}-N-methoxy- 1H-pyrrolo[2,3-c]pyridine- 5-carboxamide | (DMSO, 300 MHz) d 11.76 (1 H, s), 8.87 (1 H, s), 8.66 (1 H, s), 8.42 (1 H, s), 7.41 (2 H, m), 7.17 (2 H, m), 5.64 (2 H, s), 4.84 (1 H, m), 4.26 (1 H, m), 3.69 (3 H, s), 3.75-3.20 (3 H, m), 2.25-1.50 (4 H, m) |
| 47 | 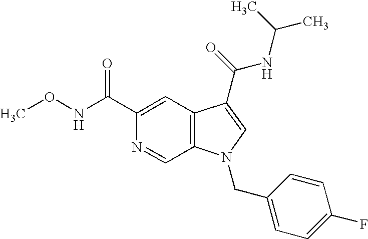 | 1-(4-fluorobenzyl)-N3- isopropyl-N5-methoxy-1H- pyrrolo[2,3-c]pyridine-3,5- dicarboxamide | (300 MHz, DMSO-D6) d ppm 1.17 (d, J = 6.56, 6 H), 3.69 (s, 3 H), 4.12 (heptet, J = 6.56, 1 H), 5.63 (s, 2 H), 7.19-7.28 (m, 2 H), 7.35-7.45 (m, 2 H), 7.96 (s, 0.5 H), 7.98 (s, 0.5 H), 8.39 (s, 1 H), 8.74 (s, 1 H), 8.92 (s, 1 H), 11.80 (s, 1 H) |
| 48 | 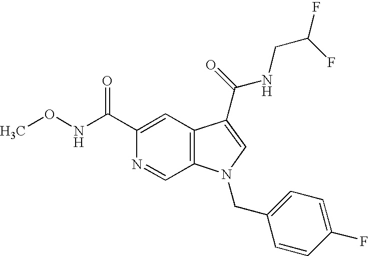 | N3-(2,2-difluoroethyl)-1-(4- fluorobenzyl)-N5-methoxy- 1H-pyrrolo[2,3-c]pyridine- 3,5-dicarboxamide | (DMSO, 300 MHz) d 8.92 (1 H, s), 8.71 (1 H, s), 8.53 (1 H, t, J = 6.0 (Hz), 8.40 (1 H, s), 7.38 (2 H, m), 7.21 (2 H, m), 6.11 (1 H, tt, J = 55.5, 3.9 Hz), 5.63 (2 H, s), 3.68 (3 H, s), 3.67 (2 H, m) |
| 49 | 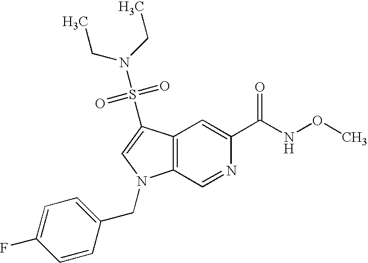 | 3-[(diethylamino)sulfonyl]- 1-(4-fluorobenzyl)-N- methoxy-1H-pyrrolo[2,3- c]pyridine-5-carboxamide | (CDCl3, 300 MHZ) d 11.8 (1 H, s), 8.96 (1 H, s), 8.41 (1 H, s), 7.42 (2 H, m), 7.16 (2 H, m), 5.66 (2 H, s), 3.68 (3 H, s), 3.17 (4 H, q, J = 6.9 Hz), 1.04 (6 H, t, J = 6.9 Hz) |
| 50 | 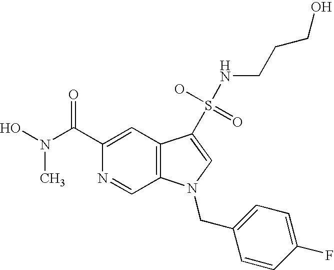 | 1-(4-fluorobenzyl)-N- hydroxy-3-{[(3- hydroxypropyl)amino] sulfonyl}-N-methyl-1H- pyrrolo[2,3-c]pyridine-5- carboxamide | (DMSO, 300 MHz) d 8.94 (1 H, s), 8.41 (1 H, s), 8.10 (1 H, s), 7.42 (2 H, m), 7.17 (2 H, m), 5.62 (2 H, s), 3.50 (3 H, s), 3.30 (5 H, m), 2.79 (1 H, t, J = 7.2 Hz), 1.49 (1 H, m) |
| 51 | 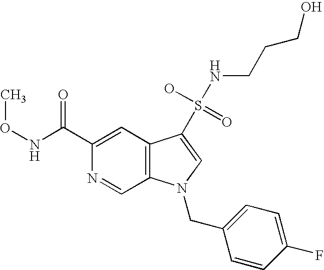 | 1-(4-fluorobenzyl)-3-{[(3- hydroxypropyl)amino]sulfonyl}- N-methoxy-1H- pyrrolo[2,3-c]pyridine-5- carboxamide | (DMSO, 300 MHZ) d 8.93 (1 H, s), 8.47 (1 H, s), 8.42 (1 H, s), 7.61 (1 H, bs), 7.40 (2 H, m), 7.16 (2 H, m), 5.65 (2 H, s), 3.68 (3 H, s), 3.30 (3 H, m), 2.79 (1 H, t, J = 7.2 Hz), 1.47 (1 H, m) |
| 52 | 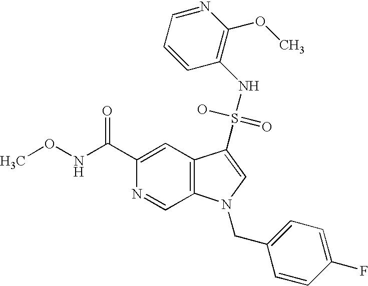 | 1-(4-fluorobenzyl)-N- methoxy-3-{[(2- methoxypyridin-3- yl)amino]sulfonyl}-1H- pyrrolo[2,3-c]pyridine-5- carboxamide | (CDCl3, 300 MHz) d 11.79 (1 H, s), 8.91 (1 H, s), 8.43 (1 H, s), 8.33 (1 H, s), 7.89 (1 H, d), 7.61 (1 H, d), 7.35 (2 H, m), 7.14 (2 H, m), 6.90 (1 H, dd), 5.58 (2 H, s), 3.69 (3 H, s), 3.14 (3 H, s) |
| 53 | 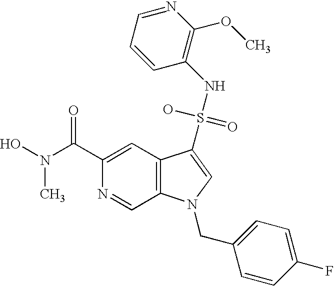 | 1-(4-fluorobenzyl)-N- hydroxy-3-{((2- methoxypyridin-3- yl)amino]sulfonyl}-N- methyl-1H- pyrrolo[2,3-c]pyridine- 5-carboxamide | (CDCl3, 300 MHz) d 9.76 (1 H, bs), 8.92 (1 H, s), 8.31 (1 H, s), 7.88 (1 H, d), 7.60 (1 H, d), 7.30 (2 H, m), 7.13 (2 H, m), 6.90 (1 H, dd), 5.54 (2 H, s), 3.40 (3 H, s), 3.19 (3 H, s) |
| 54 | 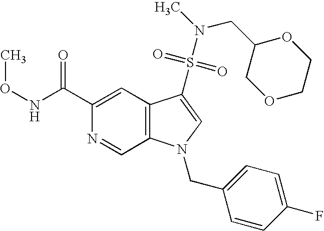 | 3-{[(1,4-dioxan-2- ylmethyl)(methyl)amino] sulfonyl}-1-(4- fluorobenzyl)-N- methoxy-1H- pyrrolo[2,3-c]pyridine- 5-carboxamide | (CDCl3, 300 MHz) d 10.18 (1 H, s), 8.69 (1 H, s), 8.58 (1 H, s), 7.76 (1 H, s), 7.40 (2 H, m), 7.16 (2 H, m), 5.41 (2 H, s), 3.90 (3 H, s), 3.89 - 3.50 (6 H, m), 3.40 (1 H, m), 3.11 (2 H, m), 2.90 (3 H, s) |
| 55 | 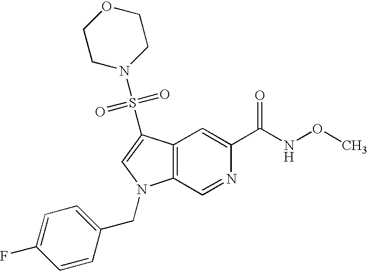 | 1-(4-fluorobenzyl)-N- methoxy-3- (morpholin-4- ylsulfonyl)-1H- pyrrolo[2,3-c]pyridine- 5-carboxamide | (400 MHz, CDCl3) d ppm 3.05-3.08 (m, 4 H), 3.75-3.92 (m, 4 H), 3.92 (s, 3 H), 5.46 (s, 2 H), 7.08-7.13 (m, 2 H), 7.18-7.20 (m, 2 H), 7.77 (s, 1 H), 8.63 (s, 1 H), 8.71 (s, 1 H), 10.24 (s, 1 H) |
| 56 | 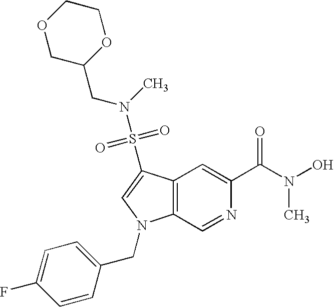 | 3-{[(1,4-dioxan-2- ylmethyl)(methyl)amino] sulfonyl}-1-(4- fluorobenzyl)-N- hydroxy-N-methyl- 1H-pyrrolo[2,3- c]pyridine-5- carboxamide | (400 MHz, CDCl3) d ppm 2.91 (s, 3 H), 3.04-3.21 (dd, J = 14.16, 6.08, 2 H), 3.37-3.45 (m, 1 H), 3.49 (brs, 1 H), 3.54-3.82 (m, 6 H), 3.83-3.91 (m, 1 H), 5.45 (s, 1 H), 7.07-7.15 (m, 2 H), 7.17- 7.24 (m, 2 H), 7.86 (brs, 1 H), 8.61 (brs, 1 H), 8.83 (brs, 1 H) |
| 57 | 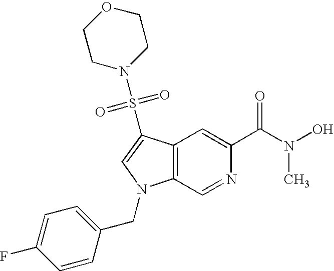 | 1-(4-fluorobenzyl)-N- hydroxy-N-methyl-3- (morpholin-4-ylsulfonyl)- 1H-pyrrolo[2,3-c]pyridine- 5-carboxamide | (400 MHz, MeOD) d ppm 2.95- 3.05 (m, 4 H), 3.43 (s, 3 H), 3.65- 3.80 (m, 3 H), 5.65 (s, 2 H), 7.05- 7.21 (m, 2 H), 7.29-7.48 (m, 2 H), 8.31 (brs, 1 H), 8.33 (s, 1 H), 8.88 (s, 1 H) |

### Example 43

### Integrase Strand-Transfer Scintillation Proximity Assay

Oligonucleotides: Oligonucleotide #1-5′-(biotin)CCCCTTTTAGTCAGTGTGGAAAATCTCTAGCA-3′ (SEQ ID NO: 1) and oligonucleotide #2-5′-ACTGCTAGAGATTTTCCACACTGACTAAAAG-3′ (SEQ ID NO: 2), were synthesized by TriLink BioTechnologies, Inc. (San Diego, Calif.). The annealed product represents preprocessed viral ds-DNA derived from the LTR U5 sequence of the viral genome. A ds-DNA control to test for non-specific interactions was made using a 3′ di-deoxy derivative of oligonucleotide #1 annealed to oligonucleotide #2. The CA overhang at the 5′ end of the non-biotinylated strand of the ds-DNA was created artificially by using a complimentary DNA oligonucleotide shortened by 2 base pairs. This configuration eliminates the requisite 3′ processing step of the integrase enzyme prior to the strand-transfer mechanism.

Host ds-DNA was prepared as an unlabeled and [³H]-thymidine labeled product from annealed oligonucleotide #3-5-AAAAAATGACCAAGGGCTAATTCACT-3′ (SEQ ID NO: 3), and oligonucleotide #4-1-5′-AAAAAAGTGAATTAGCCCTTGGTCA-3′ (SEQ ID NO: 4), both synthesized by TriLink BioTechnologies, Inc. (San Diego, Calif.). The annealed product had overhanging 3′ ends of poly(dA). Host DNA was custom radiolabeled by PerkinElmer Life Sciences Inc. (Boston, Mass.) using an enzymatic method with a 12/1 ratio of [methyl-³H]dTTP/cold ds-DNA to yield 5′-blunt end ds-DNA with a specific activity of >900 Ci/mmol. The radiolabeled product was purified using a NENSORB cartridge and stored in stabilized aqueous solution (PerkinElmer). The final radiolabeled product had six [³H]-thymidine nucleotides at both 5′ ends of the host ds-DNA.

Reagents: Streptavidin-coated polyvinyltoluene (PVT) SPA beads were purchased from Amersham Biosciences (Piscataway, N.J.). Cesium chloride was purchased from Shelton Scientific, Inc. (Shelton, Conn.). White, polystyrene, flat-bottom, non-binding surface, 96-well plates were purchased from Corning. All other buffer components were purchased from Sigma (St. Louis, Mo.) unless otherwise indicated.

Enzyme Construction Full-length wild type HIV-1 integrase (SF1) sequence (amino acids 1-289) was constructed in a pET24a vector (Novagen, Madison, Wis.). The construct was confirmed through DNA sequencing.

Enzyme Purification Full length wild-type HIV Integrase was expressed in E. coli BL21 (DE3) cells and induced with 1 mM isopropyl-1 thio-β-D-galactopyranoside (IPTG) when cells reached an optical density between 0.8-1.0 at 600 nm. Cells were lysed by microfluidation in 50 mM HEPES pH 7.0, 75 mM NaCl, 5 mM DTT, 1 mM 4-(2-Aminoethyl)benzenesulfonylfluoride HCl (AEBSF). Lysate was then centrifuged 20 minutes at 11 k rpm in GSA rotor in Sorvall RC-5B at 4° C. Supernant was discarded and pellet resuspended in 50 mM HEPES pH 7.0, 750 mM NaCl, 5 mM DTT, 1 mM AEBSF and homogenized in a 40 mL Dounce homogenizer for 20 minutes on ice. Homogenate was then centrifuged 20 minutes at 11 k rpm in SS34 rotor in Sorvall RC-5B at 4° C. Supernant was discarded and pellet resuspended in 50 mM HEPES pH 7.0, 750 mM NaCl, 25 mM CHAPS, 5 mM DTT, 1 mM AEBSF. Preparation was then centrifuged 20 minutes at 11 k rpm in SS34 rotor in Sorvall RC-5B at 4° C.

Supernant was then diluted 1:1 with 50 mM HEPES pH 7.0, 25 mM CHAPS, 1 mM DTT, 1 mM AEBSF and loaded onto a Q-Sepharose column pre-equilibrated with 50 mM HEPES, pH 7.0, 375 mM NaCl, 25 mM CHAPS, 1 mM DTT, 1 mM AEBSF. The flow through peak was collected and NaCl diluted to 0.1 M with 50 mM HEPES pH 7.0, 25 mM CHAPS, 1 mM DTT, 0.5 mM AEBSF and loaded onto a SP-Sepharose column pre-equilibrated with 50 mM HEPES pH 7.0, 100 mM NaCl, 25 mM CHAPS, 1 mM DTT, 0.5 mM AEBSF. After washing the column with the equilibration buffer, a 100 to 400 mM NaCl gradient was run. The eluted integrase was concentrated and run on a S-300 gel diffusion column using 50 mM HEPES pH 7.0, 500 mM NaCl, 25 mM CHAPS, 1 mM DTT, 0.5 mM AEBSF. The peak from this column was concentrated to 0.76 mg/mL and stored at −70° C. and later used for strand transfer assays. All columns were run in a 4° C. cold room.

Viral DNA Bead Preparation: Streptavidin-coated SPA beads were suspended to 20 mg/mL in 25 mM 3-morpholinopropanesulfonic acid (MOPS) (pH 7.2) and 1.0% NaN₃. Biotinylated viral DNA was bound to the hydrated SPA beads in a batch process by combining 25 pmoles of ds-DNA to 1 mg of suspended SPA beads (10 μL of 50 μM viral DNA to 1 mL of 20 mg/mL SPA beads). The mixture was incubated at 22° C. for a minimum of 20 min. with occasional mixing followed by centrifugation at 2500 rpm for 10 min. However, the centrifugation speed and time may vary depending upon the particular centrifuge and conditions. The supernatant was removed and the beads suspended to 20 mg/mL in 25 mM MOPS (pH 7.2) and 1.0% NaN₃. The viral DNA beads were stable for several weeks when stored at 4° C. Di-deoxy viral DNA was prepared in an identical manner to yield control di-deoxy viral DNA beads.

Preparation of Integrase-DNA Complex: Assay buffer was made as a 10× stock of 250 mM MOPS (pH 7.2), 500 mM NaCl, 50 mM 3-[(3-cholamidopropyl)dimethylammonio]-1-propanesulfonate (CHAPS), 0.5% (octylphenoxy)polyethoxyethanol (N P40) (IGEPAL-CA) and 0.05% NaN₃. Viral DNA beads were diluted to 2.67 mg/mL in 1× assay buffer plus 3 mM MgCl₂, 1% DMSO, and 10 mM fresh DTT. Integrase (IN) was pre-complexed to viral DNA beads in a batch process (IN/viral DNA/bead complex) by combining diluted viral DNA beads with integrase at a concentration of 385 nM followed by a minimum incubation time of 20 min. at 22° C. with gentle agitation. The sample was kept at 22° C. until transferred to the assay wells.

Preparation of Host DNA: Host DNA was prepared to 200 nM as a mixture of unlabeled and [³H]T-labeled host DNA diluted in 1× assay buffer plus 8.5 mM MgCl₂ and 15 mM DTT. Concentrations used were 4 nM [³H]T-labeled host DNA and 196 nM unlabeled host DNA. This ratio generates a SPA signal of 2000-3000 CPM in the absence of modulators such as inhibitors.

Strand-transfer Scintillation Proximity Assay: The strand-transfer reaction was carried out in 96-well microtiter plates, with a final enzymatic reaction volume of 100 μL. Ten microliters of compounds or test reagents diluted in 10% DMSO were added to the assay wells followed by the addition of 65 μL of the IN/viral-DNA/bead complex and mixed on a plate shaker. Then 25 μL of host DNA was added to the assay wells and mixed on a plate shaker. The strand-transfer reaction was initiated by transferring the assay plates to 37° C. dry block heaters. An incubation time of 50 min., which was shown to be within the linear range of the enzymatic reaction, was used. The final concentrations of integrase and host DNA in the assay wells were 246 nM and 50 nM, respectively.

The integrase strand-transfer reaction was terminated by adding 70 μL of stop buffer (150 mM EDTA, 90 mM NaOH, and 6 M CsCl) to the wells. Components of the stop buffer function to terminate enzymatic activity (EDTA), dissociate integrase/DNA complexes in addition to separating non-integrated DNA strands (NaOH), and float the SPA beads to the surface of the wells to be in closer range to the PMT detectors of the TopCount® plate-based scintillation counter (PerkinElmer Life Sciences Inc. (Boston, Mass.)). After the addition of stop buffer, the plates were mixed on a plate shaker, sealed with transparent tape, and allowed to incubate a minimum of 60 min. at 22° C. The assay signal was measured using a TopCount® plate-based scintillation counter with settings optimal for [³H]-PVT SPA beads. The TopCount® program incorporated a quench standardization curve to normalize data for color absorption of the compounds. Data values for quench-corrected counts per minute (QCPM) were used to quantify integrase activity. Counting time was 2 min./well.

The di-deoxy viral DNA beads were used to optimize the integrase strand-transfer reaction. The di-deoxy termination of the viral ds-DNA sequence prevented productive integration of viral DNA into the host DNA by integrase. Thus, the assay signal in the presence of di-deoxy viral DNA was a measure of non-specific interactions. Assay parameters were optimized to where reactions with di-deoxy viral DNA beads gave an assay signal closely matched to the true background of the assay. The true background of the assay was defined as a reaction with all assay components (viral DNA and [³H]-host DNA) in the absence of integrase.

Determination of Compound Activity: The percent inhibition of the compound was calculated using the equation (1−((QCPM sample−QCPM min)/(QCPM max−QCPM min)))*100. The min value is the assay signal in the presence of a known inhibitor at a concentration 100-fold higher than the IC₅₀ for that compound. The min signal approximates the true background for the assay. The max value is the assay signal obtained for the integrase-mediated activity in the absence of compound (i.e. with DMSO instead of compound in DMSO).

Compounds were prepared in 100% DMSO at 100-fold higher concentrations than desired for testing in assays (generally 5 mM), followed by dilution of the compounds in 100% DMSO to generate an 11-point titration curve with ½-log dilution intervals. The compound sample was further diluted 10-fold with water and transferred to the assay wells. The percentage inhibition for an inhibitory compound was determined as above with values applied to a nonlinear regression, sigmoidal dose response equation (variable slope) using GraphPad Prism curve fitting software (GraphPad Software, Inc., San Diego, Calif.). Concentration curves were assayed in duplicate and then repeated in an independent experiment.

### Example 44

### HIV-1 Cell Protection Assay

The antiviral activities of potential modulator compounds (test compounds) were determined in HIV-1 cell protection assays using the RF strain of HIV-1, CEM-SS cells, and the XTT dye reduction method (Weislow, O, S. et al., J. Natl. Cancer Inst 81: 577-586 (1989)). Subject cells were infected with HIV-1 RF virus at an moi of to affect about a 90% kill (for example, an moi in the range of from about 0.025 to about 0.819) or mock infected with medium only and added at 2×10⁴ cells per well, with the addition of approximately 200 μL of medium, into 96 well plates containing half-log dilutions of test compounds. Six days later, 50 μl of XTT solution (1 mg/ml XTT tetrazolium and 20 nM phenazine methosulfate) were added to the wells and the plates were reincubated for four hours. Viability, as determined by the amount of XTT formazan produced, was quantified spectrophotometrically by absorbance at 450 nm.

Data from CPE assays were expressed as the percent of formazan produced in compound-treated cells compared to formazan produced in wells of uninfected, compound-free cells. The fifty percent effective concentration (EC₅₀) was calculated as the concentration of compound that affected an increase in the percentage of formazan production in infected, compound-treated cells to 50% of that produced by uninfected, compound-free cells. The 50% cytotoxicity concentration (CC₅₀) was calculated as the concentration of compound that decreased the percentage of formazan produced in uninfected, compound-treated cells to 50% of that produced in uninfected, compound-free cells. The therapeutic index was calculated by dividing the cytotoxicity (CC₅₀) by the antiviral activity (EC₅₀).

### Example 45

### Antiviral Data

| Example No. | IC₅₀ (nM) | EC₅₀ (nM) |
|---|---|---|
| 1 | 130 | 11 |
| 2 | | 20 |
| 3 | 40.5 | 21 |
| 4 | 360 | 42 |
| 5 | 56 | 5.2 |
| 6 | 270 | 30 |
| 7 | 550 | 62 |
| 8 | 1400 | 700 |
| 9 | 267 | 42 |
| 10 | 1145 | 40 |
| 11 | 530 | 67 |
| 12 | 801 | 140 |
| 13 | 374 | 66 |
| 14 | 2600 | 5400 |
| 15 | 300 | 120 |
| 16 | 211 | 180 |
| 17 | 168 | 110 |
| 18 | 230 | 460 |
| 19 | 105 | 18 |
| 20 | 370 | 580 |
| 21 | 102 | 71 |
| 22 | 320 | 170 |
| 23 | 75 | 57 |
| 24 | 16 | 380 |
| 25 | 152 | 250 |
| 26 | 158 | 90 |
| 27 | 76 | 38 |
| 28 | 46 | 100 |
| 29 | 70 | 130 |
| 30 | 72 | 110 |
| 31 | 141 | 200 |
| 32 | 503 | 210 |
| 33 | 320 | 140 |
| 34 | 136 | 47 |
| 35 | 1030 | 370 |
| 36 | 197 | 340 |
| 37 | 161 | 490 |
| 38 | 265 | 8000 |
| 39 | 299 | 210 |
| 40 | 760 | 52 |
| 41 | 1148 | 3300 |
| 42 | 1050 | 6300 |
| 43 | 221 | 80 |
| 44 | 205 | 70 |
| 45 | 243 | 39 |
| 46 | 895 | 56 |
| 47 | 249 | 32 |
| 48 | 510 | 29 |
| 49 | | 17 |
| 50 | | 460 |
| 51 | | 110 |
| 52 | | 52 |
| 53 | | 140 |
| 54 | | 48 |
| 55 | | 25 |
| 56 | | 400 |
| 57 | | 480 |

1. A compound of formula (I), 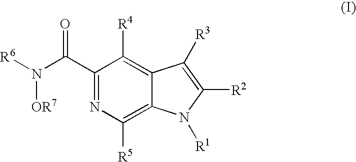
wherein:
R¹ is hydrogen, C₁-C₈ alkyl, C₂-C₈ alkenyl, or C₁-C₈ heteroalkyl, wherein said C₁-C₈ alkyl, C₂-C₈ alkenyl, or C₁-C₈ heteroalkyl groups may be optionally substituted with at least one substituent independently selected from halo, —OR^{12a}, —N(R^{12a}R^{12b}), —C(O)N(R^{12a}R^{12b}), —NR^{12a}C(O)N(R^{12a}R^{12b}), —NR^{12a}C(O)R^{12a}, —NR^{12a}C(NR^{12a})N(R^{12a}R^{12b}), —SR^{12a}, S(O)R^{12a}, S(O)₂R^{12a}, —S(O)₂N(R^{12a}R^{12b})₂, C₁-C₈ alkyl, C₆-C₁₄ aryl, C₃-C₈ cycloalkyl, and C₂-C₉ heteroaryl, wherein said C₁-C₈ alkyl, C₆-C₁₄ aryl, C₃-C₈ cycloalkyl, and C₂-C₉ heteroaryl groups are optionally substituted with at least one substituent independently selected from halo, —C(R^{12a}R^{12b}R^{12c}), —OH, and C₁-C₈ alkoxy;
R² is hydrogen;
R³ is —NR⁸C(O)R⁹, —NR⁸S(O)R⁹, —NR⁸S(O)₂R⁹, —C(O)NR⁸R⁹, —S(O)NR⁸R⁹, or —S(O)₂NR⁸R⁹;
R⁴ is hydrogen, halo, C₁-C₈ alkyl, —OR^{12a}, —NR^{12a}R^{12b}, C₁-C₈ heteroalkyl C₂-C₈ alkenyl, or C₂-C₈ alkynyl, wherein said C₁-C₈ alkyl, C₁-C₈ heteroalkyl, C₂-C₈ alkenyl or C₂-C₈ alkynyl groups are optionally substituted with at least one R¹³;
R⁵ is hydrogen;
R⁶ is hydrogen, C₁-C₈ alkyl, C₁-C₈ heteroalkyl, or C₂-C₈ alkenyl, wherein said C₂-C₈ alkenyl is optionally substituted with at least one —OR^{12a} group;
R⁷ is hydrogen, C₁-C₈ heteroalkyl, C₆-C₁₄ aryl, C₂-C₈ alkenyl, or C₁-C₈ alkyl, wherein said C₁-C₈ alkyl is optionally substituted with at least one C₃-C₈ cycloalkyl or C₆-C₁₄ aryl group;
each R⁸ and R⁹, which may be the same or different, is independently selected from hydrogen, C₁-C₈ alkyl, C₃-C₈ cycloalkyl, C₆-C₁₂ aryl, C₂-C₈ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group; or
R⁸ and R⁹, together with the nitrogen atom to which they are attached, form a C₂-C₉ heterocyclyl or a C₂-C₉ heteroaryl group, each of which is optionally substituted with at least one R¹⁰ group;
each R¹⁰ is independently selected from halo, C₁-C₈ alkyl, C₃-C₈ cycloalkyl, C₆-C₁₂ aryl, C₂-C₉ heterocyclyl, C₂-C₉ heteroaryl, —(CR^{12a}R^{12b})ₜOR⁷, —C(O)R^{12a}, —S(O)₂R⁷, (CR^{12a}R^{12b})_{z}C(O)NR^{12a}R^{12b}, —NR^{12a}R^{12b}, and —CF₃, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted with at least one R¹⁴ group;
each R^{12a}, R^{12b}, and R^{12c}, which may be the same or different, is independently selected from hydrogen and C₁-C₈ alkyl;
each R¹³, which may be the same or different, is independently selected from —OR^{12a}, halo, C₆-C₁₄ aryl, C₂-C₉ heteroaryl, C₁-C₈ heteroalkyl, C₃-C₈ cycloalkyl, C₂-C₉ heterocyclyl, and —C(R^{12a}R^{12b}R^{12c});
each R¹⁴, which may be the same or different, is independently selected from halogen, C₁-C₈ alkyl, C₃-C₈ cycloalkyl, —CF₃, and —OR^{12a};
each z, which may be the same or different, is independently selected and is 0, 1, or 2; and
each t, which may be the same or different, is independently selected and is 0, 1, 2, or 3; or
a pharmaceutically acceptable salt or solvate thereof.

2. A compound according to claim 1, wherein R¹ is —(CH₂)(C₆-C₁₄ aryl), wherein said C₆-C₁₄ aryl is optionally substituted with at least one halo, or a pharmaceutically acceptable salt or solvate thereof.

3. A compound according to claim 1, wherein each R⁸ and R⁹, which may be the same or different, is independently selected from hydrogen, C₁-C₈ alkyl, C₃-C₈ cycloalkyl, C₆-C₁₂ aryl, C₂-C₉ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group, or a pharmaceutically acceptable salt or solvate thereof.

4. A compound according to claim 1, wherein R⁸ and R⁹, together with the nitrogen atom to which they are attached, form a C₂-C₉ heterocyclyl group, which is optionally substituted with at least one R¹⁰ group, or a pharmaceutically acceptable salt or solvate thereof.

5. A compound according to claim 1, wherein:
R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy;
R³ is —NR⁸C(O)R⁹, —NR⁸S(O)R⁹, —NR⁸(O)₂R⁹, —C(O)NR⁸R⁹, —S(O)NR⁸R⁹, or —S(O)₂NR⁸R⁹;
R⁴ is hydrogen;
R⁶ is hydrogen or C₁-C₈ alkyl;
R⁷ is hydrogen or C₁-C₈ alkyl; and
R⁸ and R⁹, which may be the same or different, is independently selected from hydrogen, C₁-C₈ alkyl, C₃-C₈ cycloalkyl, C₁-C₁₂ aryl, C₂-C₉ heterocyclyl, and C₂-C₉ heteroaryl, wherein each of said C₁-C₈ alkyl, C₃-C₈ cycloalkyl, C₆-C₁₂ aryl, C₂-C₉ heterocyclyl, and C₂-C₉ heteroaryl groups may be optionally substituted by at least one R¹⁰ group; or
a pharmaceutically acceptable salt or solvate thereof.

6. A compound according to claim 5, wherein R¹ is —(CH₂)(C₆-C₁₄ aryl), wherein said C₆-C₁₄ aryl is optionally substituted with at least one halo, or a pharmaceutically acceptable salt or solvate thereof.

7. A compound according to claim 1, wherein:
R¹ is C₁-C₈ alkyl, wherein said C₁-C₈ alkyl is substituted with C₆-C₁₄ aryl wherein said C₆-C₁₄ aryl is optionally substituted with at least one substituent independently selected from halo, —C(R^{12a}R^{12b}R^{12c}), —OH, and C₁-C₈ alkoxy;
R³ is —NR⁸C(O)R⁹, —NR⁸S(O)R⁹, —NR⁸S(O)₂R⁹, —C(O)NR⁸R⁹, —S(O)NR⁸R⁹, or —S(O)₂NR⁸R⁹;
R⁴ is hydrogen;
R⁶ is hydrogen or C₁-C₈ alkyl;
R⁷ is hydrogen or C₁-C₈ alkyl; and
R⁸ and R⁹, together with the nitrogen atom to which they are attached, form a C₂-C₉ heterocyclyl group, which is optionally substituted with at least one R¹⁰ group; or
a pharmaceutically acceptable salt or solvate thereof.

8. A compound according to claim 7, wherein R¹ is —(CH₂)(C₆-C₁₄ aryl), wherein said C₆-C₁₄ aryl is optionally substituted with at least one halo, or a pharmaceutically acceptable salt or solvate thereof.

9. A compound according to claim 1, selected from:
3-(acetylamino)-1-(4-fluorobenzyl)-N-hydroxy-1H-pyrrolo[2,3-c]pyridine-carboxamide;
3-(acetylamino)-1-(4-fluorobenzyl)-N-hydroxy-N-methyl-1H-pyrrolo[2,3-c]pyridine-5-carboxamide;
1-(4-fluorobenzyl)-N-hydroxy-3-{(phenylsulfonyl)amino}-1H-pyrrolo[2,3-c]pyridine-5-carboxamide;
1-(4-fluorobenzyl)-N-hydroxy-N-methyl-3-{(phenylsulfonyl)amino}-1H-pyrrolo[2,3-c]pyridine-5-carboxamide;
1-(4-fluorobenzyl)-N-hydroxy-3-[(methylsulfonyl)amino]-1H-pyrrolo[2,3-c]pyridine-5-carboxamide;
1-(4-fluorobenzyl)-N-hydroxy-N-methyl-3-[(methylsulfonyl)amino]-1H-pyrrolo[2,3-c]pyridine-5-carboxamide;
1-(4-fluorobenzyl)-N-5-methoxy-N-3-(2-morpholin-4-ylethyl)-1H-pyrrolo[2,3-c]pyridine-3,5-dicarboxamide;
N-3-[(1-ethylpyrrolidin-2-yl)methyl]-1-(4-fluorobenzyl)-N-5-methoxy-1H-pyrrolo[2,3-c]pyridine-3,5-dicarboxamide;
1-(4-fluorobenzyl)-N-5-methoxy-N-3-[3-(2-oxopyrrolidin-1-yl)propyl]-1H-pyrrolo[2,3-c]pyridine-3,5-dicarboxamide;
1-(4-fluorobenzyl)-3-{[(2S)-2-(hydroxymethyl)pyrrolidin-1-yl]carbonyl}-N-methoxy-1H-pyrrolo[2,3-c]pyridine-5-carboxamide;
N-3-[(1,5-dimethyl-1H-pyrazol-4-yl)methyl]-1-(4-fluorobenzyl)-N-5-methoxy-1H-pyrrolo[2,3-c]pyridine-3,5-dicarboxamide;
3-{[3-(dimethylamino)pyrrolidin-1-yl]carbonyl}-1-(4-fluorobenzyl)-N-methoxy-1H-pyrrolo[2,3-c]pyridine-5-carboxamide;
1-(4-fluorobenzyl)-3-{[3-(hydroxymethyl)piperidin-1-yl]carbonyl}-N-methoxy-1H-pyrrolo[2,3-c]pyridine-5-carboxamide;
1-(4-fluorobenzyl)-N-5-hydroxy-N-5-methyl-N-3-(tetrahydro-1H-pyrrolizin-7a(5H)-ylmethyl)-1H-pyrrolo[2,3-c]pyridine-3,5-dicarboxamide;
N-3-[1-cyclopropyl-3-(cyclopropylamino)-3-oxopropyl]-1-(4-fluorobenzyl)-N-5-methoxy-1H-pyrrolo[2,3-c]pyridine-3,5-dicarboxamide;
1-(4-fluorobenzyl)-N-5-hydroxy-N-5-methyl-N-3-[3-(2-oxopyrrolidin-1-yl)propyl]-1H-pyrrolo[2,3-c]pyridine-3,5-dicarboxamide;
N-3-[(1,5-dimethyl-1H-pyrazol-4-yl)methyl]-1-(4-fluorobenzyl)-N-5-hydroxy-N-5-methyl-1H-pyrrolo[2,3-c]pyridine-3,5-dicarboxamide;
N-3-[1-cyclopropyl-3-(cyclopropylamino)-3-oxopropyl]-1-(4-fluorobenzyl)-N-5-hydroxy-N-5-methyl-1H-pyrrolo[2,3-c]pyridine-3,5-dicarboxamide;
3-[(benzylsulfonyl)amino]-1-(4-fluorobenzyl)-N-hydroxy-1H-pyrrolo[2,3-c]pyridine-5-carboxamide;
1-(4-fluorobenzyl)-N-hydroxy-3-[(1,2,3,4-tetrahydroisoquinolin-7-ylsulfonyl)amino]-1H-pyrrolo[2,3-c]pyridine-5-carboxamide;
3-{[(5-chloro-2-thienyl)sulfonyl]amino}1-(4-fluorobenzyl)-N-hydroxy-1H-pyrrolo[2,3-c]pyridine-5-carboxamide;
1-(4-fluorobenzyl)-N-hydroxy-3-{[(2S)-2-(hydroxymethyl)pyrrolidin-1-yl]carbonyl}-N-methyl-1H-pyrrolo[2,3-c]pyridine-5-carboxamide;
1-(4-fluorobenzyl)-N-hydroxy-3-{[(2S)-2-(hydroxymethyl)pyrrolidin-1-yl]carbonyl}-1H-pyrrolo[2,3-c]pyridinecarboxamide;
N-3-[1-cyclopropyl-3-(cyclopropylamino)-3-oxopropyl]-4-fluorobenzyl)-N-5-hydroxy-1H-pyrrolo[2,3-c]pyridine-3,5-dicarboxamide;
1-(4-fluorobenzyl)-N-5-hydroxy-N-3-tetrahydro-1H-pyrrolizin-7a(5H)-ylmethyl)-1H-pyrrolo[2,3-c]pyridine-3,5-dicarboxamide;
3-{[3-(dimethylamino)pyrrolidin-1-yl]carbonyl}1-(4-fluorobenzyl)-N-hydroxy-1H-pyrrolo[2,3-c]pyridine-5-carboxamide;
1-(4-fluorobenzyl)-N-5-hydroxy-N-3-(2-morpholin-ylethyl)-1H-pyrrolo[2,3-c]pyridine-3,5-dicarboxamide;
N-3-[(1,5-dimethyl-1H-pyrazol-4-yl)methyl]-1-(4-fluorobenzyl)-N-5-hydroxy-1H-pyrrolo[2,3-c]pyridine-3,5-dicarboxamide;
1-(4-fluorobenzyl)-N-5-hydroxy-N-3-[3-(2-oxopyrrolidin-1-yl)propyl]-1H-pyrrolo[2,3-c]pyridine-3,5-dicarboxamide;
1-(4-fluorobenzyl)-N-hydroxy-3-{[3-(hydroxymethyl)piperidin-1-yl]carbonyl}-1H-pyrrolo[2,3-c]pyridine-5-carboxamide;
N-3-[(1-ethylpyrrolidin-2-yl)methyl]-1-(4-fluorobenzyl)-N-5-hydroxy-1H-pyrrolo[2,3-c]pyridine-3,5-dicarboxamide;
1-(4-fluorobenzyl)-N-5-hydroxy-N-5-methyl-N-3-(2-morpholin-4-ylethyl)-1H-pyrrolo[2,3-c]pyridine-3,5-dicarboxamide;
1-(4-fluorobenzyl)-N-hydroxy-3-{[3-(hydroxymethyl)piperidin-1-yl]carbonyl}-N-methyl-1H-pyrrolo[2,3-c]pyridine-5-carboxamide;
3-[(dimethylamino)sulfonyl]-1-(4-fluorobenzyl)-N-hydroxy-1H-pyrrolo[2,3-c]pyridine-5-carboxamide;
3-[(dimethylamino)sulfonyl]-1-(4-fluorobenzyl)-N-hydroxy-N-methyl-1H-pyrrolo[2,3-c]pyridine-5-carboxamide;
3-{[(2S)-2-(aminocarbonyl)pyrrolidin-1-yl]sulfonyl}-1-(2,4-difluorobenzyl)-N-hydroxy-1H-pyrrolo[2,3-c]pyridine-5-carboxamide;
1-(2,4-difluorobenzyl)-N-hydroxy-3-{[(2-morpholin-4-ylethyl)amino]sulfonyl}-1H-pyrrolo[2,3-c]pyridine-5-carboxamide;
1-(2,4-difluorobenzyl)-N-hydroxy-3-[(3-oxopiperazin-1-yl)sulfonyl]-1H-pyrrolo[2,3-c]pyridine-5-carboxamide;
1-(2,4-difluorobenzyl)-N-methoxy-3-[(3-oxopiperazin-1-yl)sulfonyl]-1H-pyrrolo[2,3-c]pyridine-5-carboxamide;
1-(2,4-difluorobenzyl)-N-methoxy-3-{[(2-morpholin-4-ylethyl)amino]sulfonyl}-1H-pyrrolo[2,3-c]pyridine-5-carboxamide;
1-(2,4-difluorobenzyl)-N-hydroxy-N-methyl-3-[(3-oxopiperazin-1-yl)sulfonyl]-1H-pyrrolo[2,3-c]pyridine-5-carboxamide;
3-{[(2S)-2-(aminocarbonyl)pyrrolidin-1-yl]sulfonyl}-1-(2,4-difluorobenzyl)-N-hydroxy-N-methyl-1H-pyrrolo[2,3-c]pyridine-5-carboxamide;
1-(4-fluorobenzyl)-N5-hydroxy-N5-methyl-N3-[(2S)-tetrahydrofuran-2-ylmethyl]-1H-pyrrolo[2,3-c]pyridine-3,5-dicarboxamide;
1-(4-fluorobenzyl)-N5-hydroxy-N3-isopropyl-N5-methyl-1H-pyrrolo[2,3-c]pyridine-3,5-dicarboxamide;
N3-(2,2-difluoroethyl)-1-(4-fluorobenzyl)-N5-hydroxy-N5-methyl-1H-pyrrolo[2,3-c]pyridine-3,5-dicarboxamide;
1-(4-fluorobenzyl)-3-{[(2R)-2-(hydroxymethyl)pyrrolidin-1-yl]carbonyl}N-methoxy-1H-pyrrolo[2,3-c]pyridine-5-carboxamide;
1-(4-fluorobenzyl)-N3-isopropyl-N5-methoxy-1H-pyrrolo[2,3-c]pyridine-3,5-dicarboxamide;
N3-(2,2-difluoroethyl)-1-(4-fluorobenzyl)-N5-methoxy-1H-pyrrolo[2,3-c]pyridine-3,5-dicarboxamide;
3-[(diethylamino)sulfonyl]-1-(4-fluorobenzyl)-N-methoxy-1H-pyrrolo[2,3-c]pyridine-5-carboxamide;
1-(4-fluorobenzyl)-N-hydroxy-3-{[(3-hydroxypropyl)amino]sulfonyl}-N-methyl-1H-pyrrolo[2,3-c]pyridine-5-carboxamide;
1-(4-fluorobenzyl)-3-{[(3-hydroxypropyl)amino]sulfonyl}-N-methoxy-1H-pyrrolo[2,3-c]pyridine-5-carboxamide;
1-(4-fluorobenzyl)-N-methoxy-3-{[(2-methoxypyridin-3-yl)amino]sulfonyl}-1H-pyrrolo[2,3-c]pyridine-5-carboxamide;
1-(4-fluorobenzyl)-N-hydroxy-3-{[(2-methoxypyridin-3-yl)amino]sulfonyl}-N-methyl-1H-pyrrolo[2,3-c]pyridine-5-carboxamide;
3-{[(1,4-dioxan-2-ylmethyl)(methyl)amino]sulfonyl}-1-(4-fluorobenzyl)-N-methoxy-1H-pyrrolo[2,3-c]pyridine-5-carboxamide;
1-(4-fluorobenzyl)-N-methoxy-3-(morpholin-4-ylsulfonyl)-1H-pyrrolo[2,3-c]pyridine-5-carboxamide;
3-{[(1,4-dioxan-2-ylmethyl)(methyl)amino]sulfonyl}-1-(4-fluorobenzyl)-N-hydroxy-N-methyl-1H-pyrrolo[2,3-c]pyridine-5-carboxamide; and
1-(4-fluorobenzyl)-N-hydroxy-N-methyl-3-(morpholin-4-ylsulfonyl)-1H-pyrrolo[2,3-c]pyridine-5-carboxamide; or a pharmaceutically acceptable salt or solvate thereof.

10. A pharmaceutical composition, comprising a therapeutically effective amount of a compound according to claim 1, or a pharmaceutically acceptable salt or solvate thereof, and a pharmaceutically acceptable carrier or diluent.

11. A method of inhibiting HIV replication in a mammal, comprising administering to said mammal an HIV-inhibiting amount of a compound according to claim 1, or a pharmaceutically acceptable salt or solvate thereof.

12. A method of inhibiting HIV replication in a cell, comprising contacting said cell with an HIV-inhibiting amount of a compound according to claim 1, or a pharmaceutically acceptable salt or solvate thereof.

13. A method of inhibiting HIV integrase enzyme activity, comprising contacting said integrase enzyme with a HIV integrase-inhibiting amount of a compound according to claim 1, or a pharmaceutically acceptable salt or solvate thereof.

14. (canceled)

15. A method of treating acquired immune deficiency syndrome in a mammal, comprising administering to said mammal a therapeutically effective amount of a compound according to claim 1, or a pharmaceutically acceptable salt or solvate thereof.
